(19) United States
(12) Patent Application Publication
(10) Pub. No.: US 2024/0109965 A1
(43) Pub. Date: Apr. 4, 2024

(54) IMMUNOGLOBULIN SINGLE VARIABLE DOMAINS TARGETING T CELL RECEPTOR

(71) Applicants: Ablynx N.V., Zwijnaarde (BE); Sanofi, Paris (FR)

(72) Inventors: Melissa Dullaers, Zwijnaarde (BE); Katrijn Neyt, Zwijnaarde (BE); Annelies Roobrouck, Zwijnaarde (BE); Stephanie Staelens, Zwijnaarde (BE); Soren Steffensen, Zwijnaarde (BE); Tom Van Belle, Zwijnaarde (BE); Diane Van Hoorick, Zwijnaarde (BE); Judith Verhelst, Zwijnaarde (BE)

(73) Assignees: Ablynx N.V., Zwijnaarde (BE); Sanofi, Paris (FR)

(21) Appl. No.: 18/334,642

(22) Filed: Jun. 14, 2023

(30) Foreign Application Priority Data
Jun. 14, 2022 (EP) 22305869.4

(51) Int. Cl. C07K 16/28; A61P 35/00

(52) U.S. Cl. CPC C07K 16/2809; A61P 35/00; C07K 2317/14; C07K 2317/565; C07K 2317/567; C07K 2317/569

(57) ABSTRACT

The present technology provides immunoglobulin single variable domains (ISVDs) binding both the constant domain of a human T cell receptor (TCR) on a T cell and the constant domain of a non-human primate TCR on a T cell. It also relates to polypeptides comprising an ISVD according to the present technology and at least one ISVD capable of binding to an antigen on a target cell. The present technology further provides nucleic acids encoding said ISVDs or polypeptides as well as vectors, hosts and methods to produce these ISVDs or polypeptides. Moreover, the present technology relates to methods for treatment making use of the ISVDs or polypeptides of the present technology.

### REFERENCE TO AN ELECTRONIC SEQUENCE LISTING

The contents of the electronic sequence listing (A084870226US00-SEQ-CRP.xml; Size: 297,367 bytes; and Date of Creation: Jun. 13, 2023) is herein incorporated by reference in its entirety.

### 1 FIELD OF THE PRESENT TECHNOLOGY

The present technology provides immunoglobulin single variable domains (ISVDs) binding both the constant domain of a human T cell receptor (TCR) on a T cell and the constant domain of a non-human primate TCR on a T cell. It also relates to multispecific polypeptides comprising an ISVD according to the present technology and at least one ISVD capable of binding to an antigen on a target cell. The present technology further provides nucleic acids encoding said ISVDs or polypeptides as well as vectors, hosts and methods to produce these ISVDs or polypeptides. Moreover, the present technology relates to methods for treatment making use of the ISVDs or polypeptides according to the present technology.

### 2 TECHNOLOGICAL BACKGROUND

Cancer takes an enormous human toll around the world. It is nowadays the second leading cause of death globally, only preceded by heart disease and stroke. Cancers figure among the leading causes of morbidity and mortality worldwide, with approximately 19.3 million new cases and 10 million cancer related deaths in 2020. The number of new cases is expected to rise further over the next decades. Population growth, ageing and lifestyle changes have been described as contributing factors to the increasing cancer burden. In 2013, the WHO projected that by 2030 cancer will surpass ischemic heart disease and become the most common cause of death worldwide (source: WHO Cancer).

The total economic impact of premature death and disability from cancer worldwide was already about $900 billion in 2008, which represented 1.5% of the world's gross domestic product at that time. With cancer becoming increasingly common, the total economic impact is sure to have increased significantly as well. Available treatment regimens for solid tumors typically include a combination of surgical resection, chemotherapy, and radiotherapy. In 40 years of clinical experience little progress has been achieved, especially in advanced stages of cancer. New therapies combatting cancer are eagerly awaited.

Antibody therapy is now an important part of the physician's armamentarium to battle diseases and especially cancer. Monoclonal antibodies have been established as a key therapeutic approach for a range of diseases already for several years. Most of the contemporaneously approved antibody therapies rely on monospecific monoclonal antibodies (mAbs). Until today, most of the targets of the mAbs require either an agonistic or an antagonistic approach. Whereas targeting of cell-surface antigens themselves can mediate antitumor activity through the induction of apoptosis, most mAb-based activity against hematologic malignancies is reliant on either Fc-mediated effector functions such as complement dependent cytotoxicity (CDC) and antibody-dependent cell-mediated cytotoxicity (ADCC).

More recently, immunotherapy has emerged as a rapidly growing area of cancer research. Immunotherapy is directing the body's immune surveillance system, and in particular T cells, to cancer cells.

Cytotoxic T cells (CTL) are T lymphocytes that kill cancer cells, cells that are infected (particularly with viruses), or cells that are damaged in other ways. T lymphocytes (also called T cells) express the T cell receptor (TCR) and the CD3 receptor on the cell surface. The αβTCR-CD3 complex (or “TCR complex”) is composed of six different type I single-spanning transmembrane proteins: the TCRα and TCRβ chains that form the TCR heterodimer responsible for ligand recognition, and the non-covalently associated CD3γ, CD3δ, CD3ε and ζ chains, which bear cytoplasmic sequence motifs that are tyrosine phosphorylated upon receptor activation and recruit a large number of signalling components (Call et al. 2004, Molecular Immunology 40: 1295-1305).

Both α and β chains of the heterodimeric T cell receptor (TCR) consist of a constant domain and a variable domain. T cells are activated upon TCR recognition of cognate peptide presented by self-MHC molecules, with signal transduction initiated by tyrosine phosphorylated CD3 complexes, leading to T cell proliferation and differentiation.

Rather than eliciting specific T cell responses, which rely on expression by cancer cells of MHC molecules and the presence, generation, transport and display of specific peptide antigens, more recent developments have attempted to combine the advantages of immunotherapy with antibody therapy by engaging all T cells of a patient in a polyclonal fashion via recombinant antibody-based technologies. Bispecific antibodies have been engineered that have a tumor recognition part on the one arm (target-binding arm) whereas the other arm of the molecule has specificity for a T cell antigen (effector-binding arm), a so-called T cell engager (TCE), which often targets CD3. These bispecific antibodies are multitargeting molecules that enhance the patient's immune response to malignant cells. Co-engagement of T cell and tumor cell by the bispecific antibody leads to the formation of a cytolytic synapse between the T cell and the tumor cell, that induces T cell activation and results in tumor cell killing.

While the majority of T cell activating bispecific antibodies target the CD3 complex on the T cell, some bispecific binders that target the constant domain of the αβ T cell receptor have been described in WO 2016/180969 A1. However, one of the main issues with these T cell activating bispecific antibodies is that little cross-reactivity with cynomolgus T cell receptor was observed.

Bispecific antibody constructs have been proposed in multiple formats. For example, bispecific antibody formats may involve the chemical conjugation of two antibodies or fragments thereof (Brennan M. et al. 1985, Science 229(4708): 81-83; Glennie M. J. et al. 1987, J Immunol 139(7): 2367-2375).

Disadvantages of such bispecific antibody formats include, however, high molecular weight and high viscosity at high concentration, making e.g. subcutaneous administration challenging, and in that each binding unit requires the interaction of two variable domains for specific and high affinity binding, having implications on polypeptide stability and efficiency of production. Such bispecific antibody formats may also potentially lead to CMC issues related to poor production efficiency and low titers and/or mispairing of the light chains or mispairing of the heavy chains.

Currently, only one bispecific antibody, Blinatumomab (a BiTE molecule recognizing CD19 and CD3), is on the market for use in the clinic for the treatment of cancer. Although this T cell engaging format was approved in December 2014 for second line treatment by the FDA, many hurdles had to be overcome. The first clinical trials of Blinatumomab were prematurely stopped due to neurologic adverse events, cytokine release syndrome (CRS) and infections on the one hand and the absence of objective clinical responses or robust signs of biological activity on the other hand. CRS is the most important adverse event reported in the first T cell-engaging therapies.

To minimize the risk for adverse events and systemic side effects, such as cytokine storms, utmost care must be taken upon selection of the T cell antigen arm. The latter must bind to the TCR complex in a monovalent fashion and may not trigger T cell signalling in the absence of the targeted cancer cells. Only the specific binding of both arms of the bispecific antibody to their targets (the tumor and the T cell antigen) may trigger the formation of the cytolytic synapses and subsequent killing of the tumor cells.

Non-human primates, such as cynomolgus or rhesus monkeys, are generally considered to be the most suitable animal species for preclinical studies, including efficacy and toxicity studies. To enable assessment of toxicity of a bispecific T cell engaging antibody in non-human primates, good species cross-reactivity of the antibody for human and non-human primate TCR is advisable.

Therefore, there remains a need for additional multispecific T cell engaging formats and, in particular, multispecific T cell engaging formats targeting T cell receptors different from CD3.

Additionally, there is a need for antibody constructs that bind both to a target cell and a T cell with sufficient affinity to induce a cytotoxic response. At the same time, such constructs should not induce a cytotoxic response to non-target cells, i.e. cells that do not express the target antigen or only express it at low levels. Thereby, a balance can be struck between efficacy and safety. It is further desirable that such constructs can be efficiently produced, e.g. in microbial hosts. Moreover, the constructs, when used therapeutically, should exert no or only minimal undesired side effects, e.g. provoked by cytotoxic activity on non-target cells.

### 3 SUMMARY OF THE PRESENT TECHNOLOGY

The present inventors have found novel T cell engager (TCE) immunoglobulin single variable domains (ISVDs) that are capable of specifically targeting and binding the TCR. These TCR binding ISVDs, also referred to as TCEs, can be linked to a moiety able to bind a cell-specific target. Pathologies that are caused by abnormal cells, such as cancer, which present said specific target on their cell surface can be targeted by the immune system through the binding of the TCE ISVD to the T cells. Consequently, T cells are directed to the tumor cells that express said specific target and through this binding of the T cells to the TCE, T cell activation is induced. Once the T cells are activated, effective target cell killing is triggered.

The inventors found that a construct comprising an ISVD targeting TCR according to the present technology and a cell-specific target at the same time leads to efficient T cell-mediated killing of the target expressing cells in vitro. Moreover, such constructs showed only limited activity against cells expressing no or low levels the target. This suggests the possibility of inducing a highly specific T cell-mediated cytotoxic response against specific target cells, while exhibiting a favourable safety profile.

In stability studies with an earlier developed TCR binding ISVD, the inventors observed lack of chemical stability for some of the amino acid residues at certain positions in the ISVD. Specifically, they observed that isomerization occurred to the aspartic acid (D) in position 61 and that tryptophan (W) oxidation occurred in position 99 (Kabat numbering). It was hypothesized that in downstream processing trace levels of transition metals are catalysed by polysorbate, which led to the observed tryptophan oxidation. The inventors made several amino acid substitutions in selected positions of the TCR binding ISVD and observed that these detrimental effects could be diminished or even fully prevented.

Provided herein are TCR binding ISVDs that have potent binding to TCRs of different species and improved chemical stability.

Thus, in a first aspect, the present technology relates to an ISVD that specifically targets a constant domain of a human and/or of a non-human primate T cell receptor present on a T cell, wherein the ISVD comprises three complementarity determining regions (CDR1 to CDR3, respectively), and wherein:
a. the amino acid sequence of CDR1 (according to Kabat) is INFYG (SEQ ID NO: 79);
b. the amino acid sequence of CDR2 (according to Kabat) is HISIGDQTDYAX₁SAKG (SEQ ID NO: 80); and
c. the amino acid sequence of CDR3 (according to Kabat) is LSRIX₂PYDY (SEQ ID NO: 81);
wherein
the amino acid residue X₁ is selected from E, D, N, P, K, R, I, T, H, V, A, Y, L, Q, F, and S; and/or
the amino acid residue X₂ is selected from Y, A, P, D, Q, E, R, F, S, G, T, H, V, K, L, and I.

Another aspect of the present technology relates to an ISVD which specifically binds to a constant domain of a human and/or non-human primate T cell receptor (TCR) present on a T cell, wherein said ISVD comprises 3 complementarity determining regions (CDR1 to CDR3 respectively), wherein
a. the amino acid sequence of CDR1 (according to AbM) is GYVHKINFYG (SEQ ID NO: 82);
b. the amino acid sequence of CDR2 (according to AbM) is HISIGDQTD (SEQ ID NO: 83); and
c. the amino acid sequence of CDR3 (according to AbM) is LSRIX₂PYDY (SEQ ID NO: 84);
and wherein
the amino acid residue at position 61 (according to Kabat) is selected from E, D, N, P, K, R, I, T, H, V, A, Y, L, Q F, and S; and/or
the amino acid residue X₂ is selected from Y, A, P, D, Q, E, R, F, S, G, T, H, V, K, L and I.

In one embodiment, X₁ is selected from E, D, N, P, K, R, I, T, H, V, A, Y, L, Q F, and S.

In an embodiment, X₁ is selected from E or D, such as X₁ is E.

In another embodiment, position 61 (according to Kabat) is selected from E, D, N, P, K, R, I, T, H, V, A, Y, L, Q, F, and S, such as position 61 (according to Kabat) is E or D.

In one embodiment, position 61 (according to Kabat) is E.

In one embodiment, X₂ is selected from Y, A, P, D, Q, E, R, F, S, G, T, H, V, K, L and I. In one embodiment, X₂ is Y, A, Q, F, S, T or H. In one embodiment, X₂ is Y, Q S or T. In one embodiment, X₂ is Y.

In one embodiment, the amino acid residue in the ISVD at position 103 (Kabat numbering) is selected from the group consisting of W, R, A, E, Y, L, H, I, Q, V, K, S, G, P, F, T, such as the amino acid at position 103 (Kabat numbering) is W.

In another embodiment, the present technology provides an ISVD wherein X₁ is E, X₂ is Y and the amino acid residue at position 103 (Kabat numbering) is W; or an ISVD wherein the amino acid residue at position 61 (Kabat numbering) is E, X₂ is Y and the amino acid residue at position 103 (Kabat numbering) is W.

In a further embodiment, the ISVD is a heavy-chain ISVD. In one embodiment, the ISVD is selected from a VHH, a humanized VHH, a camelized VH, a domain antibody, a single domain antibody and a dAb. In one embodiment, the ISVD is selected from a VHH, a humanized VHH and a camelized VH. In one embodiment, the ISVD has a degree of sequence identity with the sequence of SEQ ID NOs: 2-57 of at least 85%, preferably at least 90%, more preferably at least 95%, in which for the purposes of determining the degree of sequence identity, the amino acid residues that form the CDR sequences are disregarded.

A further aspect of the present technology relates to an ISVD, wherein the sequence of the ISVD is

(SEQ ID NO: 85)
X₀VQLVESGGGVVQPGGSLRLSCVASGYVHKINFYGWYRQA
PGKEREKVAHISIGDQTDYAX₁SAKGRFTISRDESKNTVYL
QMNSLRPEDTAAYYCRALSRIX₂PYDYX₃GQGTLVTVSS,

wherein
   a. X₀ is selected from E and D.
   b. X₁ is selected from the group consisting of E, D, N, P, K, R, I, T, H, V, A, Y, L, Q, F, and S;
   c. X₂ is selected from the group consisting of Y, A, P, D, Q, E, R, F, S, G, T, H, V, K, L and I; and
   d. X₃ is selected from the group consisting of W, R, A, E, Y, L, H, I, Q, V, K, S, G, P, F and T.

In one embodiment, X₀ is D. In one embodiment, X₁ is selected from D or E. In one embodiment, X₁ is E. In one embodiment, X₂ is selected from the group consisting of Y, T, S and Q. In one embodiment, X₂ is Y. In one embodiment, X₃ is W.

In another aspect of the present technology, the ISVD according to the present technology is part of a multispecific polypeptide which further comprises a moiety capable of binding to a specific cell-surface target.

In some embodiments of the present technology, the present technology thus provides a polypeptide comprising a first and at least one further ISVD, wherein said first ISVD specifically binds to a constant domain of a human and/or of a non-human primate TCR present on a T cell, and the at least one further ISVD specifically binds to an antigen on a target cell, wherein the first ISVD is an ISVD according to the present technology.

In these embodiments, the amino acid sequence of the first ISVD may have at least 80% sequence identity with at least one of the amino acid sequences of SEQ ID NOs: 2-57, in which for the purposes of determining the degree of sequence identity, the amino acid residues that form the CDR sequences are disregarded. In one embodiment, said first ISVD is selected from the group of amino acid sequences consisting of SEQ ID NOs: 37, 42, 46, 50 and 52, such as SEQ ID NO: 37 or SEQ ID NO: 42.

In another embodiment, the polypeptide may further comprise a third ISVD, which specifically binds to a second antigen on a target cell.

In some embodiments of the present technology, the polypeptide further comprises one or more other groups, residues, moieties, or binding units, optionally linked via one or more peptidic linkers, in which said one or more other groups, residues, moieties or binding units provide the polypeptide with increased half-life, compared to the corresponding polypeptide without said one or more other groups, residues, moieties or binding units. For example, the binding unit can be an ISVD that binds to a (human) serum protein, such as human serum albumin.

Also provided is a nucleic acid molecule encoding the ISVD or polypeptide of the present technology or a vector comprising the nucleic acid.

The present technology also relates to a non-human host or host cell transformed or transfected with the nucleic acid or vector that encodes the ISVD or polypeptide according to the present technology.

The present technology furthermore relates to a composition comprising the ISVD or polypeptide of the present technology, preferably the composition is a pharmaceutical composition.

Further provided is a method for producing the ISVD or polypeptide as disclosed herein, said method at least comprising the steps of:
a. expressing, in a suitable host cell or host organism or in another suitable expression system, a nucleic acid sequence encoding the ISVD or polypeptide; optionally followed by:
b. isolating and/or purifying the ISVD or polypeptide.

Moreover, the present technology relates to the composition or polypeptides for use as a medicament.

In one embodiment of the present technology, the polypeptide or composition is for use in the treatment of a proliferative disease, an inflammatory disease, an infectious disease or an autoimmune disease. In one embodiment of the present technology, said proliferative disease is cancer.

The present technology also provides a method of treatment comprising the step of administering the composition or polypeptides to a subject in need thereof.

In one embodiment of the present technology, the method of treatment is for treating a proliferative disease, an inflammatory disease, an infectious disease, or an autoimmune disease. In one embodiment, said proliferative disease is cancer.

The present technology additionally provides the composition or polypeptides for use in the preparation of a medicament. In one embodiment of the present technology, the medicament is used in the treatment of a proliferative disease, an inflammatory disease, an infectious disease or an autoimmune disease.

In one embodiment of the present technology, said proliferative disease is cancer.

### 4 BRIEF DESCRIPTION OF THE DRAWINGS

FIG. 1 shows the binding by the TCE ISVDs according to the present technology to primary human T cells as determined in flow cytometry (FACS).

FIG. 2 shows the binding by the TCE-CD123-ALB ISVD constructs according to the present technology to primary human T cells as determined in flow cytometry (FACS).

FIGS. 3A-3F show dose-response curves of the TCE-CD123-ALB constructs in the flow cytometry-based human T cell mediated MOLM-13 cell killing assay using an effector to target ratio of 10 to 1 in the presence of 30 μM HSA. Cells were obtained from 2 human donors. Results for donor 1 are shown in FIG. 3A, FIG. 3C and FIG. 3E, while results for donor 2 are shown in FIG. 3B, FIG. 3D and FIG. 3F.

FIGS. 4A-4B show dose-response curves of the TCE-CD123-ALB constructs in the flow cytometry-based human (FIG. 4A) and cyno (FIG. 4B) T cell mediated KG-1a cell killing assay using an effector to target ratio of 10 to 1 in the presence of 30 μM HSA.

FIGS. 5A-5D show dose response curves of the TCE-GPC3-GPC3-ALB constructs in the impedance-based human (FIG. 5A and FIG. 5B) and cynomolgus (FIG. 5C and FIG. 5D) T cell mediated HuH-7 cell killing assay using an effector to target ratio of 15 to 1 in the presence of 30 μM HSA.

FIG. 6 shows the evolution of relative tryptophan oxidation at 25° C., quantified as the sum of peaks with RRT <1.0 in reverse phase chromatography, for both ISVD constructs A022600424 and A022600462.

FIG. 7 shows the evolution of tryptophan oxidation, quantified as the sum of peaks with RRT <1.0 in reverse phase chromatography, for both ISVD constructs A022600424 and A022600462, under forced degradation conditions after 1 day and after 2 weeks.

FIG. 8 shows the results of peptide mapping of ISVD constructs A022600424 and A022600462.

### 5 DETAILED DESCRIPTION OF THE PRESENT TECHNOLOGY

The inventors found in previous studies that the introduction of certain amino acid mutations—and combinations thereof—in the CDRs of ISVD T0170056G05 (disclosed as SEQ ID NO: 50 in WO2016180969) resulted in improved binding to the constant domains of a human TCR and/or of a non-human primate TCR. However, the improved TCR-binding ISVD that was developed, named T017000700 (SEQ ID NO: 1) was shown to have some issues in chemical stability studies related to isomerization and tryptophan oxidation.

Consequently, there remained a need for further improved TCR binding ISVDs.

The present inventors have now found that the introduction of certain amino acid mutations at particular positions within the sequence of the ISVD can remove the issues observed with T017000700, whilst retaining the improved TCR binding with regards to T0170056G05 (disclosed as SEQ ID NO: 50 in WO2016180969).

Amino acid residues will be indicated interchangeably herein according to the standard three-letter or one-letter amino acid code, as mentioned in Table B-1 below.

TABLE B-1
Common amino acids

| 1-Letter | 3-Letter | |
|---|---|---|
| Code | Code | Name |
| A | Ala | Alanine |
| C | Cys | Cysteine |
| D | Asp | Aspartic acid |
| E | Glu | Glutamic acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| X | Xaa | Unspecified |
| Y | Tyr | Tyrosine |

When an amino acid residue is indicated as “X” or “Xaa”, it means that the amino acid residue is unspecified, unless the context requires a more limited interpretation. For example, if the description provides an amino acid sequence of a CDR wherein one (or more) of the amino acid residue(s) is (are) indicated with “X”, the description may further specify which amino acid residue(s) is (can be) present at that specific position of the CDR.

### 5.1 Immunoglobulin Single Variable Domains

The term “immunoglobulin single variable domain” (ISVD), defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody (such as an IgG, IgM, IgA, IgD or IgE molecule; known in the art) or of a Fab fragment, a F(ab′)₂ fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISVD) can for example be a heavy chain ISVD, such as a V_{H}, V_{HH}, including a camelized V_{H} or humanized V_{HH}. Preferably, it is a V_{HH}, including a camelized V_{H} or humanized V_{HH}. Heavy chain ISVDs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb); other single variable domains, or any suitable fragment of any one thereof.

In particular, the immunoglobulin single variable domain may be a NANOBODY® immunoglobulin single variable domain (such as a V_{HH}, including a humanized V_{HH}, or camelized V_{H}) or a suitable fragment thereof. NANOBODY® and NANOBODIES® are registered trademarks of Ablynx N.V.

“V_{HH} domains”, also known as V_{HH}s, V_{HH} antibody fragments, and V_{HH} antibodies, have originally been described as the antigen binding immunoglobulin variable domain of “heavy chain antibodies” (i.e., of “antibodies devoid of light chains”; Hamers-Casterman et al. 1993, Nature 363: 446-448). The term “V_{HH} domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans 2001 (Reviews in Molecular Biotechnology 74: 277-302).

Typically, the generation of immunoglobulins involves the immunization of experimental animals, fusion of immunoglobulin producing cells to create hybridomas and screening for the desired specificities. Alternatively, immunoglobulins can be generated by screening of naïve or synthetic libraries e.g. by phage display.

The generation of immunoglobulin sequences has been described extensively in various publications, among which WO 94/04678, Hamers-Casterman et al. 1993 and Muyldermans et al. 2001 can be exemplified. In these methods, camelids are immunized with the target antigen in order to induce an immune response against said target antigen. The repertoire of VHHs obtained from said immunization is further screened for VHHs that bind the target antigen.

In these instances, the generation of immunoglobulins requires purified antigen for immunization and/or screening. Antigens can be purified from natural sources, or in the course of recombinant production.

Immunization and/or screening for immunoglobulin sequences can be performed using peptide fragments of such antigens.

The present technology may use immunoglobulin sequences of different origin, comprising mouse, rat, rabbit, donkey, human and camelid immunoglobulin sequences. The technology also includes fully human, humanized, or chimeric sequences. For example, the technology comprises camelid immunoglobulin sequences and humanized camelid immunoglobulin sequences, or camelized domain antibodies, e.g. camelized dAb as described by Ward et al (see for example WO 94/04678 and Davies and Riechmann 1994 and 1996). Moreover, the technology also uses fused immunoglobulin sequences, e.g. forming a multivalent and/or multispecific construct (for multivalent and multispecific polypeptides containing one or more V_{HH} domains and their preparation, reference is also made to Conrath et al. 2001, J. Biol. Chem. 276 (10): 7346-7350, as well as to for example WO 96/34103 and WO 99/23221), and immunoglobulin sequences comprising tags or other functional moieties, e.g. toxins, labels, radiochemicals, etc., which are derivable from the immunoglobulin sequences of the present technology.

A “humanized V_{HH}” comprises an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example based on the further description herein and the prior art (e.g. WO 2008/020079). Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example based on the further description herein and the prior art (e.g. WO 2008/020079). Such “camelizing” substitutions are preferably inserted at amino acid positions that form and/or are present at the V_{H}-V_{L} interface, and/or at the so-called Camelidae hallmark residues, as defined herein (see for example WO 94/04678 and Davies and Riechmann 1994 and 1996, supra). Preferably, the V_{H} sequence that is used as a starting material or starting point for generating or designing the camelized V_{H} is preferably a V_{H} sequence from a mammal, more preferably the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

A preferred structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

As further described in paragraph q) on pages 58 and 59 of WO 08/020079, the amino acid residues of an immunoglobulin single variable domain can be numbered according to the general numbering for V_{H} domains given by Kabat et al. (“Sequence of proteins of immunological interest”, US Public Health Services, NIH Bethesda, MD, Publication No. 91), as applied to V_{HH} domains from Camelids in the article of Riechmann and Muyldermans 2000 (J. Immunol. Methods 240 (1-2): 185-195; see for example FIG. 2 of this publication). It should be noted that—as is well known in the art for V_{H} domains and for V_{HH} domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a V_{H} domain and a V_{HH} domain will usually be in the range of from 110 to 120, often between 112 and 115. It should however be noted that smaller and longer sequences may also be suitable for the purposes described herein.

In the present application, unless indicated otherwise, CDR sequences were determined according to Kabat (Martin 2010, In: Kontermann and Dubel (eds.), Antibody Engineering Vol. 2, Springer Verlag Heidelberg Berlin, Chapter 3, pp. 33-51). According to this method, FR1 of an immunoglobulin single variable domain comprises the amino acid residues at positions 1-30, CDR1 of an immunoglobulin single variable domain comprises the amino acid residues at positions 31-35, FR2 of an immunoglobulin single variable domain comprises the amino acids at positions 36-49, CDR2 of an immunoglobulin single variable domain comprises the amino acid residues at positions 50-65, FR3 of an immunoglobulin single variable domain comprises the amino acid residues at positions 66-94, CDR3 of an immunoglobulin single variable domain comprises the amino acid residues at positions 95-102, and FR4 of an immunoglobulin single variable domain comprises the amino acid residues at positions 103-113.

Determination of CDR regions may also be done according to different methods. In the present application, CDR sequences were also determined according to the AbM definition as described in Martin 2010 (In: Kontermann and Dubel (Eds.) 2010, Antibody Engineering, vol 2, Springer Verlag Heidelberg Berlin, Chapter 3, pp. 33-51). According to this method, FR1 comprises the amino acid residues at positions 1-25, CDR1 comprises the amino acid residues at positions 26-35, FR2 comprises the amino acids at positions 36-49, CDR2 comprises the amino acid residues at positions 50-58, FR3 comprises the amino acid residues at positions 59-94, CDR3 comprises the amino acid residues at positions 95-102, and FR4 comprises the amino acid residues at positions 103-113.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are preferably (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or V_{HH} sequence). In one particularly preferred aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISVD sequence used in the technology may contain one or more of hallmark residues (as defined herein), such that the ISVD sequence is a V_{HH}, including a humanized V_{HH} or camelized V_{H}. Some preferred, but non-limiting examples of (suitable combinations of) such framework sequences will become clear from the further disclosure herein.

Again, as generally described herein for the immunoglobulin sequences, it is also possible to use suitable fragments (or combinations of fragments) of any of the foregoing, such as fragments that contain one or more CDR sequences, suitably flanked by and/or linked via one or more framework sequences (for example, in the same order as these CDR's and framework sequences may occur in the full-sized immunoglobulin sequence from which the fragment has been derived).

However, it should be noted that the technology is not limited as to the origin of the ISVD sequence (or of the nucleotide sequence used to express it), nor as to the way that the ISVD sequence or nucleotide sequence is (or has been) generated or obtained. Thus, the ISVD sequences may be naturally occurring sequences (from any suitable species) or synthetic or semi-synthetic sequences. In a specific but non-limiting aspect, the ISVD sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

As described above, an ISVD may be an ISVD or a suitable fragment thereof. For a general description of ISVDs, reference is made to the further description below, as well as to the prior art cited herein. In this respect, it should however be noted that this description and the prior art mainly described ISVDs of the so-called “V_{H}3 class” (i.e. ISVDs with a high degree of sequence homology to human germline sequences of the V_{H}3 class such as DP-47, DP-51, or DP-29). It should however be noted that the technology in its broadest sense can generally use any type of ISVD, and for example also uses the ISVDs belonging to the so-called “V_{H}4 class” (i.e. ISVDs with a high degree of sequence homology to human germline sequences of the V_{H}4 class such as DP-78), as for example described in WO 2007/118670.

Generally, ISVDs (in particular V_{HH} sequences, including (partially) humanized V_{HH} sequences and camelized V_{H} sequences) can be characterized by the presence of one or more “Hallmark residues” (as described herein) in one or more of the framework sequences (again as further described herein). Thus, generally, an ISVD can be defined as an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, an ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, an ISVD can be an immunoglobulin sequence with the (general) structure

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the amino acid residues at positions 11, 37, 44, 45, 47, 83, 84, 103, 104 and 108 according to the Kabat numbering are selected from the Hallmark residues mentioned in Table A-0 below.

TABLE A-0
Hallmark Residues in ISVDs

| Position | Human V_{H}3 | Hallmark Residues |
|---|---|---|
| 11 | L, V; predominantly L | L, S, V, M, W, F, T, Q, E, A, |
| | | R, G, K, Y, N, P, I; preferably L |
| 37 | V, I, F; usually V | F⁽¹⁾, Y, V, L, A, H, S, I, W, C, |
| | | N, G, D, T, P, preferably F⁽¹⁾ or Y |
| 44⁽⁸⁾ | G | E⁽³⁾, Q⁽³⁾, G⁽²⁾, D, A, K, R, L, P, |
| | | S, V, H, T, N, W, M, I; preferably G⁽²⁾, |
| | | E⁽³⁾ or Q⁽³⁾; most preferably G⁽²⁾ or Q⁽³⁾ |
| 45⁽⁸⁾ | L | L⁽²⁾, R⁽³⁾, P, H, F, G, Q, S, E, T, |
| | | Y, C, I, D, V; preferably L⁽²⁾ or R⁽³⁾ |
| 47⁽⁸⁾ | W, Y | F⁽¹⁾, L⁽¹⁾ or W⁽²⁾ G, I, S, A, V, M, R, |
| | | Y, E, P, T, C, H, K, Q, N, D; preferably |
| | | W⁽²⁾ , L⁽¹⁾ or F⁽¹⁾ |
| 83 | R or K; usually R | R, K⁽⁵⁾, T, E⁽⁵⁾, Q, N, S, I, V, G, M, |
| | | L, A, D, Y, H; preferably K or R; most |
| | | preferably K |
| 84 | A, T, D; | P⁽⁵⁾, S, H, L, A, V, I, T, F, D, R, Y, |
| | predominantly A | N, Q, G, E; preferably P |
| 103 | W | W⁽⁴⁾, R⁽⁶⁾, G, S, K, A, M, Y, L, F, T, |
| | | N, V, Q, P⁽⁶⁾, E, C; preferably W |
| 104 | G | G, A, S, T, D, P, N, E, C, L; preferably G |
| 108 | L, M or T; | Q, L⁽⁷⁾, R, P, E, K, S, T, M, A, H; |
| | predominantly L | preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q in (non-humanized) V_{HH} sequences that also contain a W at 103.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

The technology inter alia uses ISVDs that can bind to the constant domain of a TCR. In the context of the present technology, “binding to” a certain target molecule has the usual meaning in the art as understood in the context of antibodies and their respective antigens.

Accordingly, the target molecules of the ISVDs used in the technology are the constant domain of the TCR.

Binding to TCR can be achieved, for example, by binding to the TCRalpha subunit and/or the TCRbeta subunit. An example is mammalian TCR. While human TCR is preferred, the versions from other species are also amenable to the present technology, for example TCR from mice, rats, rabbits, cats, dogs, goats, sheep, horses, pigs, non-human primates, such as cynomolgus monkeys (also referred to herein as “cyno”), or camelids, such as llama or alpaca.

The sequences of the TCR-α/β constant domains of human and cyno origin are provided in Table A-1 (SEQ ID NO: 106 and 108 for the constant domain of TCR a from human and cyno origin, respectively; SEQ ID NO: 107 and 109 for the constant domain of TCR P from human and cyno origin, respectively). The origin of each of these sequences, as expressed by a UniProt or Genbank files identifier, is listed for each of the aforementioned sequences in Table A-1. In house sequencing confirmed that the amino acid sequences originally derived from rhesus origin, were identical to those from cyno origin.

In one embodiment, the ISVD specifically binds to the constant domain of a human T cell receptor α (TCR-α) (SEQ ID NO: 106) and/or the constant domain of the human T cell receptor β (TCR-β) (SEQ ID NO: 107), or polymorphic variants or isoforms thereof.

In one embodiment, the ISVD specifically binds to the constant domain of a non-human primate TCR. In one embodiment, the non-human primate TCR is a macaque or rhesus TCR. In one embodiment, the macaque or rhesus TCR comprises the constant domain of a TCR-α of SEQ ID NO: 108 and/or of a TCR-β of SEQ ID NO: 109, or polymorphic variants or isoforms thereof.

Isoforms are alternative protein sequences that can be generated from the same gene by a single or by the combination of biological events such as alternative promoter usage, alternative splicing, alternative initiation and ribosomal frameshifting, all as known in the art.

TABLE A-1
Amino acid sequences related to TCR (“ID” refers
to the given SEQ ID NO as used herein)

| ID | Name | Amino acid sequence |
|---|---|---|
| 106 | Human TCR alpha | PNIQNPDPAVYQLRDSKSSDKSVCLFTDFDSQTNVSQSKDSDVYIT |
| | constant domain | DKTVLDMRSMDFKSNSAVAWSNKSDFACANAFNNSIIPEDTFFPSP |
| | (derived from P01848) | ESSCDVKLVEKSFETDTNLNFQNLSVIGFRILLLKVAGFNLLMTLR |
| | | LWSS |
| 107 | Human TCR beta | EDLNKVFPPEVAVFEPSEAEISHTQKATLVCLATGFFPDHVELSW |
| | constant domain | WVNGKEVHSGVSTDPQPLKEQPALNDSRYALSSRLRVSATFWQNP |
| | (derived from P01850) | RNHFRCQVQFYGLSENDEWTQDRAKPVTQIVSAEAWGRADCGFTS |
| | | VSYQQGVLSATILYEILLGKATLYAVLVSALVLMAMVKRKDF |
| 108 | Rhesus TCR alpha | MLLLLVLVLEVIFTLGGTRAQSVTQLDSQVSVSEGVPVLLRCNYSSS |
| | (AEA41863) | FSPYLFWYVQYPNQGLQLLLKYTSGTTLVKGINGFEAEFKKSETSFH |
| | | LTKASAHVSDAAEYFCALARGALVFGKGTRLSVIPNIQNPDPAVYQ |
| | | LRGSKSNDTSVCLFTDFDSVMNVSQSKDSDVHITDKTVLDMRSM |
| | | DFKSNGAVAWSNKSDFACTSAFKDSVIPADTFFPGTESVCDANLV |
| | | EKSFETDMNLNFQNLSVIGFRILLLKVAGFNLLMTLRLWSS |
| 109 | Rhesus TCR beta | MGFWTLCCVSFCILVAKHTDAGVIQLPRHEVTEMGKEVTLRCEPIS |
| | (AEA41864) | GHSSLFWYRQTMMRGLEFLIYFNNKSPIDDSGMPKDRFSATMPD |
| | | ASFSTLKIQPSEPRDSAVYFCASTPGQGREKLFFGSGTQLSVLEDLK |
| | | KVFPPKVAVFEPSEAEISHTQKATLVCLATGFYPDHVELSWWVNG |
| | | KEVHSGVSTDPQPLKEQPALEDSRYCLSSRLRVSATFWHNPRNHF |
| | | RCQVQFYGLSEDDEWTEDRDKPITQKISAEVWGRADCGFTSVSY |
| | | QQGVLSATILYEILLGKATLYAVLVSALMLMAMVKRKDF |
| 110 | Human TCR-zipper: | MNMRPVTSSVLVLLLMLRRSNGQLLEQSPQFLSIQEGENLTVYCN |
| | alpha chain | SSSVFSSLQWYRQEPGEGPVLLVTVVTGGEVKKLKRLTFQFGDARK |
| | | DSSLHITAAQPGDTGLYLCAGAGSQGNLIFGKGTKLSVKPNIQNPD |
| | | PAVYQLRDSKSSDKSVCLFTDFDSQTNVSQSKDSDVYITDKSVLDM |
| | | RSMDFKSNSAVAWSNKSDFACANAFNNSIIPEDTFFPSPESSCSSA |
| | | DLVPRGSTTAPSAQLKKKLQALKKKNAQLKWKLQALKKKLAQEQK |
| | | LISEEDL |
| 111 | Human TCR-zipper: | MSNTVLADSAWGITLLSWVTVFLLGTSSADGGITQSPKYLFRKEG |
| | beta chain | QNVTLSCEQNLNHDAMYWYRQDPGQGLRLIYYSQIVNDFQKGDI |
| | | AEGYSVSREKKESFPLTVTSAQKNPTAFYLCASSSRSSYEQYFGPGT |
| | | RLTVTEDLKNVFPPEVAVFEPSEAEISHTQKATLVCLATGFYPDHVE |
| | | LSWWVNGKEVHSGVSTDPQPLKEQPALNDSRYSLSSRLRVSATF |
| | | WQNPRNHFRCQVQFYGLSENDEWTQDRAKPVTQIVSAEAWGR |
| | | ADCSSADLVPRGSTTAPSAQLEKELQALEKENAQLEWELQALEKEL |
| | | AQTGHHHHHHHHHH |
| 112 | Cyno TCR-zipper: alpha | MLLITSVLVLWMQLSQVNGQQIMQIPQYQHVQEGEDFTTYCNSS |
| | chain (derived from | TTLSNIQWYKQRPGGHPVFLIMLVKSGEVKKQKRLIFQFGEAKKNS |
| | AEA41865) | SLHITATQTTDVGTYFCATTGVNNLFFGTGTRLTVLPYIQNPDPAV |
| | | YQLRGSKSNDTSVCLFTDFDSVMNVSQSKDSDVHITDKTVLDMRS |
| | | MDFKSNGAVAWSNKSDFACTSAFKDSVIPADTFFPSPESSCSSADL |
| | | VPRGSTTAPSAQLKKKLQALKKKNAQLKWKLQALKKKLAQEQKLIS |
| | | EEDL |
| 113 | Cyno TCR-zipper: beta | MSIGLLCCAALSLLWAGPVNAGVTQTPKFQVLKTGQSMTLQCAQ |
| | chain (derived from | DMNHDYMYWYRQDPGMGLRLIHYSVGEGSTEKGEVPDGYNVT |
| | AEA41866 and | RSNTEDFPLRLESAAPSQTSVYFCASSYWTGRSYEQYFGPGTRLTVI |
| | AEA41868) | EDLKKVFPPKVAVFEPSEAEISHTQKATLVCLATGFYPDHVELSWW |
| | | VNGKEVHSGVSTDPQPLKEQPALEDSRYSLSSRLRVSATFWHNPR |
| | | NHFRCQVQFYGLSEDDEWTEDRDKPITQKISAEAWGRADCSSAD |
| | | LVPRGSTTAPSAQLEKELQALEKENAQLEWELQALEKELAQTGHH |
| | | HHHHHHHH |
| 114 | Human TCR alpha | PNIQNPDPAVYQLRDSKSSDKSVCLFTDFDSQTNVSQSKDSDVYIT |
| | constant domain | DKTVLDMRSMDFKSNSAVAWSNKSDFACANAFNNSIIPEDTFFPS |
| | (derived from P01848) | PESSC |
| 115 | Human TCR beta | EDLNKVFPPEVAVFEPSEAEISHTQKATLVCLATGFFPDHVELSWW |
| | constant domain | VNGKEVHSGVSTDPQPLKEQPALNDSRYALSSRLRVSATFWQNPR |
| | (derived from P01850) | NHFRCQVQFYGLSENDEWTQDRAKPVTQIVSAEAWGRADC |
| 116 | cynomolgus TCR-alpha | PYIQNPDPAVYQLRGSKSNDTSVCLFTDFDSVMNVSQSKDSDVHI |
| | constant domain | TDKTVLDMRSMDFKSNGAVAWSNKSDFACTSAFKDSVIPADTFF |
| | (derived from | PSPESSC |
| | AEA41865) | |
| 117 | cynomolgus TCR-beta | EDLKKVFPPKVAVFEPSEAEISHTQKATLVCLATGFYPDHVELSWW |
| | constant domain | VNGKEVHSGVSTDPQPLKEQPALEDSRYSLSSRLRVSATFWHNPR |
| | (derived from | NHFRCQVQFYGLSEDDEWTEDRDKPITQKISAEAWGRADC |
| | AEA41868) | |

Thus, in a first aspect the present technology relates to an ISVD which specifically binds to a constant domain of a human and/or non-human primate T cell receptor (TCR) present on a T cell, wherein said ISVD essentially consists of 4 framework regions (FR1 to FR4 respectively) and 3 complementarity determining regions (CDR1 to CDR3 respectively), wherein
a. the amino acid sequence of CDR1 (according to Kabat) is INFYG (SEQ ID NO: 79);
b. the amino acid sequence of CDR2 (according to Kabat) is HISIGDQTDYAX₁SAKG (SEQ ID NO: 80); and
c. the amino acid sequence of CDR3 (according to Kabat) is LSRIX₂PYDY (SEQ ID NO: 81);
wherein
the amino acid residue X₁ is selected from E, D, N, P, K, R, I, T, H, V, A, Y, L, Q, F, and S; and/or
the amino acid residue X₂ is selected from Y, A, P, D, Q, E, R, F, S, G, T, H, V, K, L and I.

The ISVD sequence may also be represented using the AbM definition for CDRs as follows. An ISVD, which specifically binds to a constant domain of a human and/or non-human primate T cell receptor (TCR) present on a T cell, wherein said ISVD essentially consists of 4 framework regions (FR1 to FR4 respectively) and 3 complementarity determining regions (CDR1 to CDR3 respectively), wherein
a. the amino acid sequence of CDR1 (according to AbM) is GYVHKINFYG (SEQ ID NO: 82);
b. the amino acid sequence of CDR2 (according to AbM) is HISIGDQTD (SEQ ID NO: 83); and
c. the amino acid sequence of CDR3 (according to AbM) is LSRIX₂PYDY (SEQ ID NO: 84);
and wherein
the amino acid residue at position 61 (according to Kabat) is selected from E, N, P, K, R, I, T, H, V, A, Y, L, Q, F, and S; and/or
the amino acid residue X₂ is selected from Y, A, P, D, Q, E, R, F, S, G, T, H, V, K, L and I.

The inventors found that an ISVD with the CDRs according to the present technology had potent TCR binding abilities and did not have isomerization or tryptophan oxidation at the relevant positions 61 and 99 (Kabat numbering) respectively. This means that the molecule is both effective at engaging T cells as well as being chemically stable.

In an embodiment of the present technology, the amino acid sequence of the ISVD specifically binding to human TCR may exhibit a sequence identity of more than 85%, such as more than 90%, more than 95% or more than 99%, with any of SEQ ID NOs: 2-57, wherein the CDRs are as defined herein.

In a further embodiment, amino acid sequence of the ISVD specifically binding to human TCR may exhibit a sequence identity of more than 85%, preferably at least 90%, more preferably at least 95%. Preferably, the sequence comprises SEQ ID NO: 37, SEQ ID NO: 42, SEQ ID NO: 46, SEQ ID NO: 50 or SEQ ID NO: 52, such as SEQ ID NO: 37 or SEQ ID NO: 42.

In another embodiment of the present technology, the amino acid sequence of the ISVD specifically binding to human TCR may exhibit a sequence identity of more than 85%, such as more than 90%, more than 95% or more than 99%, with any of SEQ ID NOs: 2-57, in which for the purposes of determining the degree of sequence identity, the amino acid residues that form the CDR sequences are disregarded.

In a further embodiment, amino acid sequence of the ISVD specifically binding to human TCR may exhibit a sequence identity of more than 85%, preferably at least 90%, more preferably at least 95%, in which for the purposes of determining the degree of sequence identity, the amino acid residues that form the CDR sequences are disregarded. Preferably, the sequence comprises SEQ ID NO: 37, SEQ ID NO: 42, SEQ ID NO: 46, SEQ ID NO: 50 or SEQ ID NO: 52, such as SEQ ID NO: 37 or SEQ ID NO: 42.

When the ISVD exhibits a sequence identity of more than 85%, such as more than 90%, more than 95% or more than 99%, with any of SEQ ID NOs: 2-57, the ISVD preferably exhibits at least half the binding affinity, more preferably at least the same binding affinity to human TCR compared to one of the ISVDs set forth in Table A-4, wherein the binding affinity is measured using the same method, such as surface plasmon resonance (SPR).

Additionally, when the ISVD exhibits a sequence identity of more than 85%, such as more than 90%, more than 95% or more than 99%, with any of SEQ ID NOs: 2-57, the ISVD preferably exhibits at least half of the potency in T cell mediated target cell killing, more preferably at least the same potency in T cell mediated target cell killing compared to one of the ISVDs set forth in Table A-4, wherein the T cell mediated target cell killing is measured using the same method, such as a flow cytometry-based T cell mediated cell killing assay or impedance-based T cell mediated killing assay.

The percentage of “sequence identity” between a first amino acid sequence and a second amino acid sequence may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (i.e. at a single position).

Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

An “amino acid difference” as used herein refers to a deletion, insertion, or substitution of a single amino acid residue vis-h-vis a reference sequence, and preferably is a substitution.

In one embodiment of the present technology, amino acid substitutions are conservative substitutions. Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, lie, Val and Cys; and (e) aromatic residues: Phe, Tyr and Trp.

In another embodiment of the present technology, the conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In an embodiment of the present technology, the entire amino acid sequence of the ISVD is represented by SEQ ID NO: 85, which is X₀VQLVESGGGVVQPGGSLRLSCVASGYVHKINFYGWYRQAPGKEREKVAHISIGDQTDYAX₁SAKGR FTISRDESKNTVYLQMNSLRPEDTAAYYCRALSRIX₂PYDYX₃GQGTLVTVSS, wherein
a. X₀ is selected from E and D.
b. X₁ is selected from the group consisting of E, D N, P, K, R, I, T, H, V, A, Y, L, Q, F, and S;
c. X₂ is selected from the group consisting of Y, A, P, D, Q, E, R, F, S, G, T, H, V, K, L and I; and
d. X₃ is selected from the group consisting of W, R, A, E, Y, L, H, I, Q, V, K, S, G, P, F and T.

In an embodiment of the present technology, X₀ is D, and/or X₁ is E, and/or X₂ is any of Y, T, S or Q, and/or X₃ is W.

In another embodiment of the present technology X₀ is E, and/or X₁ is E, and/or X₂ is any of Y, T, S or Q, and/or X₃ is W.

In one embodiment of the present technology X₀ is E, and/or X₁ is E, and/or X₂ is T, and/or X₃ is W.

In a further embodiment of the present technology, X₀ is D, and/or X₁ is E, and/or X₂ is T, and/or X₃ is W.

In another embodiment of the present technology, X₀ is E, and/or X₁ is E, and/or X₂ is S, and/or X₃ is W.

In an embodiment of the present technology, X₀ is D, and/or X₁ is E, and/or X₂ is S, and/or X₃ is W.

In one embodiment of the present technology, X₀ is E, and/or X₁ is E, and/or X₂ is Q, and/or X₃ is W.

In another embodiment of the present technology, X₀ is D, and/or X₁ is E, and/or X₂ is Q, and/or X₃ is W.

In a further embodiment of the present technology, X₀ is E, and/or X₁ is E, and/or X₂ is Y, and/or X₃ is W.

In an embodiment of the present technology, X₀ is D, and/or X₁ is E, and/or X₂ is Y, and/or X₃ is W.

The inventors found that substituting the amino acid in the first position of the sequence (Kabat numbering) from E to D could prevent pyroglutamate formation, while the efficacy of the ISVD was unaffected.

In another embodiment of the present technology, the ISVD according to the present technology has the amino acid sequence of any one of SEQ ID NOs: 37, 42, 46, 50 and 52.

In a further embodiment of the present technology, the ISVD has the amino acid sequence of SEQ ID NO: 37 or SEQ ID NO: 42.

The inventors observed that ISVDs according to the present technology with an E in position 61 and a Y, A, S or H in position 99 (Kabat numbering) had a similar or higher melting temperature (Tm), a higher temperature for onset of aggregation (Tagg) and a reduction in the oligomeric fraction when compared to the reference ISVD T017000700. For ISVDs with an E in position 61 and a Q or T in position 99 (Kabat numbering) there was no significant decrease in Tm, while maintaining or having a higher Tagg and a similar or reduced oligomeric fraction (A % oligo).

As such, in an embodiment, the ISVD according to the present technology or an ISVD that exhibits a sequence identity of more than 90%, such as more than 95% or more than 99%, with any of SEQ ID NOs: 2-57, has a melting temperature (Tm) of at least 71° C., at least 72° C., preferably at least 72.5° C., more preferably at least 73° C.

In another embodiment the ISVD according to the present technology or an ISVD that exhibits a sequence identity of more than 90%, such as more than 95% or more than 99%, with any of SEQ ID NOs: 2-57, has a temperature of aggregation onset (Tagg) of at least 67° C., at least 68° C., at least 69° C., at least 71° C., preferably at least 72° C., more preferably at least 73° C.

In yet a further embodiment, the ISVD according to the present technology or an ISVD that exhibits a sequence identity of more than 90%, such as more than 95% or more than 99%, with any of SEQ ID NOs: 2-57, has an oligomerization fraction (A % oligo) of less than 0.5%, preferably less than 0.4%, more preferably less than 0.3%, even more preferably less than 0.2%, or even less than 0.1%.

A list of generated ISVDs can be found in Table A-4. Additionally, combinations of CDR sequences of the generated ISVDs can be found in Table A-5.

In an embodiment of the present technology, the TCE ISVD has a CDR1, CDR2 and CDR3 sequence selected from the CDR1, CDR2 and CDR3 sequences presented in Table A-5.

In another embodiment of the present technology, the TCE ISVD has a CDR1, CRD2 and CDR3 sequence selected from the combination of CDR sequences presented in the same row in Table A-5.

### 5.2 Multispecific Polypeptides

The inventors found that the introduction of certain amino acid mutations—and combinations thereof—in the CDRs of ISVD T0170056G05 (disclosed as SEQ ID NO: 50 in WO2016180969) resulted in improved binding to the constant domains of a human TCR and/or of a non-human primate TCR. Additional amino acid mutations at specific positions within the sequence of the ISVD could furthermore improve the chemical stability of the ISVD, by minimizing or even preventing isomerization and tryptophan oxidation.

Therefore, in an aspect of the present technology, a polypeptide is provided comprising a first ISVD capable of specifically binding to a constant domain of a human and/or non-human primate T cell receptor (TCR) present on a T cell and a second ISVD capable of specifically binding to a first antigen on a target cell, wherein said first antigen is different from said TCR, and wherein said target cell is different from said T cell, wherein said first and second ISVD essentially consist of 4 framework regions (FR1 to FR4 respectively) and 3 complementarity determining regions (CDR1 to CDR3 respectively), and wherein the first ISVD is an ISVD according to the present technology.

In this multispecific polypeptide, the CDR regions are as defined herein (see item 5.1). The inventors found that a polypeptide comprising a first ISVD with the CDRs according to the present technology had potent TCR binding abilities and did not have isomerization ortryptophan oxidation at the relevant positions 61 and 99 (Kabat numbering) respectively.

In an embodiment, the amino acid sequence of the first ISVD has at least 80% sequence identity with at least one of the amino acid sequence of any of SEQ ID NOs: 2-57, preferably at least 85%, more preferably at least 90%, more preferably at least 95%, in which the sequence of the CDR regions is as defined herein, preferably wherein the sequence of the first ISVD comprises SEQ ID NO: 37, SEQ ID NO: 42, SEQ ID NO: 46, SEQ ID NO: 50 or SEQ ID NO: 52.

In an embodiment, the amino acid sequence of the first ISVD has at least 80% sequence identity with at least one of the amino acid sequence of any of SEQ ID NOs: 2-57, preferably at least 85%, more preferably at least 90%, more preferably at least 95%, in which for the purposes of determining the degree of sequence identity, the amino acid residues that form the CDR sequences are disregarded, preferably wherein the sequence of the first ISVD comprises SEQ ID NO: 37, SEQ ID NO: 42, SEQ ID NO: 46, SEQ ID NO: 50 or SEQ ID NO: 52.

In another embodiment, the first ISVD has at least 80% sequence identity with the amino acid sequence of any of SEQ ID NOs: 32, 33 and/or 35-57, preferably at least 85%, more preferably at least 90%, more preferably at least 95%, in which the sequence of the CDR regions is as defined herein, and wherein preferably the sequence comprises SEQ ID NO: 37, SEQ ID NO: 42, SEQ ID NO: 46, SEQ ID NO: 50 or SEQ ID NO: 52.

In another embodiment, the first ISVD has at least 80% sequence identity with the amino acid sequence of any of SEQ ID NOs: 32, 33 and/or 35-57, preferably at least 85%, more preferably at least 90%, more preferably at least 95%, in which for the purposes of determining the degree of sequence identity, the amino acid residues that form the CDR sequences are disregarded, and wherein preferably the sequence comprises SEQ ID NO: 37, SEQ ID NO: 42, SEQ ID NO: 46, SEQ ID NO: 50 or SEQ ID NO: 52.

In a further embodiment, the first ISVD comprises or consists of SEQ ID NO: 37 or SEQ ID NO: 42.

In yet another embodiment the first ISVD comprises or consists of SEQ ID NO: 46, SEQ ID NO: 50 or SEQ ID NO: 52.

As has been shown in the Examples further provided herein, the substitution of particular amino acid residues at positions 61 and 99 in T017000700 (SEQ ID NO: 1) did not affect the affinity of the ISVD to the constant domain of a human and/or a non-human primate TCR, while the resulting variants had a better chemical stability as compared to the previously developed T017000700 (SEQ ID NO: 1). T017000978 (SEQ ID NO: 37) and T017000991 (SEQ ID NO: 42) were found to be particularly potent and stable. Additionally, T017000995 (SEQ ID NO: 46), T017000999 (SEQ ID NO: 50) and T017001001 (SEQ ID NO: 52) showed a lower affinity for TCR, but maintained high potency in cell killing, suggesting that they may have better biodistribution and thus potential to have a more target-specific effect.

In an embodiment of the present technology, the polypeptide is at least bispecific, but can also be e.g., trispecific, tetraspecific, pentaspecific, etc. Moreover, the polypeptide is at least bivalent, but can also be e.g., trivalent, tetravalent, pentavalent, hexavalent, etc.

The terms “bispecific”, “trispecific”, “tetraspecific”, “pentaspecific”, etc., all fall under the term “multispecific” and refer to binding to two, three, four, five, etc., different target molecules, respectively.

The terms “bivalent”, “trivalent”, “tetravalent”, “pentavalent”, “hexavalent”, etc. all fall under the term “multivalent” and indicate the presence of two, three, four, five, six, etc., binding units/building blocks, respectively, such as ISVDs.

For example, the polypeptide may be bispecific-bivalent, such as a polypeptide comprising or consisting of two ISVDs, wherein one ISVD specifically binds to the constant domain of a human and/or non-human primate TCR on a T cell and one ISVD specifically binds to a cell-surface specific target antigen, wherein the TCR and target antigen are preferably human.

The polypeptide may also be bispecific-trivalent, such as a polypeptide comprising or consisting of three ISVDs, wherein two ISVDs specifically bind to the same cell-surface specific target antigen and one ISVD specifically binds to the constant domain of a human and/or non-human primate TCR on a T cell.

In another example, the polypeptide may be trispecific-trivalent, such a polypeptide comprising or consisting of three ISVDs, wherein one ISVD specifically binds to the constant domain of a human and/or a non-human primate TCR on a T cell, one ISVD specifically binds to a first antigen on a target cell, and one ISVD specifically binds to a second antigen on the same target cell.

In yet another example, the trispecific-trivalent polypeptide, next to one ISVD that specifically binds to the constant domain of a human and/or a non-human primate TCR on a T cell and one ISVD that specifically binds to a first antigen on a target cell, comprises one ISVD that specifically binds to human serum albumin.

Further examples of multispecific-multivalent polypeptides will be clear for the skilled person based on the disclosure herein.

Such a polypeptide may at the same time be biparatopic, for example if two ISVDs bind two different epitopes of the target antigen.

The term “biparatopic” refers to binding to two different parts (e.g., epitopes) of the same target molecule.

The components, preferably ISVDs, of said multispecific-multivalent polypeptides described herein may be linked to each other by one or more suitable linkers, such as peptidic linkers.

The use of linkers to connect two or more (poly)peptides is well known in the art.

One frequently used class of peptidic linkers are known as the “Gly-Ser” or “GS” linkers. These are linkers that essentially consist of glycine (G) and serine (S) residues, and usually comprise one or more repeats of a peptide motif such as the GGGGS (SEQ ID NO: 86) motif (for example, exhibiting the formula (Gly-Gly-Gly-Gly-Ser)ₙ in which n may be 1, 2, 3, 4, 5, 6, 7 or more). Some often-used examples of such GS linkers are 9GS linkers (GGGGSGGGS, SEQ ID NO: 87) 15GS linkers (n=3) and 35GS linkers (n=7). Reference is for example made to Chen et al. 2013 (Adv. Drug Deliv. Rev. 65(10): 1357-1369) and Klein et al. 2014 (Protein Eng. Des. Sel. 27 (10): 325-330). In the polypeptide(s) disclosed herein, the use of 5GS and 9GS linkers to link the components of the polypeptide to each other is preferred. Preferably, a linker of less than 10 amino acids, is used to link a first ISVD capable of specifically binding to TCR to a second ISVD capable of specifically binding to a cell-surface specific target antigen.

Examples of suitable linkers are given in Table A-2 below.

TABLE A-2
Linker sequences (“ID” refers to
the SEQ ID NO as used herein)

| Name | ID | Amino acid sequence |
|---|---|---|
| 3A linker | 136 | AAA |
| 5GS linker | 86 | GGGGS |
| 7GS linker | 137 | SGGSGGS |
| 8GS linker | 138 | GGGGSGGS |
| 9GS linker | 87 | GGGGSGGGS |
| 10GS linker | 139 | GGGGSGGGGS |
| 15GS linker | 140 | GGGGSGGGGSGGGGS |
| 18GS linker | 141 | GGGGSGGGGSGGGGSGGS |
| 20GS linker | 142 | GGGGSGGGGSGGGGSGGGGS |
| 25GS linker | 143 | GGGGSGGGGSGGGGSGGGGS |
| | | GGGGS |
| 30GS linker | 144 | GGGGSGGGGSGGGGSGGGGS |
| | | GGGGSGGGGS |
| 35GS linker | 145 | GGGGSGGGGSGGGGSGGGGS |
| | | GGGGSGGGGSGGGGS |
| 40GS linker | 146 | GGGGSGGGGSGGGGSGGGGS |
| | | GGGGSGGGGSGGGGSGGGGS |
| G1 hinge | 147 | EPKSCDKTHTCPPCP |
| 9GS-G1 hinge | 148 | GGGGSGGGSEPKSCDKTHTC |
| | | PPCP |
| Llama upper long | 149 | EPKTPKPQPAAA |
| hinge region | | |
| G3 hinge | 150 | ELKTPLGDTTHTCPRCPEPK |
| | | SCDTPPPCPRCPEPKSCDTP |
| | | PPCPRCPEPKSCDTPPPCPRCP |

Therefore, in an embodiment, the polypeptide comprises a first ISVD capable of specifically binding to the constant domain of human and/or non-human primate TCR according to the present technology and a second ISVD capable of specifically binding to a cell-surface specific target antigen, which are linked by 5GS and/or 9GS linkers.

In another embodiment of the present technology, the polypeptide comprises a first ISVD capable of specifically binding to the constant domain of human and/or non-human primate TCR according to the present technology and a second ISVD capable of specifically binding to a cell-surface specific target antigen, which are linked by 9GS linkers.

The inventors surprisingly found that such a configuration can increase the efficiency of the polypeptide in eliciting a T cell-mediated cytotoxic response.

It will be appreciated (as is also demonstrated in the Examples section) that the ISVD binding TCR and the ISVDs binding the first antigen on a target cell can be positioned in any order in the multispecific-multivalent polypeptide of the present technology.

Accordingly, it is an embodiment of the present technology that the polypeptide comprises or consists of the following, in the order starting from the N-terminus of the polypeptide: a first ISVD specifically binding to TCR, a second ISVD specifically binding to a cell-surface specific target antigen, and an optional binding unit providing the polypeptide with increased half-life as defined herein. In one embodiment, the ISVDs are linked by a 9GS linker. The binding unit providing the polypeptide with increased half-life is preferably an ISVD, that preferably binds to serum albumin.

Such configurations of the polypeptide can provide for strong potencies with regards to treating cancer.

Once again, it is not excluded that other binding units/building blocks such as additional ISVDs binding to additional antigens on a target cell, or binding to another target, may be present in the polypeptide. Moreover, the possibility is not excluded that other binding units/building blocks such as ISVDs can be placed in between. For instance, as described further below (see in particular, section 5.4 “(In vivo) half-life extension” below), the polypeptide can further comprise another ISVD specifically binding to human serum albumin that can even be located between e.g. the “first ISVD” and “second ISVD”.

The second ISVD of the polypeptide of the present technology specifically binds to an antigen on a target cell, preferably a cancer cell. A “target cell” as referred to herein, is a cell that presents a particular antigen on its surface. In one embodiment, the “target cell” is a cancer cell.

The membrane (also called plasma membrane or phospholipid bilayer) surrounds the cytoplasm of a cell, which is the outer boundary of the cell, i.e. the membrane is the surface of the cell. This membrane serves to separate and protect a cell from its surrounding environment and is made mostly from a double layer of phospholipids. Embedded within this membrane is a variety of protein molecules, such as channels, pumps and cellular receptors. Since the membrane is fluid, the protein molecules can travel within the membrane. The term “antigen on a target cell” as used herein denotes a molecule, which is displayed on the surface of a cell. In most cases, this molecule will be located in or on the plasma membrane of the cell such that at least part of this molecule remains accessible from outside the cell in tertiary form. A non-limiting example of a cell surface molecule, which is located in the plasma membrane, is a transmembrane protein comprising, in its tertiary conformation, regions of hydrophilicity and hydrophobicity. Here, at least one hydrophobic region allows the cell surface molecule to be embedded or inserted in the hydrophobic plasma membrane of the cell while the hydrophilic regions extend on either side of the plasma membrane into the cytoplasm and extracellular space, respectively.

Said antigen can be any target on a cell, e.g. a tumor antigen. In one embodiment, said antigen is specific for said target cell, e.g. cancer cell, such as a tumor antigen or a tumor associated antigen (TAA) on said cancer cell.

The term “tumor antigen” as used herein may be understood as those antigens that are presented on tumor cells. These antigens can be presented on the cell surface with an extracellular part, which is often combined with a transmembrane and cytoplasmic part of the molecule. These antigens can sometimes be presented only by tumor cells and never by a normal or healthy cell. Tumor antigens can be exclusively expressed on tumor cells or might represent a tumor specific mutation compared to normal cells. In this case, they are called tumor-specific antigens. However, this will not be the case generally. More common are antigens that are presented by tumor cells and normal cells, and they are called “tumor-associated antigens (TAA)”. These tumor-associated antigens can be overexpressed on tumor cells compared to normal cells or are better accessible for antibody binding in tumor cells due to the less compact structure of the tumor tissue compared to normal tissue. TAA are preferably antigens that are expressed on cells of particular tumors, but that are preferably not expressed in normal cells. Often, TAA are antigens that are normally expressed in cells only at particular points in an organism's development (such as during fetal development) and that are being inappropriately expressed in the organism at the present point of development, or are antigens not expressed in normal tissues or cells of an organ now expressing the antigen.

In an embodiment, said first antigen on a target cell is a tumor associated antigen (TAA).

In an embodiment, said first antigen on a target cell is present more abundantly on a cancer cell than on a normal cell. The antigen on a target cell is preferably a tumor associated antigen (TAA).

In an embodiment, said first antigen on a target cell is a tumor antigen or a tumor specific antigen (TSA).

In one embodiment, the multispecific-multivalent polypeptides of the current technology comprise a second ISVD that specifically binds to CD123 or Glypican-3.

In a further embodiment, the polypeptide according to the present technology further comprises a third ISVD, which specifically binds to a second antigen on a target cell.

The target cell bound by the polypeptides of the present technology relates in particular to mammalian cells, preferably to primate cells, and even more preferably to human cells. The target cell is preferably a hyperproliferative cell such as e.g. a cancer cell.

In another embodiment of the present technology, the multispecific-multivalent polypeptide exhibits reduced binding by pre-existing antibodies in human serum. To this end, in one embodiment of the present technology, the polypeptide exhibits a valine (V) at amino acid position 11 and a leucine (L) at amino acid position 89 (according to Kabat numbering) in at least one ISVD (and preferably the ISVD at the C-terminal end of the polypeptide), but preferably in each ISVD.

In another embodiment of the present technology, the polypeptide exhibits an extension of 1 to 5 (preferably naturally occurring) amino acids, such as a single alanine (A) extension, at the C-terminus of the C-terminal ISVD. The C-terminus of an ISVD is normally VTVSS (SEQ ID NO: 88).

In another embodiment of the present technology, the polypeptide exhibits a lysine (K) or glutamine (Q) at position 110 (according to Kabat numbering) in at least one ISVD.

In another embodiment of the present technology, the ISVD exhibits a lysine (K) or glutamine (Q) at position 112 (according to Kabat numbering) in at least on ISVD. In these embodiments, the C-terminus of the ISVD is VKVSS (SEQ ID NO: 89), VQVSS (SEQ ID NO: 90), VTVKS (SEQ ID NO: 91), VTVQS (SEQ ID NO: 92), VKVKS (SEQ ID NO: 93), VKVQS (SEQ ID NO: 94), VQVKS (SEQ ID NO: 95), or VQVQS (SEQ ID NO: 96) such that after addition of a single alanine the C-terminus of the polypeptide for example exhibits the sequence VTVSSA (SEQ ID NO: 97), VKVSSA (SEQ ID NO: 98), VQVSSA (SEQ ID NO: 99), VTVKSA (SEQ ID NO: 100), VTVQSA (SEQ ID NO: 101), VKVKSA (SEQ ID NO: 102), VKVQSA (SEQ ID NO: 103), VQVKSA (SEQ ID NO: 104), or VQVQSA (SEQ ID NO: 105), preferably VTVSSA.

In another embodiment of the present technology, the polypeptide exhibits a valine (V) at amino acid position 11 and a leucine (L) at amino acid position 89 (according to Kabat numbering) in at least the C-terminal ISVD, optionally a lysine (K) or glutamine (Q) at position 110 (according to Kabat numbering) in at least one ISVD, and exhibits an extension of 1 to 5 (preferably naturally occurring) amino acids, such as a single alanine (A) extension, at the C-terminus of the C-terminal ISVD (such that the C-terminus of the polypeptide for example consists of the sequence VTVSSA, VKVSSA or VQVSSA, preferably VTVSSA). See e.g. WO2012/175741 and WO2015/173325 for further information in this regard.

As will be clear from the further description above and herein, the ISVDs of the present technology can be used as “building blocks” to form polypeptides of the present technology, e.g., by suitably combining them with other groups, residues, moieties or binding units, in order to form compounds or constructs as described herein (such as, without limitations, the bi-/tri-/tetra-/multivalent and bi-/tri-/tetra-/multispecific polypeptides of the present technology described herein), which combine within one molecule one or more desired properties or biological functions. A polypeptide with multiple ISVDs is also referred to herein as a “construct” or “ISVD format”.

### 5.3 Specificity

The terms “specificity”, “binding specifically” or “specific binding” refer to the number of different target molecules, such as antigens, from the same organism to which a particular binding unit, such as an ISVD, can bind with sufficiently high affinity (see below). “Specificity”, “binding specifically” or “specific binding” are used interchangeably herein with “selectivity”, “binding selectively” or “selective binding”. Binding units, such as ISVDs, preferably specifically bind to their designated targets.

The specificity/selectivity of a binding unit can be determined based on affinity. The affinity denotes the strength or stability of a molecular interaction. The affinity is commonly given as by the K_{D}, or dissociation constant, which is expressed in units of mol/liter (or M). The affinity can also be expressed as an association constant, K_{A}, which equals 1/K_{D} and is expressed in units of (mol/liter)⁻¹ (or M⁻¹).

The affinity is a measure for the binding strength between a moiety and a binding site on the target molecule: the lower the value of the K_{D}, the stronger the binding strength between a target molecule and a targeting moiety.

Typically, binding units used in the present technology (such as ISVDs) will bind to their targets with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10¹ to 10¹² liter/moles or more and more preferably 10⁸ to 10¹² liter/moles).

Any K_{D} value greater than 10⁻⁴ mol/liter (or any K_{A} value lower than 10⁴ liters/mol) is generally considered to indicate non-specific binding.

The K_{D} for biological interactions, such as the binding of immunoglobulin sequences to an antigen, which are considered specific are typically in the range of 10⁻¹ moles/liter (10000 nM or 10 μM) to 10⁻¹² moles/liter (0.001 nM or 1 μM) or less.

Accordingly, specific/selective binding may mean that—using the same measurement method, e.g. SPR—a binding unit (or polypeptide comprising the same) binds to TCR with a K_{D} value of 10⁻⁵ to 10¹² moles/liter or less and binds to related targets with a K_{D} value greater than 10⁻⁴ moles/liter.

Thus, the ISVD preferably exhibits at least half the binding affinity, more preferably at least the same binding affinity, to human TCR as compared to an ISVD consisting of the amino acid of SEQ ID NO: 1, wherein the binding affinity is measured using the same method, such as SPR.

Specific binding to a certain target from a certain species does not exclude that the binding unit can also specifically bind to the analogous target from a different species. For example, specific binding to human TCR does not exclude that the binding unit (or a polypeptide comprising the same) can also specifically bind to TCR from cynomolgus monkeys.

When an ISVD is said to exhibit “improved cross-reactivity for binding to human and non-human primate TCR” compared to another ISVD, it means that for said ISVD the ratio of the binding activity (such as expressed in terms of K_{D} or k_{off}) for human TCR and for non-human primate TCR is lower than that same ratio calculated for the other ISVD in the same assay.

Good cross-reactivity for binding to human and non-human primate TCR allows for the assessment of toxicity of a multispecific T cell engaging polypeptide in preclinical studies conducted in non-human primates.

Specific binding of a binding unit to its designated target can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as the other techniques mentioned herein.

The dissociation constant may be the actual or apparent dissociation constant, as will be clear to the skilled person. Methods for determining the dissociation constant will be clear to the skilled person, and for example include the techniques mentioned below. In this respect, it will also be clear that it may not be possible to measure dissociation constants of more than 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].

The affinity of a molecular interaction between two molecules can be measured via different techniques known per se, such as the well-known surface plasmon resonance (SPR) biosensor technique (see for example Ober et al. 2001, Intern. Immunology 13: 1551-1559). The term “surface plasmon resonance”, as used herein, refers to an optical phenomenon that allows for the analysis of real-time biospecific interactions by detection of alterations in protein concentrations within a biosensor matrix, where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIAcore® system (BIAcore International AB, a GE Healthcare company, Uppsala, Sweden and Piscataway, NJ). For further descriptions, see Jonsson et al. (1993, Ann. Biol. Clin. 51: 19-26), Jonsson et al. (1991, Biotechniques 11: 620-627), Johnsson et al. (1995, J. Mol. Recognit. 8: 125-131), and Johnnson et al. (1991, Anal. Biochem. 198: 268-277).

Another well-known biosensor technique to determine affinities of biomolecular interactions is bio-layer interferometry (BLI) (see for example Abdiche et al. 2008, Anal. Biochem. 377: 209-217). The term “bio-layer Interferometry” or “BLI”, as used herein, refers to a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BLI can for example be performed using the well-known Octet® Systems (ForteBio, a division of Pall Life Sciences, Menlo Park, USA).

Alternatively, affinities can be measured in Kinetic Exclusion Assay (KinExA) (see for example Drake et al. 2004, Anal. Biochem., 328: 35-43), using the KinExA® platform (Sapidyne Instruments Inc, Boise, USA). The term “KinExA”, as used herein, refers to a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

The GYROLAB® immunoassay system provides a platform for automated bioanalysis and rapid sample turnaround (Fraley et al. 2013, Bioanalysis 5: 1765-74).

In one embodiment, the ISVD of the present technology has an on-rate constant (kₒₙ) for binding to the human TCR selected from the group consisting of at least about 10³ M⁻¹s⁻¹, at least about 10⁴ M⁻¹s⁻¹, and at least about 10¹ M⁻¹s⁻¹, preferably as measured by SPR, preferably performed on a ProteOn XPR36 instrument, preferably at 25°.

In one embodiment, the ISVD of the present technology has an a kₒₙ for binding to the non-human primate TCR selected from the group consisting of at least about 10³ M⁻¹s⁻¹, at least about 10⁴ M⁻¹s⁻¹, and at least about 10⁵ M⁻¹s⁻¹, preferably as measured by SPR, preferably performed on a ProteOn XPR36 instrument, preferably at 25° C.

In one embodiment, the ISVD of the present technology has a k_{off} for binding to the human TCR selected from the group consisting of at most about 10⁻¹ s⁻¹, at most about 10⁻² s⁻¹, at most about 10⁻³ s⁻¹, and at most about 10⁻⁴ s⁻¹, preferably as measured by SPR, preferably performed on a ProteOn XPR36 instrument, preferably at 25° C.

In one embodiment, the ISVD of the present technology has a k_{off} for binding to the non-human primate TCR selected from the group consisting of at most about 10⁻¹ s⁻¹, at most about 10⁻² s⁻¹, at most about 10⁻³ s⁻¹, and at most about 10⁻⁴ s⁻¹, preferably as measured by SPR, preferably performed on a ProteOn XPR36 instrument, preferably at 25° C.

ISVDs with a Y, F, H, K, L or R at position 99 showed a particularly advantageous k_{off} compared to ISVDs with a different amino acid at position 99. As such, in one embodiment, the ISVD has Y, F, H, K, L or R at position 99. In one embodiment the ISVD has Y, F, H, or R at position 99.

ISVDs with a W, A, E, F, H, I, K, L, Q, R, S, T, V or Y at position 10³ (Kabat numbering) showed a particular advantageous k_{off} compared to ISVDs with another amino acid at position 10³. As such, in one embodiment, the ISVD has W, A, E, F, H, I, K, L, Q, R, S, T, V or Y at position 10³. In one embodiment the ISVD has W, A, E, F, H, I, K, L, Q, S, T or V at position 10³.

ISVDs with an A, E, F, H, I, K, L, N, P, Q, R, S, T, V or Y at position 61 (Kabat numbering) showed a k_{off} that was as good as a reference TCE ISVD with a D at position 61. Since isomerization was observed at D61 in said reference ISVD, there was a need to obtain ISVDs with an at least similar off-rate with a different amino acid in position 61. Therefore, in one embodiment the ISVD has A, E, F, H, I, K, L, N, P, Q, R, S, T, V or Y at position 61, preferably the ISVD has an E at position 61.

In one embodiment, the ISVD of the present technology has an affinity (K_{D}) for binding to the human TCR selected from the group consisting of at most about 10⁻⁶ M, at most about 10⁻⁷ M, at most about 10⁻⁸ M, and at most about 10⁻⁹ M, preferably as measured by SPR, preferably performed on a ProteOn XPR36 instrument, preferably at 25° C.

In one embodiment, the ISVD of the present technology has a K_{D} for binding to the non-human primate TCR selected from the group consisting of at most about 10⁻⁵ M, at most about 10⁻⁶ M, at most about 10⁻⁷ M, and at most about 10⁻⁸ M, preferably as measured by SPR, preferably performed on a ProteOn XPR36 instrument, preferably at 25° C.

In some embodiments, the TCR binding ISVDs of the present technology bind to the human TCR with the same or lower off-rate constant (k_{off}) compared to SEQ ID NO: 1. In some embodiments, the ISVD of the present technology binds to non-human primate TCR with the same or lower k_{off} compared to an ISVD of SEQ ID NO: 1. In some embodiments, the TCR binding ISVD of the present technology bind to the human TCR with the same or lower off-rate constant (k_{off}) compared to ISVD T0170056G05 (disclosed as SEQ ID NO: 50 in WO2016180969).

In some embodiments, the ISVD of the present technology binds to non-human primate TCR with the same or lower k_{off} compared to ISVD T0170056G05 (disclosed as SEQ ID NO: 50 in WO2016180969). The off-rate (k_{off}) can be measured by any method known to the skilled person. In one embodiment, the off-rate (k_{off}) is measured by surface plasmon resonance (SPR), preferably performed on a ProteOn XPR36 instrument, preferably at 25° C.

The inventors found that ISVDs according to the present technology with an affinity (K_{D}) that was the same or higher than reference ISVD T017000700 (SEQ ID NO: 1) also had a similar or higher potency than said reference ISVD when tested in cell killing assays.

Surprisingly, the inventors found a subset of the ISVDs of the present technology with an affinity (K_{D}) for binding to the human TCR that was more than 40-fold lower than that of reference ISVD T017000700 (SEQ ID NO: 1), but that maintained high potency in cell killing assays. These particular ISVDs with an E in position 61 and a Q, S or T in position 99 (Kabat numbering) present an interesting opportunity in the generation of biotherapeutics with a better biodistribution.

In one embodiment, the ISVD of the present technology has improved cross-reactivity for binding to human and non-human primate TCR compared to ISVD T0170056G05 (disclosed as SEQ ID NO: 50 in WO2016180969). Accordingly, in a particular embodiment, the ISVD of the present technology, has a k_{off} for binding to non-human primate TCR which is within 5-fold range of the k_{off} for binding to human TCR.

In another embodiment, the ISVD of the present technology binds to human TCR with the same or lower K_{D} compared to SEQ ID NO: 1, preferably as measured by surface plasmon resonance (SPR) preferably performed on a ProteOn XPR36 instrument, preferably at 25° C. In another embodiment, the ISVD of the present technology binds to non-human primate TCR with the same or lower K_{D} compared to SEQ ID NO: 1, preferably as measured by surface plasmon resonance (SPR) preferably performed on a ProteOn XPR36 instrument, preferably at 25° C. In another embodiment, the ISVD of the present technology binds to human TCR with the same or lower K_{D} compared to ISVD T0170056G05 (disclosed as SEQ ID NO: 50 in WO2016180969), preferably as measured by surface plasmon resonance (SPR) preferably performed on a ProteOn XPR36 instrument, preferably at 25° C. In another embodiment, the ISVD of the present technology binds to non-human primate TCR with the same or lower K_{D} compared to ISVD T0170056G05 (disclosed as SEQ ID NO: 50 in WO2016180969), preferably as measured by surface plasmon resonance (SPR), preferably performed on a ProteOn XPR36 instrument, preferably at 25° C.

In one embodiment, the ISVD of the present technology has (i) an affinity (K_{D}) for binding to the human TCR selected from the group consisting of at most about 10⁻⁶ M, at most about 10⁻⁷ M, at most about 10⁻⁸ M, at most about 10⁻⁸ M, and at most about 10⁻⁹ M and (ii) has a K_{D} for binding to the non-human primate TCR selected from the group consisting of at most about 10⁻⁵ M, at most about 10⁻⁶ M, at most about 10⁻⁷ M, and at most about 10⁻⁸ M, preferably as measured by SPR, preferably performed on a ProteOn XPR36 instrument, preferably at 25° C.

In one embodiment, the ISVD of the present technology shows improved cross-reactivity, i.e., the ratio of binding activity (such as expressed in terms of K_{D} or K_{off}) for binding to human and non-human primate TCR is lower as compared to that same ratio for ISVD T0170056G05 (disclosed as SEQ ID NO: 50 in WO2016180969), said K_{D} or K_{off} preferably determined by SPR, preferably performed on a ProteOn XPR36 instrument, preferably at 25° C.

For instance, the ISVD of the present technology shows a lower difference in human-cynomolgus cross-reactivity based on K_{D} compared to the difference in human-cynomolgus cross-reactivity for ISVD T0170056G05 (disclosed as SEQ ID NO: 50 in WO2016180969). Preferably, the K_{D} is determined by SPR, preferably performed on a ProteOn XPR36 instrument, preferably at 25° C.

### 5.4 (In Vivo) Half-Life Extension

The polypeptide according to the present technology may, as disclosed here above, further comprise one or more other groups, residues, moieties or binding units, optionally linked via one or more peptidic linkers, in which said one or more other groups, residues, moieties or binding units provide the polypeptide with increased (in vivo) half-life, compared to the corresponding polypeptide without said one or more other groups, residues, moieties or binding units. In vivo half-life extension means, for example, that the polypeptide exhibits an increased half-life in a mammal, such as a human subject, after administration. Half-life can be expressed for example as t½beta.

The type of groups, residues, moieties or binding units is not generally restricted and may for example be selected from the group consisting of a polyethylene glycol molecule, serum proteins or fragments thereof, binding units that can bind to serum proteins, an Fc portion, and small proteins or peptides that can bind to serum proteins.

More specifically, said one or more other groups, residues, moieties or binding units that provide the polypeptide with increased half-life can be selected from the group consisting of binding units that can bind to serum albumin, such as human serum albumin, or a serum immunoglobulin, such as IgG, and preferably is a binding unit that can bind to human serum albumin. The binding unit is preferably an ISVD.

For example, WO 04/041865 describes ISVDs binding to serum albumin (and in particular human serum albumin) that can be linked to other proteins (such as one or more other ISVDs binding to a desired target) in order to increase the half-life of said protein.

The international application WO 06/122787 describes a number of ISVDs against (human) serum albumin. These ISVDs include the ISVD called Alb-1 (SEQ ID NO: 52 in WO 06/122787) and humanized variants thereof, such as Alb-8 (SEQ ID NO: 62 in WO 06/122787). Again, these can be used to extend the half-life of therapeutic proteins and polypeptide and other therapeutic entities or moieties.

Moreover, WO2012/175400 describes a further improved version of Alb-1, called Alb-23.

In an embodiment of the present technology, the polypeptide comprises a serum albumin binding moiety selected from Alb-1, Alb-3, Alb-4, Alb-5, Alb-6, Alb-7, Alb-8, Alb-9, Alb-10 and Alb-23, preferably Alb-8 or Alb-23 or its variants, as shown in pages 7-9 of WO2012/175400 and the albumin binders described in WO 2012/175741, WO2015/173325, WO2017/080850, WO2017/085172, WO2018/104444, WO2018/134235, WO2018/134234.

In another embodiment, the polypeptide comprises a serum albumin binding moiety selected from Table A-3.

TABLE A-3
Serum albumin binding ISVD sequences
(“ID” refers to the SEQ ID NO as used herein)

| Name | ID | Amino acid sequence |
|---|---|---|
| Alb8 | 118 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVTVSS |
| Alb8-A | 119 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVTVSSA |
| Alb23 | 120 | EVQLLESGGGLVQPGGSLRLSCAASGFTFRSFGMSWVRQAPGKGPEWVSSISG |
| | | SGSDTLYADSVKGRFTISRDNSKNTLYLQMNSLRPEDTAVYYCTIGGSLSRSSQG |
| | | TLVTVSS |
| Alb23-A | 121 | EVQLLESGGGLVQPGGSLRLSCAASGFTFRSFGMSWVRQAPGKGPEWVSSISG |
| | | SGSDTLYADSVKGRFTISRDNSKNTLYLQMNSLRPEDTAVYYCTIGGSLSRSSQG |
| | | TLVTVSSA |
| Alb83 | 122 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTATYYCTIGGSLSRSSQGT |
| | | LVTVSS |
| Alb83-A | 123 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTATYYCTIGGSLSRSSQGT |
| | | LVTVSSA |
| Alb132 | 124 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTATYYCTIGGSLSRSSQ |
| | | GTLVTVSS |
| Alb132-A | 125 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTATYYCTIGGSLSRSSQ |
| | | GTLVTVSSA |
| Alb73 | 126 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVKVSS |
| Alb73-A | 127 | EVQLVESGGGLVQPGNSLRLSCAASGFTFSSFGMSWVRQAPGKGLEWVSSISG |
| | | SGSDTLYADSVKGRFTISRDNAKTTLYLQMNSLRPEDTAVYYCTIGGSLSRSSQGT |
| | | LVKVSSA |
| Alb82 | 128 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVTVSS |
| Alb82-A | 129 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVTVSSA |
| Alb199 | 130 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVKVSS |
| Alb199-A | 131 | EVQLVESGGGVVQPGNSLRLSCAASGFTFSSFGMSWVRQAPGKGLEWVSSISG |
| | | SGSDTLYADSVKGRFTISRDNAKTTLYLQMNSLRPEDTALYYCTIGGSLSRSSQGT |
| | | LVKVSSA |
| Alb23002 | 132 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVTVSS |
| Alb223 | 133 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVTVSSA |
| Alb216 | 134 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVKVSS |
| Alb216-A | 135 | EVQLVESGGGVVQPGGSLRLSCAASGFTFRSFGMSWVRQAPGKGPEWVSSIS |
| | | GSGSDTLYADSVKGRFTISRDNSKNTLYLQMNSLRPEDTALYYCTIGGSLSRSSQ |
| | | GTLVKVSSA |

When such an ISVD binding to human serum albumin is at a C-terminal position it can exhibit a C-terminal alanine (A) or glycine (G) extension (preferably A). In some embodiments of the present technology, the ISVD binding to human serum albumin is at another position than the C-terminal position (i.e. is not the C-terminal ISVD of the polypeptide).

### 5.5 Nucleic Acid Molecules

Also provided is a nucleic acid molecule encoding the ISVDs or polypeptides as disclosed herein.

A “nucleic acid molecule” (used interchangeably with “nucleic acid”) is a chain of nucleotide monomers linked to each other via a phosphate backbone to form a nucleotide sequence. A nucleic acid may be used to transform/transfect a host cell or host organism, e.g. for expression and/or production of a polypeptide. Suitable hosts or host cells for production purposes will be clear to the skilled person, and may for example be any suitable fungal, prokaryotic or eukaryotic cell or cell line or any suitable fungal, prokaryotic or eukaryotic organism. A (non-human) host or host cell comprising a nucleic acid encoding the polypeptide is also encompassed by the technology.

A nucleic acid may be for example DNA, RNA, or a hybrid thereof, and may also comprise (e.g. chemically) modified nucleotides, like PNA. It can be single- or double-stranded and is preferably in the form of double-stranded DNA. For example, the nucleotide sequences may be genomic DNA or cDNA.

The nucleic acids can be prepared or obtained in a manner known per se, and/or can be isolated from a suitable natural source. Nucleotide sequences encoding naturally occurring (poly)peptides can for example be subjected to site-directed mutagenesis, so as to provide a nucleic acid molecule encoding polypeptide with sequence variation. Also, as will be clear to the skilled person, to prepare a nucleic acid, also several nucleotide sequences, such as at least one nucleotide sequence encoding a targeting moiety and for example nucleic acids encoding one or more linkers can be linked together in a suitable manner.

Techniques for generating nucleic acids will be clear to the skilled person and may for instance include, but are not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers.

### 5.6 Vectors

Also provided is a vector comprising the nucleic acid molecule encoding the ISVDs or polypeptides as disclosed herein.

A “vector” as used herein is a vehicle suitable for carrying genetic material into a cell. A vector includes naked nucleic acids, such as plasmids or mRNAs, or nucleic acids embedded into a bigger structure, such as liposomes or viral vectors.

Vectors generally comprise at least one nucleic acid that is optionally linked to one or more regulatory elements, such as for example one or more suitable promoter(s), enhancer(s), terminator(s), etc.). The vector preferably is an expression vector, i.e. a vector suitable for expressing an encoded polypeptide or construct under suitable conditions, e.g. when the vector is introduced into a (e.g. human) cell. For DNA-based vectors, this usually includes the presence of elements for transcription (e.g. a promoter and a polyA signal) and translation (e.g. Kozak sequence).

Preferably, in the vector, said at least one nucleic acid and said regulatory elements are “operably linked” to each other, by which is generally meant that they are in a functional relationship with each other. For instance, a promoter is considered “operably linked” to a coding sequence if said promoter is able to initiate or otherwise control/regulate the transcription and/or the expression of a coding sequence (in which said coding sequence should be understood as being “under the control of” said promotor). Generally, when two nucleotide sequences are operably linked, they will be in the same orientation and usually also in the same reading frame. They will usually also be essentially contiguous, although this may also not be required.

Preferably, any regulatory elements of the vector are such that they are capable of providing their intended biological function in the intended host cell or host organism.

For instance, a promoter, enhancer or terminator should be “operable” in the intended host cell or host organism, by which is meant that for example said promoter should be capable of initiating or otherwise controlling/regulating the transcription and/or the expression of a nucleotide sequence—e.g. a coding sequence—to which it is operably linked.

### 5.7 Compositions

The technology also provides a composition comprising at least one ISVD or polypeptide as disclosed herein, at least one nucleic acid molecule encoding an ISVD or polypeptide as disclosed herein or at least one vector comprising such a nucleic acid molecule. The composition may be a pharmaceutical composition. The composition may further comprise at least one pharmaceutically acceptable carrier, diluent or excipient and/or adjuvant, and optionally comprise one or more further pharmaceutically active polypeptides and/or compounds.

### 5.8 Host Organisms

The technology also pertains to host cells or host organisms comprising the ISVDs or polypeptides as disclosed herein, the nucleic acid encoding the ISVDs or polypeptides as disclosed herein, and/or the vector comprising the nucleic acid molecule encoding the ISVDs or polypeptides as disclosed herein.

Suitable host cells or host organisms are clear to the skilled person, and are for example any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

### 5.9 Methods and Uses of the ISVDs and Polypeptides

The technology also provides a method for producing the ISVDs or polypeptides as disclosed herein. The method may comprise transforming/transfecting a host cell or host organism with a nucleic acid encoding the ISVD or polypeptide, expressing the ISVD or polypeptide in the host, optionally followed by one or more isolation and/or purification steps. Specifically, the method may comprise:
a) expressing, in a suitable host cell or (non-human) host organism or in another suitable expression system, a nucleic acid sequence encoding the ISVD or polypeptide according to the present technology; optionally followed by:
b) isolating and/or purifying the polypeptide.

Suitable host cells or host organisms for production purposes will be clear to the skilled person, and may for example be any suitable fungal, prokaryotic or eukaryotic cell or cell line or any suitable fungal, prokaryotic or eukaryotic organism. Specific examples include HEK293 cells, CHO cells, Escherichia coli or Pichia pastoris. The most preferred host is Pichia pastoris.

Typically, the multispecific-multivalent polypeptides of the current technology combine high affinity antigen recognition on the target cell with T cell activation, resulting in an activation that is independent of the T cells' natural specificity. The first ISVD of the polypeptide of the present technology has high affinity for/specifically binds to an effector cell, preferably the TCR of said effector cell, and even more preferably the constant domain of the TCR.

An effector cell is a cell comprising a TCR complex, preferably an immune cell, such as a T cell, preferably a CD4+T-helper cell (also known as CD4 cell, T-helper cell or T4 cell), more preferably a Cytotoxic T cell (also known as TC cell, CTL or CD8+ T cells) or Natural Killer T cells (NKT cells). In some embodiments, the cell is present in vivo. In some embodiments, the cell is present in vitro. The effector cell of the present technology relates in particular to mammalian cells, preferably to primate cells, and even more preferably to human cells.

“T cell activation” as used herein refers to one or more cellular response(s) of a T cell, e.g. a cytotoxic T cell, such as selected from: proliferation, differentiation, cytokine secretion, cytotoxic effector molecule release, cytotoxic activity, expression of activation markers, and redirected target cell lysis. The multispecific-multivalent polypeptides of the current technology are capable of inducing T cell activation. Suitable assays to measure T cell activation are known in the art described herein, for instance as described in WO 99/54440 or by Schlereth et al. 2005 (Cancer Immunol. Immunother. 20: 1-12), or as exemplified in the examples or below.

In an embodiment, the present technology relates to a multispecifc-multivalent polypeptide as described herein, wherein said polypeptide induces T cell activation. Preferably, the polypeptide of the present technology induces T cell activation only when said second and/or further ISVD is bound to an antigen on a target cell.

In an embodiment, the present technology relates to a multispecifc-multivalent polypeptide as described herein, wherein said T cell activation depends on presenting said polypeptide bound to said first antigen on a target cell to a T cell.

T cell activation by the polypeptides of the present technology can be monitored by upregulation of CD69, CD25 and various cell adhesion molecules, de novo expression and/or release of cytokines (e.g., IFN-γ, TNF-α, IL-6, IL-2, IL-4 and IL-10), upregulation of granzyme and perforin expression, and/or cell proliferation, membrane blebbing, activation of procaspases 3 and/or 7, fragmentation of nuclear DNA and/or cleavage of caspase substrate poly (ADPribose) polymerase. Preferably, redirected lysis of target cells by multispecific-multivalent polypeptides is independent of T cell receptor specificity, presence of MHC class I and/or 32 microglobulin, and/or of any co-stimulatory stimuli.

In an embodiment, the present technology relates to a multispecific-multivalent polypeptide as described herein, wherein said T cell activation is independent from MHC recognition.

The multispecific-multivalent polypeptides of the present technology show redirected lysis in vitro with previously unstimulated peripheral polyclonal CD8⁺- and CD4⁺-positive T cells. The redirected lysis of target cells via the recruitment of T cells by the polypeptides of the present technology involves cytolytic synapse formation and delivery of perforin and granzymes. Cell lysis by T cells has been described, e.g. by Atkinson and Bleackley 1995 (Crit. Rev. Immunol 15(3-4): 359-384). Preferably, the engaged T cells are capable of serial target cell lysis, and are not affected by immune escape mechanisms interfering with peptide antigen processing and presentation, or clonal T cell differentiation (see, for example, WO 2007/042261). In vitro, redirected lysis is seen at low picomolar concentrations, suggesting that very low numbers of the polypeptides of the present technology need to be bound to target cells for triggering T cells. Accordingly, the present technology relates to potent polypeptides. Preferably, the multispecific-multivalent polypeptide of the current technology mediates killing of target cells, e.g. cancer cells, such as stimulating T cells in pore formation and delivering pro-apoptotic components of cytotoxic T cell granules.

In an embodiment, the present technology relates to a multispecific-multivalent polypeptide as described herein, wherein said T cell activation causes one or more cellular response of said T cell, wherein said cellular response is selected from the group consisting of proliferation, differentiation, cytokine secretion, cytotoxic effector molecule release, cytotoxic activity, expression of activation markers and redirected target cell lysis.

As used herein, the term “potency” is a measure of the biological activity of an agent, such as a polypeptide or an ISVD. Potency of an agent can be determined by any suitable method known in the art, such as for instance as described in the experimental section. Cell culture-based potency assays are often the preferred format for determining biological activity since they measure the physiological response elicited by the agent and can generate results within a relatively short period of time. Various types of cell based assays, based on the mechanism of action of the product, can be used, including but not limited to proliferation assays, cytotoxicity assays, cell killing assays, reporter gene assays, cell surface receptor binding assays, and assays to measure induction/inhibition of functionally essential proteins or other signal molecules (such as phosphorylated proteins, enzymes, cytokines, cAMP and the like), Ramos B cell depletion model, T cell mediated tumour cell killing assay (for instance as set out in the Examples section), all well known in the art.

In one embodiment, the multispecific-multivalent polypeptides of the present technology showed improved potency and efficacy in both human and cyno T cell mediated killing assays, compared to the same format wherein the first ISVD is replaced by T0170056G05 (disclosed as SEQ ID NO: 50 in WO2016180969).

The “efficacy” (of the polypeptide of the present technology) measures the maximum strength of the effect itself, at saturating polypeptide concentrations. Efficacy indicates the maximum response achievable by the polypeptide of the present technology. It refers to the ability of a polypeptide to produce the desired (therapeutic) effect.

Accordingly, in an embodiment, the present technology relates to a multispecific-multivalent polypeptide as described herein, wherein said T cell activation causes inhibition of an activity of said target cell, such as to delay or minimize the spread of the target cell, to inhibit or delay growth and/or proliferation of the target cell, and/or to kill the target cell (e.g., cause regression of the disorder and/or symptoms), by more than about 10%, such as 20%, 30%, or 40%, or even more than 50%, such as more than 60%, such as 70%, 80% or even more than 90%, such as 100%. In a particular embodiment, the T cell activation causes lysis of the target cell, by more than about 10%, such as 20%, 30%, or 40%, or even more than 50%, such as more than 60%.

In one embodiment, the multispecific-multivalent polypeptide as described herein causes a human T cell to lyse the target cell with an EC50 value selected from the group consisting of at most about 10⁻⁹ M, at most about 10⁻¹⁰ M, and at most about 10⁻¹¹ M, said EC50 value as determined in a T cell mediated killing assay. For instance, the EC50 is determined in a flow cytometry-based assay as set out in Example 8 or 9, or in an impedance-based cytotoxicity assay as set out in Example 10.

In one embodiment, the multispecific-multivalent polypeptides as described herein cause a non-human primate T cell to lyse the target cell with an EC50 value selected from the group consisting of at most about 10⁻⁹ M, and at most about 10⁻¹⁰ M, and at most about 10⁻¹¹ M, said EC50 value as determined in a T cell mediated killing assay. For instance, the EC50 is determined in a flow cytometry-based assay as set out in Example 8 or 9, or in an impedance-based cytotoxicity assay as set out in Example 10.

Multispecific-multivalent polypeptides with a TCR binding ISVD according to the present technology with an E at position 61 and Y or S at position 99 showed particularly high potency in a flow cytometry-based T cell mediated killing assay. As such, in one embodiment, the multispecific-multivalent polypeptide comprises a TCR binding ISVD with E at position 61 and Y or S at position 99. In one embodiment, the multispecific-multivalent polypeptide causes a human T cell to lyse the target cell with an EC50 value of at most about 5·10⁻¹⁰ M, at most about 10⁻¹⁰ M, such as 5·10⁻¹¹ M, said EC50 value as determined in a flow cytometry-based T cell mediated killing assay or impedance-based T cell mediated killing assay. In one embodiment, the multispecific-multivalent polypeptide comprises a TCR binding ISVDs with an E at position 61 and a Y, Q, T or S at position 99 and causes a human T cell to lyse the target cell with an EC50 value of at most about 5·10⁻¹⁰ M, at most about 10⁻¹⁰ M, such as at most 5·10⁻¹¹ M, said EC50 value as determined in a flow cytometry-based T cell mediated killing assay or impedance-based T cell mediated killing assay.

In one embodiment, the multispecific-multivalent polypeptide as described herein, are capable of activating human and/or non-human primate T cells to lyse a target cell with an improved (lower) EC50 value as compared to the same polypeptide wherein the first ISVD is replaced by ISVD T0170056G05 (disclosed as SEQ ID NO: 50 in WO2016180969).

In one embodiment, the multispecific-multivalent polypeptide of the present technology causes a human T cell to lyse the target cell with an improved (lower) EC50 value than that of the same polypeptide wherein the first ISVD is replaced by ISVD T0170056G05 (disclosed as SEQ ID NO: 50 in WO2016180969), said EC50 value as determined in a T cell mediated killing assay. For instance, the EC50 is determined as set out in the examples section.

In one embodiment, the multispecific-multivalent polypeptide of the present technology causes a non-human primate T cell to lyse the target cell with an improved (lower) EC50 value than that of the same polypeptide wherein the first ISVD is replaced by ISVD T0170056G05 (disclosed as SEQ ID NO: 50 in WO2016180969), said EC50 value as determined in a T cell mediated killing assay. For instance, the EC50 is determined as set out in the examples section.

In one embodiment, the multispecific-multivalent polypeptides as described herein, comprising a first ISVD of the present technology, show improved binding to the constant domain of a human and/or of a non-human primate TCR, compared to the same polypeptide wherein the first ISVD is replaced by ISVD T0170056G05 (disclosed as SEQ ID NO: 50 in WO2016180969), i.e. a polypeptide which comprises as first ISVD an ISVD with the CDR sequences of T0170056G05.

The binding characteristics of the ISVDs of the present technology are discussed in more detail below (section 5.3; “specificity”).

In some embodiments, the multispecific-multivalent polypeptides of the present technology have an on-rate constant (kₒₙ) for binding to the human TCR selected from the group consisting of at least about 10³ M⁻¹s⁻¹, at least about 10⁴ M⁻¹s⁻¹, and at least about 10⁵ M⁻¹s⁻¹.

In some embodiments, the multispecific-multivalent polypeptides as described herein have a kₒₙ for binding to the non-human primate TCR selected from the group consisting of at least about 10³ M⁻¹s⁻¹, at least about 10⁴ M⁻¹s⁻¹, at least about 10⁵ M⁻¹s⁻¹, and at least about 10⁶ M⁻¹s⁻¹.

In some embodiments, the multispecific-multivalent polypeptides of the present technology have an off-rate constant (k_{off}) for binding to the human TCR selected from the group consisting of at most about 10⁻¹ s⁻¹, at most about 10⁻² s⁻¹, at most about 10⁻³ s⁻¹, and at most about 10⁻⁴ s⁻¹.

In some embodiments, the multispecific-multivalent polypeptides as described herein have a k_{off} for binding to the non-human primate TCR selected from the group consisting of at most about 10⁻¹ s⁻¹, at most about 10⁻² s⁻¹, at most about 10⁻³ s⁻¹, and at most about 10⁻⁴ s⁻¹.

In some embodiments, the multispecific-multivalent polypeptides of the present technology have an affinity (K_{D}) for binding to the human TCR selected from the group consisting of at most about 10⁻⁵ M, at most about 10⁻⁶ M, at most about 10⁻⁷ M, at most about 10⁻⁸ M, and at most about 10⁻⁹ M.

In some embodiments, the multispecific-multivalent polypeptides of the present technology have a K_{D} for binding to the non-human primate TCR selected from the group consisting of at most about 10⁻⁵ M, most about 10⁻⁶ M, at most about 10⁻⁷ M, and at most about 10⁻⁸ M.

In one embodiment, the kₒₙ, k_{off}, or K_{D} is measured by surface plasmon resonance (SPR). For instance, the kₒₙ, k_{off}, or K_{D} is determined as set out in the Examples section.

In another embodiment, the kₒₙ, k_{off}, or K_{D} is measured by bio-layer interferometry (BLI).

In one embodiment, the multispecific-multivalent polypeptides of the present technology binds to the human TCR with an improved (lower) K_{D} than that of the same polypeptide wherein the first ISVD is replaced by ISVD T0170056G05 (disclosed as SEQ ID NO: 50 in WO2016180969).

In one embodiment, the multispecific-multivalent polypeptides of the present technology binds to the non-human primate TCR with an improved (lower) K_{D} than that of the same polypeptide wherein the first ISVD is replaced by ISVD T0170056G05 (disclosed as SEQ ID NO: 50 in WO2016180969).

The polypeptide, nucleic acid molecule or vector as described, or the composition comprising the ISVD or polypeptide, nucleic acid molecule or vector—preferably the polypeptide or a composition comprising the same—are useful as a medicament.

Accordingly, the technology provides the polypeptide, nucleic acid molecule or vector as described, or a composition comprising the ISVD or polypeptide, nucleic acid molecule or vector for use as a medicament.

Also provided is the polypeptide, nucleic acid molecule or vector as described herein, or a composition comprising the ISVD or polypeptide, nucleic acid molecule or vector for use in the prevention, treatment or amelioration of a disease selected from the group consisting of a proliferative disease, an inflammatory disease, an infectious disease and an autoimmune disease.

Additionally, provided is the polypeptide, nucleic acid molecule or vector as described herein, or a composition comprising the ISVD or polypeptide, nucleic acid molecule or vector for use in the treatment of cancer.

Also provided is a method for the prevention, treatment or amelioration of a disease, wherein said method comprises administering, to a subject in need thereof, a pharmaceutically active amount of the polypeptide, nucleic acid molecule or vector as described herein, or a composition comprising the ISVD or polypeptide, nucleic acid molecule or vector.

Further provided is a method for the prevention, treatment or amelioration of a disease selected from the group consisting of a proliferative disease, an inflammatory disease, an infectious disease and an autoimmune disease, wherein said method comprises administering, to a subject in need thereof, a pharmaceutically active amount of the polypeptide, nucleic acid molecule or vector as described herein, or a composition comprising the ISVD or polypeptide, nucleic acid molecule or vector.

Additionally, provided is a method of treating cancer, wherein said method comprises administering, to a subject in need thereof, a pharmaceutically active amount of the polypeptide, nucleic acid molecule or vector as described herein, or a composition comprising the ISVD or polypeptide, nucleic acid molecule or vector.

Further provided is the use of the polypeptide, nucleic acid molecule or vector as described herein, or a composition comprising the polypeptide, nucleic acid molecule or vector in the preparation of a medicament.

Also provided is the use of the polypeptide, nucleic acid molecule or vector as described herein, or a composition comprising the polypeptide, nucleic acid molecule or vector in the preparation of a medicament for the prevention, treatment or amelioration of a disease selected from the group consisting of a proliferative disease, an inflammatory disease, an infectious disease and an autoimmune disease.

Further provided is the use of the polypeptide, nucleic acid molecule or vector as described herein, or a composition comprising the polypeptide, nucleic acid molecule or vector in the preparation of a pharmaceutical composition, preferably for treating cancer.

A “subject” as referred to in the context of the technology can be any animal, preferably a mammal. Among mammals, a distinction can be made between humans and non-human mammals. Non-human animals may be for example companion animals (e.g. dogs, cats), livestock (e.g. bovine, equine, ovine, caprine, or porcine animals), or animals used generally for research purposes and/or for producing antibodies (e.g. mice, rats, rabbits, cats, dogs, goats, sheep, horses, pigs, non-human primates, such as cynomolgus monkeys, or camelids, such as llama or alpaca).

In the context of prophylactic and/or therapeutic purposes, the subject can be any animal, and more specifically any mammal, but preferably is a human subject.

As used herein, the terms “treat”, “treatment” and “treating” in the context of administering (a) therapy(ies) to a subject refer to the reduction or amelioration of the progression, severity, and/or duration of a disorder associated with a hyperproliferative cell disorder, e.g., cancer, and/or the amelioration of one or more symptoms thereof resulting from the administration of one or more therapies (including, but not limited to, the administration of one or more prophylactic or therapeutic agents). In specific embodiments, the terms “treat”, “treatment” and “treating” in the context of administering a therapy/therapies to a subject refer to the reduction or amelioration of the progression, severity, and/or duration of a hyperproliferative cell disorder, e.g., cancer, refers to a reduction in cancer cells by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control (e.g., a negative control such as phosphate buffered saline). In other embodiments, the terms “treat”, “treatment” and “treating” in the context of administering a therapy, or therapies, to a subject refer to the reduction or amelioration of the progression, severity, and/or duration of a hyperproliferative cell disorder, e.g., cancer, refers to no change in cancer cell number, a reduction in hospitalization time, a reduction in mortality, or an increase in survival time of the subject with cancer.

Substances (including polypeptides, nucleic acid molecules and vectors) or compositions may be administered to a subject by any suitable route of administration, for example by enteral (such as oral or rectal) or parenteral (such as epicutaneous, sublingual, buccal, nasal, intra-articular, intradermal, intramuscular, intraperitoneal, intravenous, subcutaneous, transdermal, or transmucosal) administration. Parenteral administration, such as intramuscular, subcutaneous or intradermal, administration is preferred. Most preferred is subcutaneous administration.

An effective amount of a polypeptide, a nucleic acid molecule or vector as described, or a composition comprising the ISVD or polypeptide, nucleic acid molecule or vector can be administered to a subject in order to provide the intended treatment results.

One or more doses can be administered. If more than one dose is administered, the doses can be administered in suitable intervals in order to maximize the effect of the polypeptide, composition, nucleic acid molecule or vector.

TABLE A-4
Sequences of ISVDs

| | SEQ ID | | | | |
|---|---|---|---|---|---|
| Name | NO | 61 | 99 | 103 | Sequence |
| T017000700 | 1 | D | W | W | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYWGQGT |
| | | | | | LVTVSS |
| T017000937 | 2 | N | W | W | EVQLVESGGGVVQPGGSLRLSCVASGYVHKINFYGWY |
| | | | | | RQAPGKEREKVAHISIGDQTDYANSAKGRFTISRDESK |
| | | | | | NTVYLQMNSLRPEDTAAYYCRALSRIWPYDYWGQGT |
| | | | | | LVTVSS |
| T017000938 | 3 | P | W | W | EVQLVESGGGVVQPGGSLRLSCVASGYVHKINFYGWY |
| | | | | | RQAPGKEREKVAHISIGDQTDYAPSAKGRFTISRDESK |
| | | | | | NTVYLQMNSLRPEDTAAYYCRALSRIWPYDYWGQGT |
| | | | | | LVTVSS |
| T017000939 | 4 | K | W | W | EVQLVESGGGVVQPGGSLRLSCVASGYVHKINFYGWY |
| | | | | | RQAPGKEREKVAHISIGDQTDYAKSAKGRFTISRDESK |
| | | | | | NTVYLQMNSLRPEDTAAYYCRALSRIWPYDYWGQGT |
| | | | | | LVTVSS |
| T017000940 | 5 | R | W | W | EVQLVESGGGVVQPGGSLRLSCVASGYVHKINFYGWY |
| | | | | | RQAPGKEREKVAHISIGDQTDYARSAKGRFTISRDESK |
| | | | | | NTVYLQMNSLRPEDTAAYYCRALSRIWPYDYWGQGT |
| | | | | | LVTVSS |
| T017000941 | 6 | I | W | W | EVQLVESGGGVVQPGGSLRLSCVASGYVHKINFYGWY |
| | | | | | RQAPGKEREKVAHISIGDQTDYAISAKGRFTISRDESKN |
| | | | | | TVYLQMNSLRPEDTAAYYCRALSRIWPYDYWGQGTL |
| | | | | | VTVSS |
| T017000942 | 7 | T | W | W | EVQLVESGGGVVQPGGSLRLSCVASGYVHKINFYGWY |
| | | | | | RQAPGKEREKVAHISIGDQTDYATSAKGRFTISRDESK |
| | | | | | NTVYLQMNSLRPEDTAAYYCRALSRIWPYDYWGQGT |
| | | | | | LVTVSS |
| T017000943 | 8 | H | W | W | EVQLVESGGGVVQPGGSLRLSCVASGYVHKINFYGWY |
| | | | | | RQAPGKEREKVAHISIGDQTDYAHSAKGRFTISRDESK |
| | | | | | NTVYLQMNSLRPEDTAAYYCRALSRIWPYDYWGQGT |
| | | | | | LVTVSS |
| T017000944 | 9 | V | W | W | EVQLVESGGGVVQPGGSLRLSCVASGYVHKINFYGWY |
| | | | | | RQAPGKEREKVAHISIGDQTDYAVSAKGRFTISRDESK |
| | | | | | NTVYLQMNSLRPEDTAAYYCRALSRIWPYDYWGQGT |
| | | | | | LVTVSS |
| T017000945 | 10 | E | W | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIWPYDYWGQGT |
| | | | | | LVTVSS |
| T017000946 | 11 | A | W | W | EVQLVESGGGVVQPGGSLRLSCVASGYVHKINFYGWY |
| | | | | | RQAPGKEREKVAHISIGDQTDYAASAKGRFTISRDESK |
| | | | | | NTVYLQMNSLRPEDTAAYYCRALSRIWPYDYWGQGT |
| | | | | | LVTVSS |
| T017000947 | 12 | Y | W | W | EVQLVESGGGVVQPGGSLRLSCVASGYVHKINFYGWY |
| | | | | | RQAPGKEREKVAHISIGDQTDYAYSAKGRFTISRDESK |
| | | | | | NTVYLQMNSLRPEDTAAYYCRALSRIWPYDYWGQGT |
| | | | | | LVTVSS |
| T017000948 | 13 | L | W | W | EVQLVESGGGVVQPGGSLRLSCVASGYVHKINFYGWY |
| | | | | | RQAPGKEREKVAHISIGDQTDYALSAKGRFTISRDESK |
| | | | | | NTVYLQMNSLRPEDTAAYYCRALSRIWPYDYWGQGT |
| | | | | | LVTVSS |
| T017000949 | 14 | Q | W | W | EVQLVESGGGVVQPGGSLRLSCVASGYVHKINFYGWY |
| | | | | | RQAPGKEREKVAHISIGDQTDYAQSAKGRFTISRDESK |
| | | | | | NTVYLQMNSLRPEDTAAYYCRALSRIWPYDYWGQGT |
| | | | | | LVTVSS |
| T017000950 | 15 | F | W | W | EVQLVESGGGVVQPGGSLRLSCVASGYVHKINFYGWY |
| | | | | | RQAPGKEREKVAHISIGDQTDYAFSAKGRFTISRDESK |
| | | | | | NTVYLQMNSLRPEDTAAYYCRALSRIWPYDYWGQGT |
| | | | | | LVTVSS |
| T017000951 | 16 | S | W | W | EVQLVESGGGVVQPGGSLRLSCVASGYVHKINFYGWY |
| | | | | | RQAPGKEREKVAHISIGDQTDYASSAKGRFTISRDESK |
| | | | | | NTVYLQMNSLRPEDTAAYYCRALSRIWPYDYWGQGT |
| | | | | | LVTVSS |
| T017000952 | 17 | D | W | R | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYRGQGTL |
| | | | | | VTVSS |
| T017000953 | 18 | D | W | A | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYAGQGTL |
| | | | | | VTVSS |
| T017000954 | 19 | D | W | E | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYEGQGTL |
| | | | | | VTVSS |
| T017000955 | 20 | D | W | Y | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYYGQGTL |
| | | | | | VTVSS |
| T017000956 | 21 | D | W | L | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYLGQGTL |
| | | | | | VTVSS |
| T017000957 | 22 | D | W | H | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYHGQGTL |
| | | | | | VTVSS |
| T017000958 | 23 | D | W | - | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYIGQGTL |
| | | | | | VTVSS |
| T017000959 | 24 | D | W | Q | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYQGQGT |
| | | | | | LVTVSS |
| T017000960 | 25 | D | W | V | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYVGQGTL |
| | | | | | VTVSS |
| T017000961 | 26 | D | W | K | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYKGQGTL |
| | | | | | VTVSS |
| T017000962 | 27 | D | W | S | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYSGQGTL |
| | | | | | VTVSS |
| T017000963 | 28 | D | W | G | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYGGQGTL |
| | | | | | VTVSS |
| T017000964 | 29 | D | W | P | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYPGQGTL |
| | | | | | VTVSS |
| T017000965 | 30 | D | W | F | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYFGQGTL |
| | | | | | VTVSS |
| T017000966 | 31 | D | W | T | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIWPYDYTGQGTL |
| | | | | | VTVSS |
| T017000967 | 32 | D | Y | W | EVQLVESGGGVVQPGGSLRLSCVASGYVHKINFYGWY |
| | | | | | RQAPGKEREKVAHISIGDQTDYADSAKGRFTISRDESK |
| | | | | | NTVYLQMNSLRPEDTAAYYCRALSRIYPYDYWGQGTL |
| | | | | | VTVSS |
| T017000974 | 33 | E | Y | W | DVQLVESGGGVVQPGGSLRLSCVASGYVHKINFYGW |
| | | | | | YRQAPGKEREKVAHISIGDQTDYAESAKGRFTISRDES |
| | | | | | KNTVYLQMNSLRPEDTAAYYCRALSRIYPYDYWGQGT |
| | | | | | LVTVSS |
| T017000975 | 34 | D | W | W | DVQLVESGGGVVQPGGSLRLSCVASGYVHKINFYGW |
| | | | | | YRQAPGKEREKVAHISIGDQTDYADSAKGRFTISRDES |
| | | | | | KNTVYLQMNSLRPEDTAAYYCRALSRIWPYDYWGQG |
| | | | | | TLVTVSS |
| T017000976 | 35 | R | Y | W | EVQLVESGGGVVQPGGSLRLSCVASGYVHKINFYGWY |
| | | | | | RQAPGKEREKVAHISIGDQTDYARSAKGRFTISRDESK |
| | | | | | NTVYLQMNSLRPEDTAAYYCRALSRIYPYDYWGQGTL |
| | | | | | VTVSS |
| T017000977 | 36 | V | Y | W | EVQLVESGGGVVQPGGSLRLSCVASGYVHKINFYGWY |
| | | | | | RQAPGKEREKVAHISIGDQTDYAVSAKGRFTISRDESK |
| | | | | | NTVYLQMNSLRPEDTAAYYCRALSRIYPYDYWGQGTL |
| | | | | | VTVSS |
| T017000978 | 37 | E | Y | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIYPYDYWGQGTL |
| | | | | | VTVSS |
| T017000979 | 38 | A | Y | W | EVQLVESGGGVVQPGGSLRLSCVASGYVHKINFYGWY |
| | | | | | RQAPGKEREKVAHISIGDQTDYAASAKGRFTISRDESK |
| | | | | | NTVYLQMNSLRPEDTAAYYCRALSRIYPYDYWGQGTL |
| | | | | | VTVSS |
| T017000980 | 39 | Q | Y | W | EVQLVESGGGVVQPGGSLRLSCVASGYVHKINFYGWY |
| | | | | | RQAPGKEREKVAHISIGDQTDYAQSAKGRFTISRDESK |
| | | | | | NTVYLQMNSLRPEDTAAYYCRALSRIYPYDYWGQGTL |
| | | | | | VTVSS |
| T017000981 | 40 | S | Y | W | EVQLVESGGGVVQPGGSLRLSCVASGYVHKINFYGWY |
| | | | | | RQAPGKEREKVAHISIGDQTDYASSAKGRFTISRDESK |
| | | | | | NTVYLQMNSLRPEDTAAYYCRALSRIYPYDYWGQGTL |
| | | | | | VTVSS |
| T017000982 | 41 | E | Y | Y | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIYPYDYYGQGTLV |
| | | | | | TVSS |
| T017000991 | 42 | E | Y | W | DVQLVESGGGVVQPGGSLRLSCVASGYVHKINFYGW |
| | | | | | YRQAPGKEREKVAHISIGDQTDYAESAKGRFTISRDES |
| | | | | | KNTVYLQMNSLRPEDTAAYYCRALSRIYPYDYWGQGT |
| | | | | | LVTVSS |
| T017000992 | 43 | E | A | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIAPYDYWGQGTL |
| | | | | | VTVSS |
| T017000993 | 44 | E | P | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIPPYDYWGQGTL |
| | | | | | VTVSS |
| T017000994 | 45 | E | D | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIDPYDYWGQGTL |
| | | | | | VTVSS |
| T017000995 | 46 | E | Q | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIQPYDYWGQGT |
| | | | | | LVTVSS |
| T017000996 | 47 | E | E | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIEPYDYWGQGTL |
| | | | | | VTVSS |
| T017000997 | 48 | E | R | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIRPYDYWGQGTL |
| | | | | | VTVSS |
| T017000998 | 49 | E | F | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIFPYDYWGQGTL |
| | | | | | VTVSS |
| T017000999 | 50 | E | S | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRISPYDYWGQGTL |
| | | | | | VTVSS |
| T017001000 | 51 | E | G | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIGPYDYWGQGTL |
| | | | | | VTVSS |
| T017001001 | 52 | E | T | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRITPYDYWGQGTL |
| | | | | | VTVSS |
| T017001002 | 53 | E | H | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIHPYDYWGQGTL |
| | | | | | VTVSS |
| T017001003 | 54 | E | V | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIVPYDYWGQGTL |
| | | | | | VTVSS |
| T017001004 | 55 | E | K | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIKPYDYWGQGTL |
| | | | | | VTVSS |
| T017001005 | 56 | E | L | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRILPYDYWGQGTL |
| | | | | | VTVSS |
| T017001006 | 57 | E | — | W | EVQLVESGGGVVQPGGSLRLSCVASGYVHKINFYGWY |
| | | | | | RQAPGKEREKVAHISIGDQTDYAESAKGRFTISRDESK |
| | | | | | NTVYLQMNSLRPEDTAAYYCRALSRIIPYDYWGQGTL |
| | | | | | VTVSS |

TABLE A-5
Sequences of the CDR region (according to
Kabat definition) of the ISVD variants

| TCE | | | | | | |
|---|---|---|---|---|---|---|
| ISVD | CDR1 | | CDR2 | | CDR3 | |
| SEQ | SEQ | | SEQ | | SEQ | |
| ID | ID | | ID | | ID | |
| NO | NO | Sequence | NO | Sequence | NO | Sequence |
| 1 | 79 | INFYG | 151 | HISIGDQTDYADSAKG | 167 | LSRIWPYDY |
| 2 | 79 | INFYG | 152 | HISIGDQTDYANSAKG | 167 | LSRIWPYDY |
| 3 | 79 | INFYG | 153 | HISIGDQTDYAPSAKG | 167 | LSRIWPYDY |
| 4 | 79 | INFYG | 154 | HISIGDQTDYAKSAKG | 167 | LSRIWPYDY |
| 5 | 79 | INFYG | 155 | HISIGDQTDYARSAKG | 167 | LSRIWPYDY |
| 6 | 79 | INFYG | 156 | HISIGDQTDYAISAKG | 167 | LSRIWPYDY |
| 7 | 79 | INFYG | 157 | HISIGDQTDYATSAKG | 167 | LSRIWPYDY |
| 8 | 79 | INFYG | 158 | HISIGDQTDYAHSAKG | 167 | LSRIWPYDY |
| 9 | 79 | INFYG | 159 | HISIGDQTDYAVSAKG | 167 | LSRIWPYDY |
| 10 | 79 | INFYG | 160 | HISIGDQTDYAESAKG | 167 | LSRIWPYDY |
| 11 | 79 | INFYG | 161 | HISIGDQTDYAASAKG | 167 | LSRIWPYDY |
| 12 | 79 | INFYG | 162 | HISIGDQTDYAYSAKG | 167 | LSRIWPYDY |
| 13 | 79 | INFYG | 163 | HISIGDQTDYALSAKG | 167 | LSRIWPYDY |
| 14 | 79 | INFYG | 164 | HISIGDQTDYAQSAKG | 167 | LSRIWPYDY |
| 15 | 79 | INFYG | 165 | HISIGDQTDYAFSAKG | 167 | LSRIWPYDY |
| 16 | 79 | INFYG | 166 | HISIGDQTDYASSAKG | 167 | LSRIWPYDY |
| 17 | 79 | INFYG | 151 | HISIGDQTDYADSAKG | 167 | LSRIWPYDY |
| 18 | 79 | INFYG | 151 | HISIGDQTDYADSAKG | 167 | LSRIWPYDY |
| 19 | 79 | INFYG | 151 | HISIGDQTDYADSAKG | 167 | LSRIWPYDY |
| 20 | 79 | INFYG | 151 | HISIGDQTDYADSAKG | 167 | LSRIWPYDY |
| 21 | 79 | INFYG | 151 | HISIGDQTDYADSAKG | 167 | LSRIWPYDY |
| 22 | 79 | INFYG | 151 | HISIGDQTDYADSAKG | 167 | LSRIWPYDY |
| 23 | 79 | INFYG | 151 | HISIGDQTDYADSAKG | 167 | LSRIWPYDY |
| 24 | 79 | INFYG | 151 | HISIGDQTDYADSAKG | 167 | LSRIWPYDY |
| 25 | 79 | INFYG | 151 | HISIGDQTDYADSAKG | 167 | LSRIWPYDY |
| 26 | 79 | INFYG | 151 | HISIGDQTDYADSAKG | 167 | LSRIWPYDY |
| 27 | 79 | INFYG | 151 | HISIGDQTDYADSAKG | 167 | LSRIWPYDY |
| 28 | 79 | INFYG | 151 | HISIGDQTDYADSAKG | 167 | LSRIWPYDY |
| 29 | 79 | INFYG | 151 | HISIGDQTDYADSAKG | 167 | LSRIWPYDY |
| 30 | 79 | INFYG | 151 | HISIGDQTDYADSAKG | 167 | LSRIWPYDY |
| 31 | 79 | INFYG | 151 | HISIGDQTDYADSAKG | 167 | LSRIWPYDY |
| 32 | 79 | INFYG | 151 | HISIGDQTDYADSAKG | 168 | LSRIYPYDY |
| 33 | 79 | INFYG | 160 | HISIGDQTDYAESAKG | 168 | LSRIYPYDY |
| 34 | 79 | INFYG | 151 | HISIGDQTDYADSAKG | 167 | LSRIWPYDY |
| 35 | 79 | INFYG | 155 | HISIGDQTDYARSAKG | 168 | LSRIYPYDY |
| 36 | 79 | INFYG | 159 | HISIGDQTDYAVSAKG | 168 | LSRIYPYDY |
| 37 | 79 | INFYG | 160 | HISIGDQTDYAESAKG | 168 | LSRIYPYDY |
| 38 | 79 | INFYG | 161 | HISIGDQTDYAASAKG | 168 | LSRIYPYDY |
| 39 | 79 | INFYG | 164 | HISIGDQTDYAQSAKG | 168 | LSRIYPYDY |
| 40 | 79 | INFYG | 166 | HISIGDQTDYASSAKG | 168 | LSRIYPYDY |
| 41 | 79 | INFYG | 160 | HISIGDQTDYAESAKG | 168 | LSRIYPYDY |
| 42 | 79 | INFYG | 160 | HISIGDQTDYAESAKG | 168 | LSRIYPYDY |
| 43 | 79 | INFYG | 160 | HISIGDQTDYAESAKG | 169 | LSRIAPYDY |
| 44 | 79 | INFYG | 160 | HISIGDQTDYAESAKG | 170 | LSRIPPYDY |
| 45 | 79 | INFYG | 160 | HISIGDQTDYAESAKG | 171 | LSRIDPYDY |
| 46 | 79 | INFYG | 160 | HISIGDQTDYAESAKG | 172 | LSRIQPYDY |
| 47 | 79 | INFYG | 160 | HISIGDQTDYAESAKG | 173 | LSRIEPYDY |
| 48 | 79 | INFYG | 160 | HISIGDQTDYAESAKG | 174 | LSRIRPYDY |
| 49 | 79 | INFYG | 160 | HISIGDQTDYAESAKG | 175 | LSRIFPYDY |
| 50 | 79 | INFYG | 160 | HISIGDQTDYAESAKG | 176 | LSRISPYDY |
| 51 | 79 | INFYG | 160 | HISIGDQTDYAESAKG | 177 | LSRIGPYDY |
| 52 | 79 | INFYG | 160 | HISIGDQTDYAESAKG | 178 | LSRITPYDY |
| 53 | 79 | INFYG | 160 | HISIGDQTDYAESAKG | 177 | LSRIHPYDY |
| 54 | 79 | INFYG | 160 | HISIGDQTDYAESAKG | 178 | LSRIVPYDY |
| 55 | 79 | INFYG | 160 | HISIGDQTDYAESAKG | 179 | LSRIKPYDY |
| 56 | 79 | INFYG | 160 | HISIGDQTDYAESAKG | 180 | LSRILPYDY |
| 57 | 79 | INFYG | 160 | HISIGDQTDYAESAKG | 181 | LSRIIPYDY |

Unless indicated or defined otherwise, all terms used have their usual meaning in the art, which will be clear to the skilled person. Reference is for example made to the standard handbooks such as Sambrook et al. (1989, Molecular Cloning: A Laboratory Manual (2^{nd} Ed.) Vols. 1-3, Cold Spring Harbor Laboratory Press), F. Ausubel et al. (1987, Current protocols in molecular biology, Green Publishing and Wiley Interscience, New York), Lewin (1985, Genes II, John Wiley & Sons, New York, N.Y.), Old et al. (1981, Principles of Gene Manipulation: An Introduction to Genetic Engineering (2^{nd} Ed.) University of California Press, Berkeley, CA), Roitt et al. (2001, Immunology (6^{th} Ed.) Mosby/Elsevier, Edinburgh), Roitt et al. (2001, Roitt's Essential Immunology (10^{th} Ed.) Blackwell Publishing, UK), and Janeway et al. (2005, Immunobiology (6^{th} Ed.) Garland Science Publishing/Churchill Livingstone, New York), as well as to the general background art cited herein.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews; Presta (2006, Adv. Drug Deliv. Rev. 58 (5-6): 640-56), Levin and Weiss (2006, Mol. Biosyst. 2(1): 49-57), Irving et al. (2001, J. Immunol. Methods 248(1-2): 31-45), Schmitz et al. (2000, Placenta 21 Suppl. A: S106-12), Gonzales et al. (2005, Tumor Biol. 26(1): 31-43), which describe techniques for protein engineering, such as affinity maturation and other techniques for improving the specificity and other desired properties of proteins such as immunoglobulins.

The term “sequence” as used herein (for example in terms like “immunoglobulin sequence”, “antibody sequence”, “variable domain sequence”, “VHH sequence” or “protein sequence”), should generally be understood to include both the relevant amino acid sequence as well as nucleic acids or nucleotide sequences encoding the same, unless the context requires a more limited interpretation.

“Amino acid sequences” are interpreted to mean a single amino acid or an unbranched sequence of two or more amino acids, depending on the context. Nucleotide sequences are interpreted to mean an unbranched sequence of 3 or more nucleotides.

Amino acids are those L-amino acids commonly found in naturally occurring proteins and are listed in Table B-1. Those amino acid sequences containing D-amino acids are not intended to be embraced by this definition. Any amino acid sequence that contains post-translationally modified amino acids may be described as the amino acid sequence that is initially translated using the symbols shown in the Table B-1 with the modified positions; e.g., hydroxylations or glycosylations, but these modifications shall not be shown explicitly in the amino acid sequence. Any peptide or protein that can be expressed as a sequence modified linkages, cross links and end caps, non-peptidyl bonds, etc., is embraced by this definition.

The terms “protein”, “peptide”, “protein/peptide”, and “polypeptide” are used interchangeably throughout the disclosure, and each has the same meaning for purposes of this disclosure. Each term refers to an organic compound made of a linear chain of two or more amino acids. The compound may have ten or more amino acids; twenty-five or more amino acids; fifty or more amino acids; one hundred or more amino acids, two hundred or more amino acids, and even three hundred or more amino acids. The skilled artisan will appreciate that polypeptides generally comprise fewer amino acids than proteins, although there is no art-recognized cut-off point of the number of amino acids that distinguish a polypeptide from a protein; that polypeptides may be made by chemical synthesis or recombinant methods; and that proteins are generally made in vitro or in vivo by recombinant methods as known in the art.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

For instance, when a nucleotide sequence, amino acid sequence or polypeptide is said to “comprise” another nucleotide sequence, amino acid sequence or polypeptide, respectively, or to “essentially consist of” another nucleotide sequence, amino acid sequence or polypeptide, this may mean that the latter nucleotide sequence, amino acid sequence or polypeptide has been incorporated into the first mentioned nucleotide sequence, amino acid sequence or polypeptide, respectively, but more usually this generally means that the first mentioned nucleotide sequence, amino acid sequence or polypeptide comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when a polypeptide of the present technology is said to comprise an immunoglobulin single variable domain, this may mean that said immunoglobulin single variable domain sequence has been incorporated into the sequence of the polypeptide of the present technology, but more usually this generally means that the polypeptide of the present technology contains within its sequence the sequence of the immunoglobulin single variable domains irrespective of how said polypeptide of the present technology has been generated or obtained. Also, when a nucleic acid or nucleotide sequence is said to comprise another nucleotide sequence, the first mentioned nucleic acid or nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the first mentioned, larger nucleic acid or nucleotide sequence).

When an amino acid sequence or polypeptide is said to “essentially consist of” an immunoglobulin single variable domain, it is meant that said amino acid sequence or polypeptide either is exactly the same as the immunoglobulin single variable domain or corresponds to polypeptide or amino acid sequence which has a limited number of amino acid residues, such as 1-20 amino acid residues, for example 1-10 amino acid residues and preferably 1-6 amino acid residues, such as 1, 2, 3, 4, 5 or 6 amino acid residues, added at the amino terminal end, at the carboxy terminal end, or at both the amino terminal end and the carboxyterminal end of the immunoglobulin single variable domain. When “consist of” is used, it is meant that the amino acid sequence or polypeptide is exactly the same as the immunoglobulin single variable domain.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the present technology described herein. Such equivalents are intended to be encompassed by the present technology.

The term “and/or” wherever used herein includes the meaning of “and”, “or” and “all or any other combination of the elements connected by said term”.

The term “about” or “approximately” as used herein means within 20%, preferably within 15%, more preferably within 10%, and most preferably within 5% of a given value or range.

### 6 EXAMPLES

### 6.1 Example 1: Generation and Off-Rate Determination of T Cell Engaging (TCE) ISVDs

### Generation of Variants

Amino acids in positions 61, 99, and 10³ (Kabat numbering) in ISVD T017000700 (SEQ ID NO:1) were substituted to generate different variants, which maintained function and overall developability, but had reduced or no isomerization at position 61 and reduced or no oxidation at sites 99 and/or 10³ (Kabat numbering).

ISVD T017000700 and the generated variant ISVDs are shown in Table A-4 above. All ISVDs were labeled with a FLAG-HIS-tag (FLAG3-HIS6, SEQ ID NO: 77) so that they could be analyzed in further experiments.

### Off-Rate Determination Towards Human TCRαβ-Zipper Protein

Binding to TCRαβ by the TCE ISVD variants was assessed by determining the dissociation rate constant (kd) towards recombinant human TCRαβ-zipper protein, by means of SPR based assays on a ProteOn XPR36 instrument (BioRad Laboratories, Inc.) or a SPR-32 instrument (Bruker Daltonics SPR).

### Setup on ProteOn XPR36

Binding of crude extract monovalent TCE-FLAG3-HIS6 ISVD constructs to human TCRαβ-zipper protein (huTCR(2XN9)-zipper, in-house produced) was probed by Surface Plasmon Resonance (SPR) (Bio-rad Laboratories, Inc., ProteOn XPR36). The target was immobilized on a GLC sensor chip (short matrix, normal capacity) using standard amine coupling chemistry. Crude extracts of ISVD constructs were injected (in duplicate) at a 1:10 dilution to assess the binding parameters. Each ISVD was injected for 120s and dissociation was assessed during 600s.

Data was double referenced by subtracting a reference analyte lane and a blank buffer injection. The off-rate constant (kd) for each interaction was calculated applying the Langmuir 1:1 interaction model using the ProteOn Manager 3.1.0 (Bio-rad Laboratories, Inc., Version 3.1.0.6).

### Setup on SPR-32

Binding of crude extract monovalent TCE-FLAG3-HIS6 ISVD constructs to human TCRαβ-zipper protein (huTCR(2XN9)-zipper, in-house produced) was probed by Surface Plasmon Resonance (SPR) (Bruker Daltonics SPR, SPR-32). The target was immobilized on a high-capacity amine (HCA) sensor chip using standard amine coupling chemistry. Crude extracts of ISVD constructs were injected at a 1:10 dilution to assess the binding parameters. Each ISVD was injected for 120s and dissociation was assessed during 600s.

Data was double referenced by subtracting a reference spot and a blank buffer injection. The off-rate constant (kd) for each interaction was calculated applying the Langmuir 1:1 interaction model using the SPR-32 Analyser Software (Bruker Daltonics SPR, Version 3.2.0.19).

Results of the measurements can be seen in Tables 1-3.

TABLE 1
SPR based off-rate determination of TCE ISVD variants
at position 61 (Kabat numbering) and reference TCE
ISVD construct for human TCRαβ-zipper protein

| | | | | Human TCRαβ-zipper | |
|---|---|---|---|---|---|
| | | | | kd | kd ratio |
| ISVD ID | Res 61 | Res 99 | Res 103 | (1/s) | vs reference |
| T017000946 | A | W | W | 3.8E−04 | 1.0 |
| T017000945 | E | W | W | 3.6E−04 | 1.0 |
| T017000950 | F | W | W | 3.5E−04 | 0.9 |
| T017000943 | H | W | W | 3.3E−04 | 0.9 |
| T017000941 | I | W | W | 3.4E−04 | 0.9 |
| T017000939 | K | W | W | 3.5E−04 | 1.0 |
| T017000948 | L | W | W | 3.3E−04 | 0.9 |
| T017000937 | N | W | W | 3.3E−04 | 0.9 |
| T017000938 | P | W | W | 3.6E−04 | 1.0 |
| T017000949 | Q | W | W | 3.8E−04 | 1.0 |
| T017000940 | R | W | W | 3.8E−04 | 1.0 |
| T017000951 | S | W | W | 3.9E−04 | 1.0 |
| T017000942 | T | W | W | 3.4E−04 | 0.9 |
| T017000944 | V | W | W | 3.8E−04 | 1.0 |
| T017000947 | Y | W | W | 3.5E−04 | 0.9 |
| T017000700 | D | W | W | 3.7E−04 | 1.0 |
| (reference) | | | | | |

As can be seen from Table 1, binding of TCE ISVD variants with a substitution in position 61 (Kabat numbering) towards the human TCRαβ-zipper protein, as assessed by SPR-based off-rate determination, is maintained at levels very similar to reference (T017000700) for all constructs tested, i.e., constructs having amino acid residues A, E, F, H, I, K, L, N, P, Q, R, S, T, V, Y at position 61.

TABLE 2
SPR based off-rate determination of TCE ISVD variants at position
99 and position 61 (Kabat numbering) fixed as E, and reference
TCE ISVD construct for human TCRαβ-zipper protein

| | | | | Human TCRαβ-zipper | |
|---|---|---|---|---|---|
| | | | | kd | kd ratio |
| ISVD ID | Res 61 | Res 99 | Res 103 | (1/s) | vs reference |
| T01700978 | E | Y | W | 4.3E−04 | 1.4 |
| T017000992 | E | A | W | 7.0E−03 | 23 |
| T017000994 | E | D | W | 7.6E−03 | 24 |
| T017000996 | E | E | W | 1.1E−02 | 37 |
| T017000998 | E | F | W | 6.8E−04 | 2 |
| T017001000 | E | G | W | 3.1E−03 | 10 |
| T017001002 | E | H | W | 1.0E−03 | 3 |
| T017001006 | E | I | W | 1.5E−02 | 49 |
| T017001004 | E | K | W | 2.5E−03 | 8 |
| T017001005 | E | L | W | 2.3E−03 | 8 |
| T017000993 | E | P | W | 3.4E−02 | 109 |
| T017000995 | E | Q | W | 1.9E−02 | 61 |
| T017000997 | E | R | W | 1.9E−03 | 6 |
| T017000999 | E | S | W | 3.8E−03 | 12 |
| T017001001 | E | T | W | 4.2E−03 | 14 |
| T017001003 | E | V | W | 6.3E−03 | 20 |
| T017000700 | D | W | W | 3.1E−04 | 1.0 |
| (reference) | | | | | |

As can be seen from Table 2, binding of TCE ISVD variants with a substitution at position 99 (Kabat numbering), towards the human TCRαβ-zipper protein, was tested in constructs where residue 61 was fixed as glutamic acid (E). In the SPR-based off-rate determination, all tested variants, i.e., with residues Y, A, D, E, F, G, H, I, K, L, P, Q, R, S, T, V at position 99, maintained detectable levels of binding towards the human TCRαβ-zipper protein, however, with a range of up to approximately 100-fold faster off-rates when compared to reference T017000700.

TABLE 3
SPR based off-rate determination of TCE ISVD variants
at position 103 (Kabat numbering) and reference TCE
ISVD construct for human TCRαβ-zipper protein

| | | | | Human TCRαβ-zipper | |
|---|---|---|---|---|---|
| | | | | kd | kd ratio |
| ISVD ID | Res 61 | Res 99 | Res 103 | (1/s) | vs reference |
| T017000953 | D | W | A | 2.0E−04 | 0.5 |
| T017000954 | D | W | E | 3.7E−04 | 1.0 |
| T017000965 | D | W | F | 3.8E−04 | 1.0 |
| T017000963 | D | W | G | No binding | No binding |
| T017000957 | D | W | H | 2.7E−04 | 0.7 |
| T017000958 | D | W | I | 2.5E−04 | 0.7 |
| T017000961 | D | W | K | 3.4E−04 | 0.9 |
| T017000956 | D | W | L | 2.4E−04 | 0.6 |
| T017000964 | D | W | P | No binding | No binding |
| T017000959 | D | W | Q | 2.6E−04 | 0.7 |
| T017000952 | D | W | R | 4.7E−04 | 1.3 |
| T017000962 | D | W | S | 3.6E−04 | 1.0 |
| T017000966 | D | W | T | 3.1E−04 | 0.8 |
| T017000960 | D | W | V | 2.5E−04 | 0.7 |
| T017000955 | D | W | Y | 4.4E−04 | 1.2 |
| T017000700 | D | W | W | 3.8E−04 | 1.0 |
| (reference) | | | | | |

As can be seen in Table 3, binding of TCE ISVD variants with a substitution at Kabat position 103 towards the human TCRαβ-zipper protein, as assessed by SPR-based off-rate determination, is maintained at levels very similar to reference T017000700 for constructs having amino acid residues A, E, F, H, I, K, L, Q, R, S, T, V, Y, at position 103, while no binding was observed for constructs having amino acid residues G or P at position 103.

Based on the results of the SPR analyses, it can be concluded that when substituting amino acids at one or more of the positions 61, 99 and 103 (Kabat numbering) in the sequence of reference T017000700, functional ISVDs that bind to TCR can be obtained.

A number of the generated ISVDs was selected and further analyzed in additional experiments, which will be discussed here below.

### 6.2 Example 2: Determination of Biophysical Properties (Tm, Tagg and Oligomerization) of TCE ISVD Variants

Determination of the melting temperature in a thermal shift assay (TSA) The thermal shift assay (TSA) was performed in a 96-well plate on the LightCycler 48011 machine (Roche). Per row, one ISVD was analyzed at the following pH: 4, 5, 6, 7, 8 and 9. Per well, 5 μL of ISVD sample (0.8 mg/mL in PBS) was added to 5 μL of Sypro Orange (40× in MilliQ water; Invitrogen cat. No. S6551) and 10 μL of buffer (100 mM phosphate, 100 mM borate, 100 mM citrate and 115 mM NaCl, pH range 3.5 to 9). The applied temperature gradient (37 to 99° C. at a rate of 0.03° C./s) induces unfolding of the ISVDs, whereby hydrophobic patches become exposed. Sypro Orange binds to those hydrophobic patches, resulting in an increase in fluorescence intensity (Ex/Em=465/580 nm). The inflection point of the first derivative of the fluorescence intensity curve at pH 7 serves as a measure of the melting temperature.

### Onset of Aggregation Temperature Determination (Tagg Assay)

The temperature at which an ISVD protein starts to aggregate (=temperature of onset of aggregation=Tagg) was determined by Dynamic Light Scattering (DLS) using the DynaPro Plate reader (Wyatt). For this, ISVDs with a 3×FLAG-HIS6 tag (SEQ ID NO:77), produced in E. coli, purified via IMAC followed by preparative SEC, filtered (0.22 μm), at a concentration of 1 mg/mL (D-PBS) were used. After thawing, the sample was filtered over a 0.1 μm membrane and centrifuged for 5 min at 14000 rpm. Samples of 30 μL (4 replicates) were heated from 40 to 80° C. at a constant rate of 0.25° C./minute with continuous recording of the light scattering intensities. The Hydrodynamic radius derived from the measured intensities was plotted against the temperature to determine the temperature at which the radius started to increase (=Tagg, ° C.).

### Oligomerization Assay

Oligomerization propensity of monovalent ISVDs under stressed conditions (1 week at 45° C.) was investigated by analytical size exclusion chromatography (SE-HPLC). For this, ISVDs with a 3×FLAG-HIS6 tag, produced in E. coli, purified via IMAC followed by preparative SEC, filtered (0.22 am), at a concentration of 1 mg/mL (DPBS) were used. The SE-HPLC profiles of two 100 μL aliquots were compared: one sample was incubated for 1 week at −20° C. and the other sample for 1 week at 45° C. Samples were cleared by centrifugation for 5 minutes at 20000 RCF and subsequently analyzed on a Acquity UPLC BEH200 SEC (mobile phase 750 mM L-Arginine. HCl+10 mM Phosphate pH 7.0, flowrate 0.4 mL/minute). The difference in relative pre-peak areas of stressed (+45° C.) and non-stressed samples (−20° C.) was calculated and reported as A % oligo (=% oligo 1W 45° C.-% oligo TO).

### Results

Purified variants of the TCE ISVDs harbouring selected amino acid substitutions in positions 61, 99 and 10³ (Kabat numbering) (see Table A-4) were characterized in terms of melting temperature (Tm), onset of aggregation (Tagg) and oligomerization after 1 week at 45° C. as is illustrated in Tables 4 and 5.

In position 61 of the TCE ISVD, amino acid residue variants A, E, P, Q, R, S, V, were selected because these are the most frequently occurring residues at this position in human V_{H} genes.

In position 99 of the TCE ISVD, amino acid residue variants A, H, Q S, T, and Y were selected. Y was selected because it was the residue that affected the off-rate of the respective ISVD variant for binding to the TCRαβ the least (Table 2). Residues A, H, Q S, T were selected because the respective TCE ISVDs represented a range of different off-rates for binding to the TCRαβ.

In approximately 95% of human VH genes and naturally occurring ISVDs [in house analysis of sequences] tryptophan (W) is found in kabat position 103. Other residues that can be found at position 103 of naturally occurring ISVDs are Y, R and S. Hence, Y, R and S were selected for the amino acid residue variants in position 103.

TABLE 4
Biophysical properties (Tm, Tagg and Oligomerization) of TCE ISVD
variants at position 61 and 99, and reference TC017000700

| ISVD ID | Tm | ΔTm (° C.) vs ref | Tagg | Δtagg vs ref (° C.) | Δ % oligo | Δ % oligo vs ref |
|---|---|---|---|---|---|---|
| T017000700 | 72.0 | — | 68 | — | 0.2 | — |
| T017000938 | 72.0 | 0.0 | 66 | −2 | 1.0 | 0.8 |
| T017000940 | 68.6 | −3.4 | 60 | −8 | 0.9 | 0.7 |
| T017000944 | 72.0 | 0.0 | 61 | −7 | 0.9 | 0.7 |
| T017000945 | 72.5 | 0.5 | 68 | 0 | 0.6 | 0.4 |
| T017000946 | 70.0 | −2.0 | 64 | −4 | 0.7 | 0.5 |
| T017000949 | 69.6 | −2.4 | 64 | −4 | 1.1 | 0.9 |
| T017000951 | 70.0 | −2.0 | 63 | −5 | 1.0 | 0.8 |
| T017000967 | 72.0 | 0.0 | 73 | 5 | 0.4 | 0.2 |
| T017000975 | 73.4 | 1.4 | 71 | 3 | 1.5 | 1.3 |
| T017000976 | 69.6 | −0.4 | 64 | −4 | 0.9 | 0.7 |
| T017000977 | 70.5 | −1.5 | 65 | −3 | 1.6 | 1.4 |
| T017000978 | 73.0 | 1.0 | 71 | 3 | 0.0 | −0.2 |
| T017000979 | 71.5 | −0.5 | 68 | 0 | 1.7 | 1.5 |
| T017000980 | 70.5 | −1.5 | 67 | −1 | 0.1 | −0.1 |
| T017000981 | 70.5 | −1.5 | 68 | 0 | 1.3 | 1.1 |
| T017000992 | 73.0 | 1.0 | 73 | 5 | 0.2 | 0.0 |
| T017000995 | 71.0 | −1.0 | 73 | 5 | 0.1 | −0.1 |
| T017000999 | 72.0 | 0.0 | 72 | 4 | 0.1 | −0.1 |
| T017001001 | 71.0 | −1.0 | 68 | 0 | 0.3 | 0.1 |
| T017001002 | 72.0 | 0.0 | 69 | 1 | 0.2 | 0.0 |

TABLE 5
Biophysical properties (Tm, Tagg and Oligomerization) of TCE
ISVD variants at position 103, and reference TC017000700

| | Tm | ΔTm (° C.) | Tagg | ΔTagg vs | |
|---|---|---|---|---|---|
| ISVD ID | (° C.) | vs ref | (° C.) | ref (° C.) | Δ% oligo |
| T017000982 | 64.7 | −7.3 | 61 | 7 | 0.1 |
| T017000955 | 63.2 | −8.8 | 57 | 11 | 0.3 |
| T017000952 | 56.9 | −15.1 | 48 | 20 | 0.2 |
| T017000962 | 59.3 | −12.7 | 53 | 15 | 0.3 |

As can be seen from Table 4, T017000978, T0170009921, T017000999 and T017001002 have maintained or improved all three properties as compared to reference T017000700. For Tm a 0° C. to V° C. increase was observed, for Tagg a V° C. to SoC increase was observed and a reduction in the oligomeric fraction from 0.2% for the reference to 0.0% or 0.1% for the ISVDs according to the present technology was observed.

Surprisingly, as can be seen in Table 5, even though the TCR binding of the ISVD variants at position 103 was not affected, substitution in this position resulted in a reduction of the melting temperature by 7.3° C. to 15.1° C., and a reduction of the onset temperature of aggregation by 7° C. to 20° C. as compared to the reference ISVD. This suggests that W103 may be important for biophsyical stability of the TCE ISVDs.

Consequently, moving forward it was decided to keep a tryptophan at position 103, while varying the amino acid residues at positions 61 and 99.

### 6.3 Example 3: Affinity Determination of TCE ISVD Variants Towards Human and Cynomolgus TCRαβ Protein

Binding of purified, monovalent TCE-FLAG3-HIS6 and TCE-HIS6 ISVD constructs to human TCRαβ-zipper protein (huTCR(2XN9)-zipper, in-house produced) and cynomolgus TCRαβ-zipper protein (cyTCR(AEA41865)-zipper, in-house produced) was probed by Surface Plasmon Resonance (SPR) (Bio-rad Laboratories, Inc., ProteOn XPR36). Both targets were immobilized on a GLC sensor chip (short matrix, normal capacity) using standard amine coupling chemistry. ISVD constructs were injected at 6 different concentrations in a multi-cycle kinetics (MCK) experiment. Each concentration of the ISVDs was injected for 120s and dissociation was assessed during 600s.

Data was double referenced by subtracting a reference analyte lane and a blank buffer injection. Affinity constants (ka, kd and K_{D}) were calculated applying the Langmuir 1:1 interaction model using the ProteOn Manager 3.1.0 (Bio-rad Laboratories, Inc., Version 3.1.0.6).

The results of the affinity measurement of TCE ISVD amino acid variants to human and cynomolgus TCRαβ-zipper protein are summarized in Tables 6 and 7 below. As a reference T017000700 was used.

TABLE 6
SPR based kinetic determination of TCE ISVD variants, and reference
T017000700 for binding human TCRαβ-zipper fusion proteins

Human TCRαβ-zipper

| | | | | K_{D} ratio |
|---|---|---|---|---|
| ISVD ID | ka (1/Ms) | kd (1/s) | K_{D} (M) | vs ref |
| T017000700 | 1.3E+05 | 3.7E−04 | 2.5E−09 | 1.0 |
| T017000938 | 1.7E+05 | 3.6E−04 | 2.1E−09 | 0.9 |
| T017000940 | 2.0E+05 | 3.8E−04 | 1.9E−09 | 0.8 |
| T017000944 | 1.6E+05 | 3.8E−04 | 2.3E−09 | 0.9 |
| T017000945 | 1.6E+05 | 3.6E−04 | 2.3E−09 | 0.9 |
| T017000946 | 1.7E+05 | 3.8E−04 | 2.3E−09 | 0.9 |
| T017000949 | 1.8E+05 | 3.8E−04 | 2.1E−09 | 0.9 |
| T017000951 | 1.6E+05 | 3.9E−04 | 2.5E−09 | 1.0 |
| T017000967 | 1.1E+05 | 4.8E−04 | 4.4E−09 | 1.8 |
| T017000975 | 1.4E+05 | 5.5E−04 | 3.8E−09 | 1.5 |
| T017000976 | 1.5E+05 | 5.7E−04 | 3.9E−09 | 1.6 |
| T017000977 | 1.1E+05 | 5.2E−04 | 4.5E−09 | 1.8 |
| T017000978 | 1.1E+05 | 5.2E−04 | 5.0E−09 | 2.0 |
| T017000979 | 1.1E+05 | 5.6E−04 | 4.9E−09 | 2.0 |
| T017000980 | 1.3E+05 | 5.5E−04 | 4.1E−09 | 1.7 |
| T017000981 | 1.2E+05 | 5.3E−04 | 4.4E−09 | 1.8 |
| T017000995 | 3.3E+04 | 3.3E−02 | 1.0E−06 | 404 |
| T017000999 | 3.8E+04 | 5.3E−03 | 1.4E−07 | 56 |
| T017001001 | 2.5E+04 | 8.6E−03 | 3.5E−07 | 141 |

TABLE 7
SPR based kinetic determination of TCE ISVD variants,
and reference T017000700 for binding cynomolgus
TCRαβ-zipper fusion proteins

| | CynoTCRαβ-zipper | | | | |
|---|---|---|---|---|---|
| | | | | K_{D} ratio | K_{D} ratio |
| ISVD ID | ka (1/Ms) | kd (1/s) | K_{D} (M) | vs ref | cy/huTCR |
| T017000700 | 2.2E+05 | 1.8E−03 | 8.0E−09 | 1.0 | 3.3 |
| T017000938 | 2.8E+05 | 1.9E−03 | 6.7E−09 | 0.8 | 3.2 |
| T017000940 | 3.2E+05 | 1.9E−03 | 6.1E−09 | 0.8 | 3.2 |
| T017000944 | 2.8E+05 | 1.9E−03 | 7.0E−09 | 0.9 | 3.0 |
| T017000945 | 2.6E+05 | 1.8E−03 | 7.1E−09 | 0.9 | 3.1 |
| T017000946 | 2.7E+05 | 1.9E−03 | 7.0E−09 | 0.9 | 3.1 |
| T017000949 | 2.8E+05 | 1.9E−03 | 6.8E−09 | 0.8 | 3.2 |
| T017000951 | 2.6E+05 | 2.0E−03 | 7.4E−09 | 0.9 | 3.0 |
| T017000967 | 2.0E+05 | 2.8E−03 | 1.4E−08 | 1.7 | 3.1 |
| T017000975 | 2.3E+05 | 3.4E−03 | 1.5E−08 | 1.8 | 3.9 |
| T017000976 | 2.3E+05 | 3.5E−03 | 1.5E−08 | 1.9 | 3.9 |
| T017000977 | 1.8E+05 | 3.5E−03 | 1.9E−08 | 2.4 | 4.3 |
| T017000978 | 1.7E+05 | 3.3E−03 | 2.0E−08 | 2.5 | 4.0 |
| T017000979 | 1.8E+05 | 3.5E−03 | 1.9E−08 | 2.3 | 3.8 |
| T017000980 | 2.1E+05 | 3.4E−03 | 1.7E−08 | 2.1 | 4.0 |
| T017000981 | 2.1E+05 | 3.5E−03 | 1.7E−08 | 2.1 | 3.8 |
| T017000995 | 4.7E+04 | 9.3E−02 | 2.0E−06 | 246 | 2.0 |
| T017000999 | 5.1E+04 | 1.8E−02 | 3.5E−07 | 43 | 2.5 |
| T017001001 | 3.2E+04 | 3.4E−02 | 1.1E−06 | 133 | 3.1 |

As can be seen from Tables 6 and 7, the affinity of the TCE ISVD variants remains comparable to the reference TCE ISVD (K_{D} values within 2.5-fold), for all tested constructs, with the exception of T017000995, T017000999, and T017001001.

Each ISVD shows a 2-to 4-fold higher K_{D} on cynomolgus TCRαβ protein than on human TCRαβ protein. This shows that the ISVDs according to the present technology have potential to be developed and used in human therapeutic applications.

### 6.4 Example 4: Binding of Monovalent TCE ISVDs to Purified Primary Human T Cells

TCE ISVDs according to the present technology were tested for their binding to human T cells in flow cytometry. The following ISVDs were tested: T017000700 (reference), T017000978, T017000995, T017000999, and T017001001. Negative controls were also added. Unstained cells (US), cells stained only with GaM-PE and cells stained only with ANTI-FLAG® M2 antibody and GaM-PE (a-FLAG+GaM-PE) were included as negative controls.

In brief, cells were harvested and transferred to a V-bottom 96-well plate (5×1E4 cells per well in 50 μL) and incubated with a serial dilution of TCE ISVDs for 3.5 hours at 4° C. in FACS buffer (D-PBS (Gibco, 14190) with 2% FBS (Sigma, F7524) and 0.05% sodium azide (Acros organics, 19038)).

Next, cells were washed 3 times with FACS buffer and incubated with 1 μg/mL Monoclonal ANTI-FLAG® M2 antibody (Sigma F1804) for 30 min at 4° C., washed again, and incubated for 30 min at 4° C. with 1/100 diluted R-Phycoerythrin-conjugated AffiniPure F(ab′)2 Fragment Goat Anti-mouse IgG, Fc γ Fragment Specific (Jackson Immunoresearch, 115-116-071; referred to as GaM-PE).

Subsequently, cells were resuspended in FACS buffer supplemented with 5 nM TO-PRO®-3 Iodide (642/661) (Life Technologies-Molecular Probes, T3605) to distinguish live from dead cells. After staining, cells were analyzed using MACSQuant® Flow cytometer (Miltenyi) using FlowLogic Software. First a P1 population which represented more than 80% of the total cell population was selected based on FSC-SSC distribution. From this population (P1) the TO-PRO®-3 Iodide positive (dead) cells were excluded, and the mean fluorescence intensity PE value was calculated.

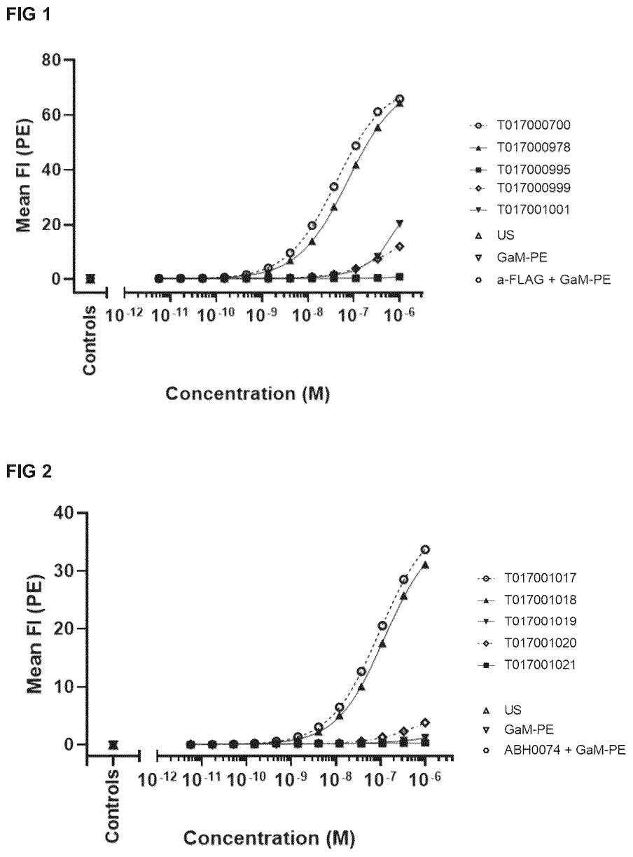

Results of the measurements can be seen in FIG. 1. As expected, no staining of the human T cells was observed for the control conditions without the TCE ISVDs. Additionally, as can been seen from this Figure, based on the mean fluorescence intensity measured, T017000978 has comparable staining to the reference T017000700. Variants T017000995, T017000999, and T017001001 have much lower staining, which corresponds to the binding affinities determined on the zipper protein as shown in Table 6.

### 6.5 Example 5: Generation of TCE-CD123-ALB Constructs

TCE ISVD variants were formatted into multispecific ISVD constructs with a tumor anchoring ISVD building block directed against CD123 and an ISVD building block directed against human serum albumin linked by 9GS linkers. The TCE variant building block was placed either in the N-terminal position (position 1) or in the second position (position 2); the anti-CD123 building block then was placed either in position 2, or position 1, respectively. In all constructs, the anti-human serum albumin building block was placed at the C-terminal position 3. In addition to the substitutions in the TCE building block at positions 61 and 99 (Kabat numbering), the first residue of said TCE building block was either maintained as E or changed to D, when the TCE building block was placed at position 1 in the multispecific construct. The E1D mutation is commonly introduced to avoid pyroglutamate formation. The generated constructs are listed in Table 8 below. The reference construct T017001017 comprised the reference TCR binding ISVD T017000700.

TABLE 8
List of TCE-CD123-ALB and CD123-TCE-ALB constructs

| CD123-TCE-ALB/TCE- | | anti-TCR ISVD | | Kabat position variant of TCE | | | |
|---|---|---|---|---|---|---|---|
| CD123-ALB | | in the construct | | in the construct | | | |
| Sample | SEQ | | TCE position | | | Res | Res |
| ID | ID NO | Name | in construct | Res1 | Res61 | 99 | 103 |
| T017001017 | 58 | T017000700 | 1 | E | D | W | W |
| T017001018 | 59 | T017000978 | 1 | E | E | Y | W |
| T017001019 | 60 | T017001001 | 1 | E | E | T | W |
| T017001020 | 61 | T017000999 | 1 | E | E | S | W |
| T017001021 | 62 | T017000995 | 1 | E | E | Q | W |
| T017001022 | 63 | T017000975 | 1 | D | D | W | W |
| T017001023 | 64 | T017000991 | 1 | D | E | Y | W |
| T017001024 | 65 | T017001001 | 1 | D | E | T | W |
| | | (E1D) | | | | | |
| T017001025 | 66 | T017000999 | 1 | D | E | S | W |
| | | (E1D) | | | | | |
| T017001026 | 67 | T017000995 | 1 | D | E | Q | W |
| | | (E1D) | | | | | |
| T017001027 | 68 | T017000700 | 2 | E | D | W | W |
| T017001028 | 69 | T017000978 | 2 | E | E | Y | W |
| T017001029 | 70 | T017001001 | 2 | E | E | T | W |
| T017001030 | 71 | T017000999 | 2 | E | E | S | W |
| T017001031 | 72 | T017000995 | 2 | E | E | Q | W |

The following TCE ISVD building blocks were used in the above listed constructs:
For T017001017 and T017001027, the reference TCE ISVD T017000700 (SEQ ID NO: 1) was used.
For T017001018 and T017001028, T017000978 (SEQ ID NO: 37) was used.
For T017001019, T017001024, and T017001029, T017001001 (SEQ ID NO: 52) was used. T017001024 further contained the E1D mutation in the TCE ISVD.
For T017001020, T017001025, and T017001030, T017000999 (SEQ ID NO: 50) was used. T017001025 further contained the E1D mutation in the TCE ISVD.
For T017001021, T017001026, and T017001031, T017000995 (SEQ ID NO 46) was used. T017001026 further contained the E1D mutation in the TCE ISVD.
For T017001022, T017000975 (SEQ ID NO: 34) was used. This is T017000700 with an E1D mutation.
For T017001023, T017000991 (SEQ ID NO: 42) was used. This is T017000978 with an E1D mutation.

### 6.6 Example 6: Affinity Determination of TCE-CD123-ALB Constructs Towards Human and Cynomolgus TCRαβ-Zipper Protein

Binding of purified TCE-CD123-ALB and CD123-TCE-ALB ISVD constructs to human TCRαβ-zipper protein (huTCR(2XN9)-zipper, in-house produced) and cynomolgus TCRαβ-zipper protein (cyTCR(AEA41865)-zipper, in-house produced) was probed by Surface Plasmon Resonance (SPR) (Cytiva, Biacore 8K+). Both targets were immobilized on a CM5 sensor chip using standard amine coupling chemistry. In total 15 ISVD constructs (Table 8) were injected at 12 different concentrations (serial dilution from 20 μM to 0.84 nM) in a multi-cycle kinetics experiment. Each concentration of the ISVDs was injected for 180s and dissociation was assessed during 600s.

Data was double referenced by subtracting a reference flow cell (FC) and a blank buffer injection. Affinity constants (ka, kd and K_{D}) were calculated applying the Langmuir 1:1 interaction model using the Biacore Insight Evaluation Software (Cytiva, Version 3.0.12.15655). The results of the affinity measurement of the 15 multivalent ISVD constructs (Table 8) on human TCRαβ protein are summarized in Table 9 and on the cynomolgus TCRαβ protein in Table 10.

TABLE 9
Affinity determination of TCE-CD123-ALB constructs
towards human TCRαβ-zipper protein

| | Kinetics on human TCR-zipper protein | | | |
|---|---|---|---|---|
| | | | | Fold change |
| Sample ID | ka (1/Ms) | kd (1/s) | K_{D} (M) | K_{D} to T017001017 |
| T017001017 | 7.7E+04 | 3.3E−04 | 4.3E−09 | 1 |
| T017001018 | 6.1E+04 | 4.6E−04 | 7.6E−09 | 2 |
| T017001019 | 1.4E+04 | 6.9E−03 | 5.0E−07 | 116 |
| T017001020 | 2.3E+04 | 4.6E−03 | 2.0E−07 | 47 |
| T017001021 | 1.8E+04 | 2.7E−02 | 1.5E−06 | 360 |
| T017001022 | 8.5E+04 | 4.4E−04 | 5.2E−09 | 1 |
| T017001023 | 7.2E+04 | 6.6E−04 | 9.1E−09 | 2 |
| T017001024 | 1.8E+04 | 1.5E−02 | 8.7E−07 | 205 |
| T017001025 | 2.5E+04 | 7.0E−03 | 2.8E−07 | 65 |
| T017001026 | 2.5E+04 | 4.3E−02 | 1.8E−06 | 412 |
| T017001027 | 1.3E+04 | 5.9E−04 | 4.5E−08 | 11 |
| T017001028 | 1.1E+04 | 6.9E−04 | 6.3E−08 | 15 |
| T017001029 | 3.6E+03 | 1.4E−02 | 3.8E−06 | 897 |
| T017001030 | 6.0E+03 | 5.6E−03 | 9.2E−07 | 216 |
| T017001031 | 5.5E+03 | 3.9E−02 | 7.0E−06 | 1654 |

TABLE 10
Affinity determination of TCE-CD123-ALB constructs
towards cynomolgus TCRαβ-zipper protein

| | Kinetics on cynomolgus TCR-zipper protein | | | | |
|---|---|---|---|---|---|
| | | | | Fold change K_{D} to | K_{D} ratio |
| Sample ID | ka (1/Ms) | kd (1/s) | K_{D} (M) | T017001017 | cy/huTCR |
| T017001017 | 1.2^{E}+05 | 1.5^{E}−03 | 1.3^{E}−08 | 1 | 2.9 |
| T017001018 | 1.2^{E}+05 | 3.9^{E}−03 | 3.2^{E}−08 | 3 | 4.2 |
| T017001019 | 1.7^{E}+04 | 2.8^{E}−02 | 1.6^{E}−06 | 129 | 3.3 |
| T017001020 | 2.8^{E}+04 | 1.6^{E}−02 | 5.5^{E}−07 | 44 | 2.8 |
| T017001021 | 2.4^{E}+04 | 8.2^{E}−02 | 3.4^{E}−06 | 274 | 2.2 |
| T017001022 | 1.5^{E}+05 | 2.3^{E}−03 | 1.5^{E}−08 | 1 | 2.9 |
| T017001023 | 1.9^{E}+05 | 7.0^{E}−03 | 3.7^{E}−08 | 3 | 4.1 |
| T017001024 | 2.2^{E}+04 | 5.1^{E}−02 | 2.4^{E}−06 | 189 | 2.7 |
| T017001025 | 3.0^{E}+04 | 2.3^{E}−02 | 7.8^{E}−07 | 63 | 2.8 |
| T017001026 | 2.9^{E}+04 | 1.1^{E}−01 | 3.9^{E}−06 | 313 | 2.2 |
| T017001027 | 3.0^{E}+04 | 5.8^{E}−03 | 1.9^{E}−07 | 15 | 4.3 |
| T017001028 | 1.6^{E}+04 | 6.5^{E}−03 | 4.0^{E}−07 | 32 | 6.4 |
| T017001029 | 5.3^{E}+03 | 4.8^{E}−02 | 9.1^{E}−06 | 728 | 2.4 |
| T017001030 | 7.5^{E}+03 | 2.7^{E}−02 | 3.6^{E}−06 | 286 | 3.9 |
| T017001031 | 8.7^{E}+03 | 1.5^{E}−01 | 1.8^{E}−05 | 1402 | 2.5 |

Tables 9 and 10 show that the TCE ISVD variants according to the present technology, when used in a construct, retain their ability to bind to the TCR. This shows that the TCE ISVD variants according to the present technology can be used in combination with a targeting ISVD. Therefore, they are suitable for development and use in target-specific therapeutic applications.

### 6.7 Example 7: Determination of the Binding by TCE-CD123-ALB ISVD Constructs to Purified Primary Human T Cells

Dose dependent binding by the TCE-CD123-ALB ISVD constructs according to the present technology on primary human T cells was determined using flow cytometry. The following constructs were used: T017001017 (reference), T017001018, T017001019, T017001020, and T017001021. Unstained cells (US), cells stained only with GaM-PE and cells stained only with ABH0074 and GaM-PE (ABH0074+GaM-PE) were included as negative controls.

In brief, cells were harvested and transferred to a V-bottom 96-well plate (5×1E4 cells per well in 50 μL) and incubated with a serial dilution of TCE-CD123-ALB ISVD constructs for 2.5 hours at 4° C. in FACS buffer (D-PBS (Gibco, 14190) with 2% FBS (Sigma, F7524) and 0.05% sodium azide (Acros organics, 19038)).

Next, cells were washed 3 times with FACS buffer and incubated with 10 μg/mL anti-VHH mAb (prepared in-house) for 30 min at 4° C., washed again, and incubated for 30 min at 4° C. with 1/100 diluted R-Phycoerythrin-conjugated AffiniPure F(ab′)2 Fragment Goat Anti-mouse IgG, Fc γ Fragment Specific (Jackson Immunoresearch, 115-116-071). Subsequently, cells were resuspended in FACS buffer supplemented with 5 nM TO-PRO®-3 Iodide (642/661) (Life Techn.-Molecular Probes, T3605) to distinguish live from dead cells. After staining, cells were analyzed using MACSQuant® Flow cytometer (Miltenyi) using FlowLogic Software. First a P1 population which represented more than 80% of the total cell population was selected based on FSC-SSC distribution. From this population (P1) the TO-PRO®-3 Iodide positive (dead) cells were excluded, and the mean fluorescence intensity PE value was calculated.

Results of the measurements are shown in FIG. 2. As expected, no staining of the human T cells was observed for the control conditions without the TCE ISVDs. Additionally, as can be seen from this Figure, T017001018 (comprising TCE ISVD T017000978) and the reference T017001017 (comprising TCE ISVD T017000700) bind with a similar EC50 to human T cells. T017001019 (comprising TCE ISVD T017000995), T017001020 (comprising TCE ISVD T017000999), and T017001021 (comprising TCE ISVD T017001001), on the other hand have lower binding affinities for human T cells. This corresponds to the binding observed on the zipper proteins as shown in Table 9.

### 6.8 Example 8: Multispecific TCE-CD123-ALB Constructs Induce Human T Cell Mediated Target Cell Killing

### Flow Cytometry-Based Cytotoxicity Assay

The ISVD constructs according to the present technology were further characterized for redirected T cell mediated killing in a flow cytometry-based cytotoxicity assay using human primary T cells as effector cells and non-adherent target cells. Target cells were labelled with 4 μM PKH26 membrane dye using the PKH26 red fluorescent cell linker kit (Sigma, PKH26GL-1KT) according to manufacturer's instructions. Effector cells (2.5×105 cells/well) and PKH26 labelled target cells (2.5×104 cells/well) were co-incubated in 96-well V-bottom plates (Greiner Bio-one, #651 180) (effector versus target ratio of 10:1) in assay medium of the target cell line (target growth medium with 1% Penicillin/streptomycin (Life Technologies, 15140) and 30 μM Alburex HSA (CSL Behring, 2160-679)). For analysis of concentration dependent cell lysis, serial dilutions of ISVD constructs in target assay medium were added to the cells and incubated for 18 h in a 5% CO2 atmosphere at 37° C. After incubation, cells were pelleted by centrifugation and washed with FACS buffer (D-PBS (Gibco, 14190) with 10% FBS (Sigma, F7524) and 0.05% sodium azide (Acros organics, 19038)). Subsequently, cells were resuspended in 100 μL FACS buffer supplemented with 5 nM TO-PRO®-3 Iodide (642561) (ThermoFisher Scientific, T3605) to distinguish live from dead cells. Cells were analyzed using a MACSQuant X flow cytometer (Miltenyi Biotec). Per sample, a total sample volume of 70 μL was acquired. Gating was set on PKH26 positive cells, and within this population, the TO-PRO®-3 positive cells were determined. The percent specific lysis=((% TO-PRO-3+no construct−% TO-PRO-3+with construct)/% TO-PRO-3+no construct))×100. The assay was run in the presence of an excess amount of HSA, for the ISVDs to be fully saturated with HSA as described above.

### Results

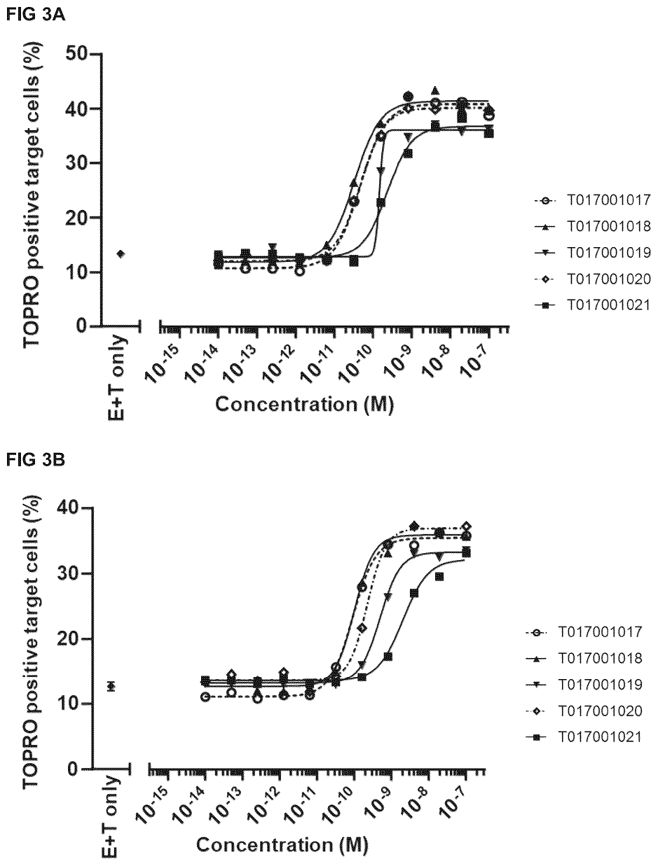

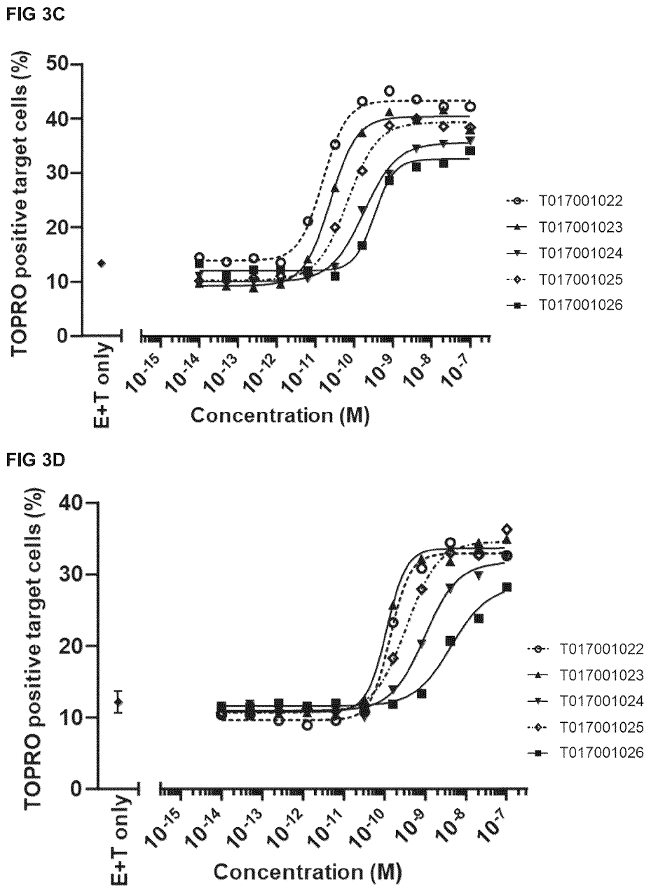

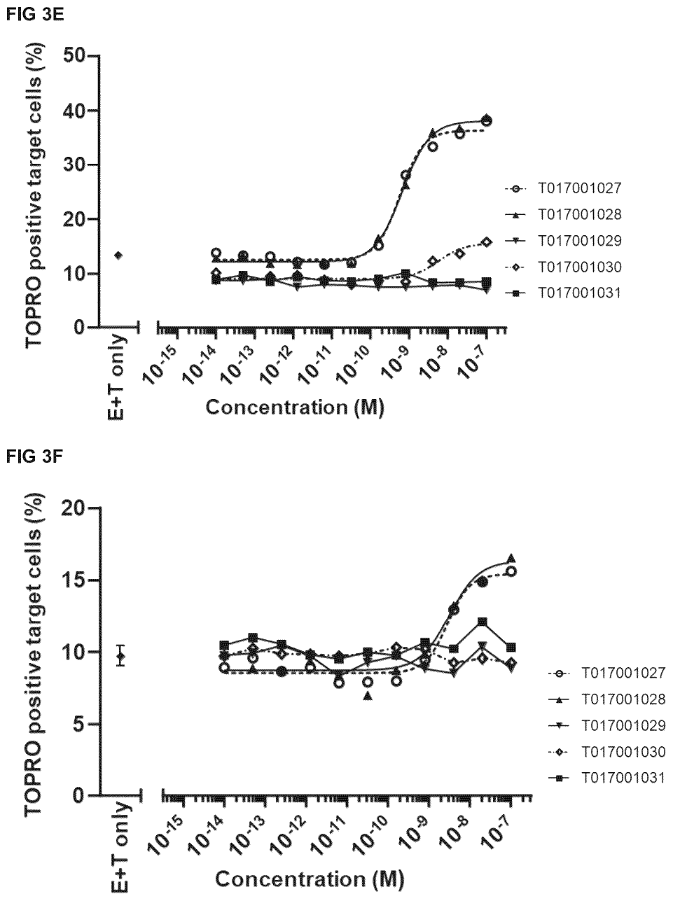

To assess the functionality of the TCE ISVD variants as T cell engagers, the TCE-CD123-ALB formats listed in Table 8 were evaluated in a flow cytometry-based T cell mediated MOLM-13 cell killing assay using human primary T cells (two donors) in combination with CD123 expressing human MOLM-13 target cell line in the presence of 30 μM HSA as described above. Graphical illustration of these results is shown in FIGS. 3A-3F. Calculated EC50 values for the target cell killing are shown in Table 11.

TABLE 11
EC50 (M) of TCE-CD123-ALB constructs in the flow cytometry-based
human T cell mediated MOLM-13 cell killing assay using an effector
to target ratio of 10 to 1 in the presence of 30 μM HSA

MOLM-13 depletion

| | TCE position | Donor #1 | Donor #2 |
|---|---|---|---|
| Sample ID | in construct | EC50 (M) | EC50 (M) |
| T017001017 | 1 | 4.5E−11 | 9.2E−11 |
| T017001018 | 1 | 3.3E−11 | 1.0E−10 |
| T017001019 | 1 | 1.5E−10 | 5.2E−10 |
| T017001020 | 1 | 4.7E−11 | 2.3E−10 |
| T017001021 | 1 | 2.3E−10 | 2.1E−09 |
| T017001022 | 1 | 1.5E−11 | 1.4E−10 |
| T017001023 | 1 | 2.4E−11 | 1.1E−10 |
| T017001024 | 1 | 1.8E−10 | 9.8E−10 |
| T017001025 | 1 | 6.6E−11 | 3.4E−10 |
| T017001026 | 1 | 3.4E−10 | 4.4E−09 |
| T017001027 | 2 | 5.7E−10 | 2.8E−09 |
| T017001028 | 2 | 6.7E−10 | 3.2E−09 |
| T017001029 | 2 | Negative | Negative |
| T017001030 | 2 | 5.8E−09 | Negative |
| T017001031 | 2 | Negative | Negative |

As can be seen from Table 11 and FIGS. 3A-3F, all constructs in which the TCE building block was in the N-terminal position (position 1) mediated potent cell killing of the target cells. Additionally, the mutation in the first position of the TCE ISVD from glutamic acid to aspartic acid did not significantly affect the killing potency of the construct. Furthermore, it seems having the TCE ISVD in the N-terminal position of the construct results in increased potency when compared to placing the same TCE ISVD in the second position in the construct.

Surprisingly, the ISVD constructs T017001019, T017001020 and T017001021, which contain TCE ISVDs, T017001001, T01700999, and T017000995 respectively, with a lower affinity for the TCR zipper protein and human T cells than the other constructs, have a high potency for target cell killing. In fact, even though the affinity observed on human TCR zipper protein as well as on human T cells was more than 40-fold lower than the reference (see Tables 6 and 9 and FIGS. 1-2), the potency for target cell killing was not significantly different.

It has been reported that a low affinity towards the T cell receptor, as compared to the affinity for the tumor-associated target, is important for distribution to the tumor tissue versus the T cell rich secondary lymphoid tissues. Preferential targeting of the tumor tissue is desired to reduce T cell-mediated target clearance.

These particular ISVD constructs with low affinity for the TCR and high potency for target cell killing may thus offer a unique possibility for generating T cell engaging biotherapeutics which maintain high potency, but that can be dosed at lower levels due to higher tumor tissue exposure, which in turn may reduce toxicity risks.

In conclusion, the TCE ISVD variants according to the present technology are very suitable for use in a construct for target-specific therapeutic application in humans.

### 6.9 Example 9: Multispecific TCE-CD123-ALB Construct-Induced Human and Cynomolgus T Cell Mediated Target Cell Killing of KG-1a Cells

In order to assess whether the TCE-CD123-ALB ISVD constructs were able to kill tumor cells, cytotoxicity assays were performed with isolated human or cynomolgus T cells as effector cells. ISVD constructs T017001017, T017001018, T017001019, T017001020, and T017001021 were tested. T017000968 (SEQ ID NO: 75) was included as a negative control. T017000968 comprises the anti-TCR ISVD T017000975 and an albumin binding ISVD, but no anti-CD123 ISVD.

### Redirected T Cell Mediated Killing of CD123 Target Cells by TCE-CD123-ALB Constructs in a Flow Cytometry-Based Assay

Human T cells were collected from Buffy Coat blood from healthy volunteers (Blood bank Gent) using RosetteSep (StemCell Technologies, 15061) followed by enriching on Ficoll-Paque™ PLUS (GE Healthcare, 17-1440-03) according to manufacturer's instructions. The quality and purity of the purified human T cells was checked with anti-CD3 (eBioscience, 12-0037-73), anti-CD8 (BDBiosciences, 555367), anti-CD4 (BD Biosciences, 345771), anti-CD45RO (BD Biosciences, 555493), anti-CD45RA (BDBiosciences, 550855), anti-CD19 (BDBiosciences, 555413), anti-CD25 (BDBiosciences, 557138) and anti-CD69 (BDBiosciences, 557050) fluorescently labelled antibodies in a flow cytometric assay. Cells were frozen in liquid nitrogen.

T cells from cynomolgus monkey (Macaca fascicularis) were isolated in house (Sanofi, Montpellier, France) from PBMC (isolated via Ficoll density centrifugation) using the pan T cell isolation kit (Miltenyi, 130-091-993) according to the manufacturer's instruction. Cells were frozen in liquid nitrogen.

Human CD123 expressing KG1a cells were labelled with 4 μM PKH-26 membrane dye using the PKH26 red fluorescent cell linker kit (Sigma, PKH26GL-1KT) according to manufacturer's instruction and used as target cells. 2.5×1E5 effector (i.e. Human or cynomolgus primary T cells) and 2.5×1E4 target cells (i.e. PKH-labelled KG1a cells) were co-incubated in 96-well V-bottom plates (effector versus target ratio of 10:1). For measurement of the concentration-dependent cell lysis, serial dilutions of the TCE-CD123-ALB constructs in assay medium with 30 μM HSA (CSL Behring, Alburex 20 human serum albumin) were added to the cells and incubated for 18 hours in a 5% CO2 atmosphere at 37° C.

After incubation, cells were pelleted by centrifugation and resuspended in FACS buffer supplemented with 5 nM TO-PRO®-3 Iodide (642/661) (Life Techn.-Molecular Probes, T3605) to distinguish live from dead cells. Cells were analyzed using a MACSQuant© Flow cytometer (Miltenyi). Gating was set on PKH26 positive target cells, and within this population the percentage of TO-PRO®-3 Iodide positive cells was determined.

### Results

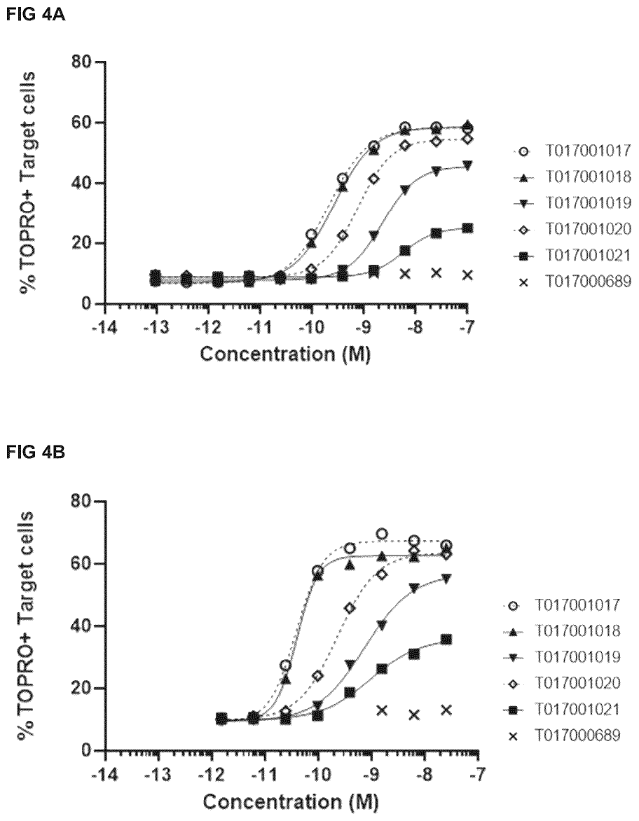

The results can be seen below in Table 12 and in FIGS. 4A-4B.

TABLE 12
TCE-CD123-ALB constructs in the flow cytometry-
based T cell mediated KG-1a cell killing assay

| | Potency (M) - | Potency (M) - |
|---|---|---|
| Sample ID | human T cells | cyno T cells |
| T017001017 | 2.1E−10 | 3.9E−11 |
| T017001018 | 2.7E−10 | 4.0E−11 |
| T017001019 | 2.3E−09 | 7.4E−10 |
| T017001020 | 7.6E−10 | 2.3E−10 |
| T017001021 | 5.7E−09 | 9.4E−10 |
| T017000689 | — | — |

As can be seen from Table 12 and FIGS. 4A-4B, all TCE ISVD constructs were potent in killing KG-1a cells with both human and cyno T cells. As expected, the negative control construct T017000689 showed no cell killing.

The low affinity TCE ISVDs T017001019, T017001020, and T017001021 also had high potency in the KG1-a cell killing assay. This further supports that these TCE constructs with low affinity anti-TCR building blocks have significant potential as T cell engaging biotherapeutics, as was mentioned in Example 8.

Furthermore, for all tested TCE-CD123-ALB formats, the mediated cytotoxic activity observed for each of the formats using human T cells as effector cells was very similar to the activity mediated by cynomolgus T cells.

This cell killing assay thus further supports that the TCE ISVD variants according to the present technology are very suitable for use in a construct for target-specific therapeutic application in humans.

### 6.10 Example 10: Functionality of the TCE-GPC3-GPC3-ALB Fusion

### Generation of TCE-GPC3-GPC3-ALB Fusion

TCE ISVD variants were formatted into multispecific ISVD constructs with two tumor anchoring ISVD building blocks directed against glypican-3 (GPC3) and an ISVD building block directed against human serum albumin linked by 5GS and 9GS linkers. The first residue of the TCE building block was changed to D. The generated constructs are listed in Table 13 below. The reference construct (A022600427) comprised the reference TCR binding ISVD T017000975 (T017000700 with E1D mutation; SEQ ID NO: 34), two tumor anchoring ISVD building blocks directed against glypican-3 (GPC3) and a human serum albumin binding ISVD. The negative control (T017000698) did not comprise the anti-GPC3 ISVD building blocks.

### Impedance-Based Cytotoxicity Assay

ISVD constructs were characterized for redirected T cell mediated killing in an impedance-based cytotoxicity assay (e.g. as described in WO2018091606A1) using human or cynomolgus primary effector T cells and adherent target cells. Changes in impedance induced by the adherence of target cells to the surface of an electrode were measured using the xCELLigence instrument (Roche). T cells are non-adherent and therefore do not impact the impedance measurements. The xCELLigence® RTCA MP instrument quantifies the changes in electrical impedance, displaying them as a dimensionless parameter termed cell index, which is directly proportional to the total area of tissue-culture well that is covered by cells. To each well of a 96 E-plate (ACEA Biosciences; 05 232 368 001) 50 μL of 120 μM Alburex HSA (CSL Behring, 2160-679) was added to have a final concentration of 30 μM, in assay medium (target cell growth medium+1% penicillin/streptomycin (Life technologies Cat #15140)). Outer wells were not used and were filled with 200 μL medium or D-PBS. The 96 E-plate was placed in the xCELLigence® station (in the 37° C. incubator at 5% CO2) and a single measurement was performed to measure background impedance of the assay medium, in absence of cells. Subsequently, 50 μL target cells (2×1E4 cells/well) in assay medium were seeded onto the 96 E-plate, and 50 μL of serially diluted ISVD construct solutions (4× concentration) in assay medium was added. (Final volume=200 μL). After 30 min at room temperature, 50 μL of primary T 5 cells (3×1E5 cells/well) in assay medium were added per well to achieve an effector to target ratio of 15:1. The plate was placed in the xCELLigence® station and impedance was measured every 15 min for 4 days. The data was analyzed at a fixed time point (60 hours).

Generated constructs are shown below in Table 13.

TABLE 13
List of TCE-GPC3-GPC3-ALB and control constructs

| Construct ID | TCE ISVD ID | SEQ ID |
|---|---|---|
| A022600427 | T017000975 | 73 |
| A022600462 | T017000991 | 74 |
| T017000698 | T017000975 | 75 |

### Results

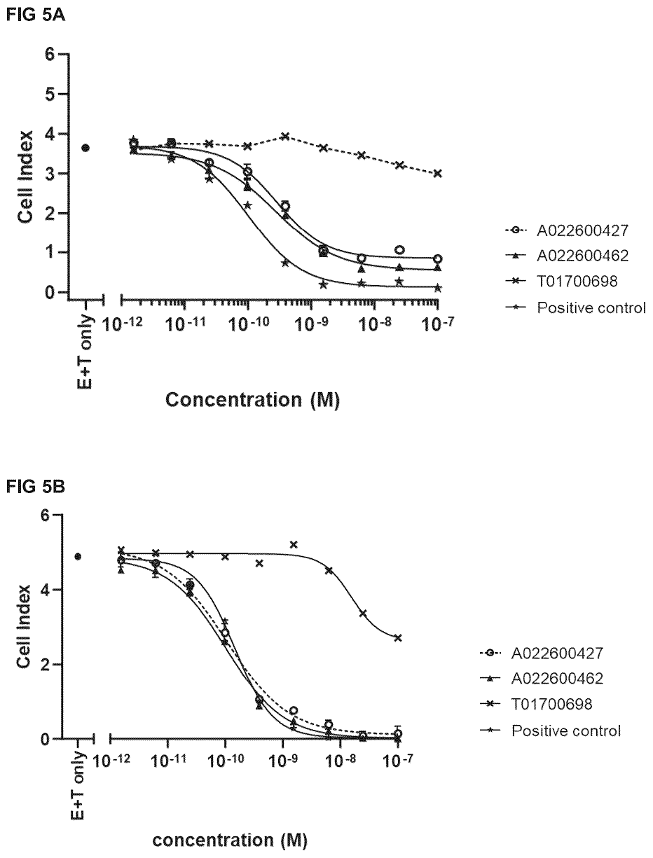

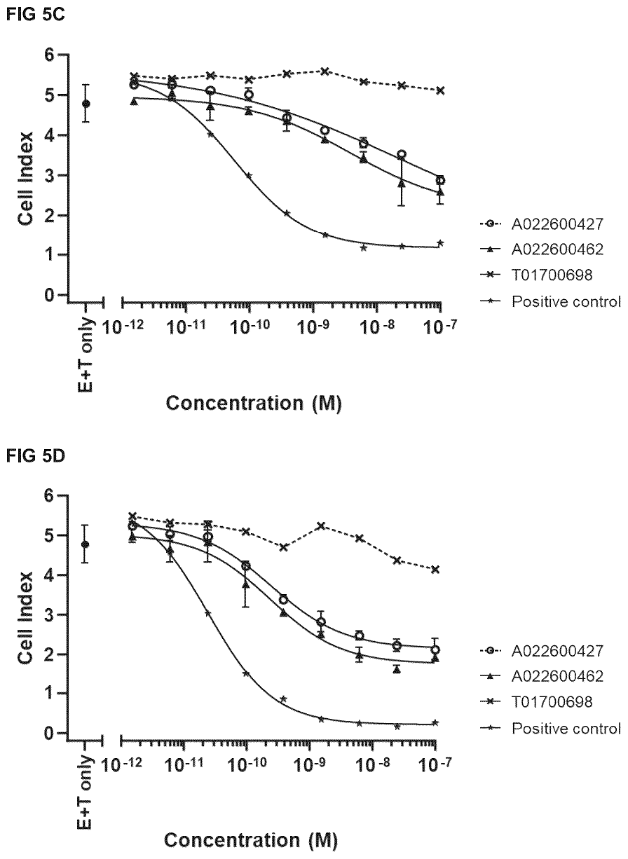

TCE ISVD variant T017000991 (SEQ ID NO: 42) was compared to the reference T017000975 (SEQ ID NO: 34) in a fusion construct with two ISVD building blocks against the tumor anchor GPC3 (Glypican-3) and an ISVD building block directed against Albumin linked by GS linkers. As a negative control, a construct without the GPC3 binding ISVDs was generated (T017000698, Table 13). As a positive control, a GPC3 binding bispecific antibody was generated. One arm of this antibody binds GPC3 and the other binds CD3. Graphical illustration of the results is shown in FIGS. 5A-5D. Calculated IC50 values for the target cell killing are shown in Table 14.

TABLE 14
IC50 (M) of the TCE-GPC3-GPC3-ALB constructs in the impedance-based
human and cynomolgus T cell mediated HuH-7 cell killing assay using
an effector to target ratio of 15 to 1 in the presence of 30 μM HSA

| | TCE-GPC3-GPC3-ALB mediated HuH-7 depletion | | | |
|---|---|---|---|---|
| | Human Donor | Human Donor | Cynomolgus | Cynomolgus |
| | #1 IC50 | #2 IC50 | Donor #1 IC50 | Donor #2 IC50 |
| Sample ID | (M) | (M) | (M) | (M) |
| A022600427 | 3.0E−10 | 1.1E−10 | 2.3E−08 | 2.5E−10 |
| A022600462 | 2.8E−10 | 1.0E−10 | 3.9E−09 | 2.2E−10 |
| T017000698 | negative | poor curve fit | negative | negative |
| Positive control | 1.0E−10 | 1.5E−10 | 6.0E−11 | 2.5E−11 |

The IC50 values for the two constructs with the TCE building blocks fused to the GPC3-binding ISVDs are comparable when using human or cynomolgus T cells. As expected, the construct without the GPC3-binding ISVDs, i.e. only the TCRαβ-binding ISVD fused to the human serum albumin-binding ISVD, did not mediate any, or only low, levels of cell killing.

### 6.11 Example 11: Chemical Stability Assessment of TCE ISVDs

To evaluate the chemical stability of the TCE ISVDs according to the present technology a forced degradation study was conducted with T017000991 (SEQ ID NO: 42) and compared to reference T017000975 (SEQ ID NO: 34) to both of which a HIS6-tag was attached (SEQ ID NO: 78).

The study consisted of a forced oxidation with 10 mM of H₂O₂ and a four-week incubation period at both 25° C. and 40° C., mimicking accelerated conditions and stress conditions respectively. After the stress test, the stability of T017000991 was analyzed by peptide mapping using trypsin digestion, a peptide separation by reverse phase chromatography and mass spectrometry MS/MS detection. The following modifications of the molecule were screened for: deamidation, isomerization/racemization and methionine or tryptophan oxidation. To evaluate the value of the sequence optimization of T017000991, the chemical stability of the ISVD with the chemical modifications was compared to reference T017000975.

### Results

The results of the peptide mapping analysis for the different temperature conditions are presented below in Table 15.

TABLE 15
Comparison of the Tryptic peptide map
analysis of T017000991 and T017000975
(control) after temperature stress.

| | | | |
|---|---|---|---|
| | | % Peptide on | % Peptide on |
| Seq | Modifiers | T017000975 | T017000991 |

| Fragment | | ID | (Kabat | | Δ 4w | Δ 4w | | Δ 4w | Δ 4w |
|---|---|---|---|---|---|---|---|---|---|
| label | Peptide | NO | numbering) | T0 | 25° C. | 40° C. | T0 | 25° C. | 40° C. |
| T7 | VAHISIGDQ | 182 | Isomerisation D61 | 1.0 | +0.3 | +2.1 | N.D. | N.D. | N.D. |
| | TDYADSAK | | | | | | | | |
| T11 | NTVYLQM | \|183 | Deamidation N82a | 1.1 | 0.0 | +0.4 | 1.1 | +0.1 | +0.2 |
| | NSLRPEDT | | | | | | | | |
| | AAYYCR | | | | | | | | |
| T11 | NTVYLQM | 183 | Deamidation N82a | 0.5 | +0.2 | +1.0 | 0.7 | +0.1 | ±0.7 |
| | NSLRPEDT | | | | | | | | |
| | AAYYCR | | | | | | | | |
| T11 | NTVYLQM | 183 | Oxidation M82 | 1.6 | +1.2 | +0.5 | 1.4 | −0.1 | +2.8 |
| | NSLRPEDT | | | | | | | | |
| | AAYYCR | | | | | | | | |
| T13 | IWPYDYW | 184 | Oxidation W99 | 0.6 | +0.2 | +0.3 | N.D. | N.D. | N.D. |
| | GQGTLVTV | | | | | | | | |
| | SSAAAHHH | | | | | | | | |
| | HHHH | | | | | | | | |

No significant changes were observed under forced oxidation conditions. The sequence coverage was around 85% for both molecules. In combination with AspN digestion 100% sequence coverage was obtained for both molecules. No additional modification was detected.

Two chemical liabilities were identified in T017000975: D61 is prone to isomerization, and W99 to oxidation (Kabat numbering). As mentioned before, these two sensitive amino acids were substituted in T017000991 (D61E, W99Y) resulting in the complete removal of these two liabilities in the TCE ISVD according to the present technology under the evaluated conditions.

Consequently, the chemical stability of the TCE ISVDs according to the present technology has been improved with regards to the reference TCE ISVD.

### 6.12 Example 12: Chemical Stability of TCE ISVD as Part of a Formatted ISVD Construct

To further demonstrate the improvement of the stability of the TCE ISVDs according to the present technology, the chemical stability of T017000991 as part of a formatted ISVD construct was analyzed.

A short-term storage study was conducted. T017000991 as well as T017000975 (as a control) were formatted with two anti-GPC3 ISVDs and an ISVD binding to human serum albumin. The constructs are presented below in Table 16.

TABLE 16

| ISVD construct ID | SEQ ID NO | Format |
|---|---|---|
| A022600424 | 76 | T017000975-GPC3-GPC3-ALB |
| A022600462 | 74 | T017000991-GPC3-GPC3-ALB |

Tryptophan oxidation and aspartic acid isomerization have been observed during storage when a formatted molecule was stored as a liquid with a suitable formulation buffer.

At the time it was found that the tryptophan oxidation rate was dependent on pH, temperature, ISVD concentration, transition metal, and polysorbate 20. Therefore, in order to accelerate any potential oxidation event, a stress study was conducted. The stress conditions consisted of an incubation at 40° C./75% relative humidity (RH) ±5% RH, plus the addition of 100 ppm of Fe(II), as an oxidant, for 3 months in plastic tubes.

For this study, both ISVD constructs were formulated to 1 mg/mL in a 25 mM Histidine-HCl, 8% (w/v) sucrose, 0.01% (w/v) pH 6.5 buffer and stability at 25° C./60% RH ±5% RH was also monitored over a period of 3 months.

The stability profile was monitored by reverse phase chromatography following the peaks with relative retention time (RRT)<1.0 as an indication of tryptophan oxidation and by peptide mapping following trypsin digestion and reverse phase LC-MS/MS.

### Results

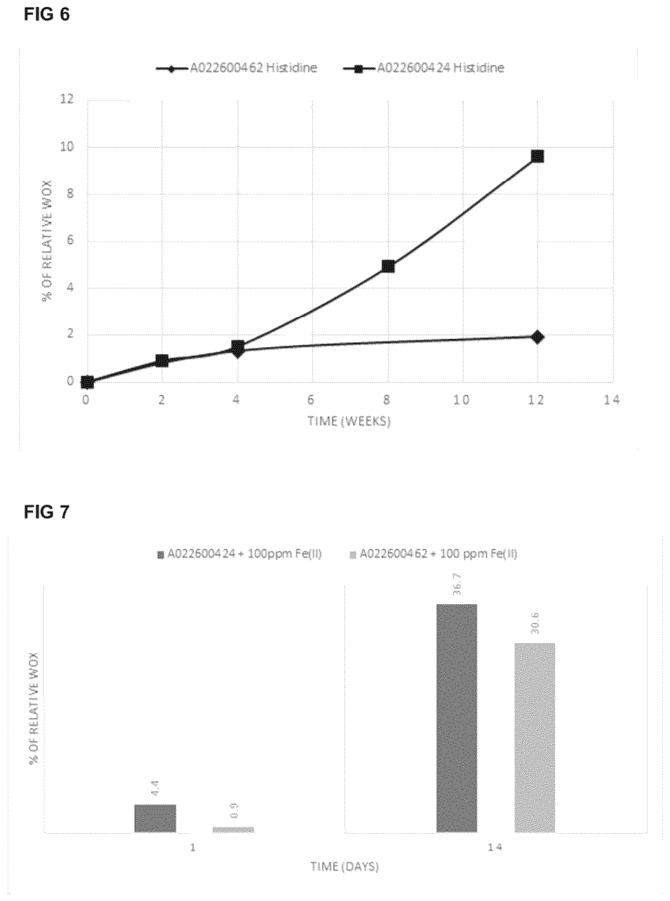

Graphical illustration of the results can be seen in FIG. 6, which shows the stability profiles at 25° C. by following the peak with RRT<1.0 by reverse phase chromatography. A reduction on the oxidation rate of the A022600462 was observed in comparison to A022600424, with a 5-fold decrease of tryptophan oxidation.

A similar trend can be observed during the evaluation of the stability profiles under stress conditions (FIG. 7). The addition of Fe(II) to the formulation solution catalyzed the oxidation rate of the ISVD constructs in presence of polysorbate 20 and Histidine. After one day of incubation at 40° C., the oxidation rate of A022600462 was around four-fold lower as compared to the control A022600424. The lower oxidation rate was still visible after 14 days of forced degradation conditions.

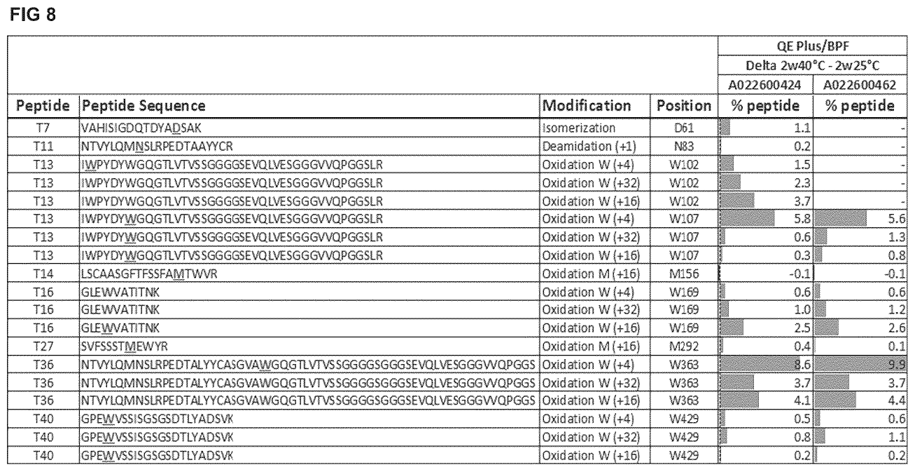

Peptide mapping was used to determine precisely where the different oxidation events were taking place in the amino acid sequence. The samples for temperature stress stability were analyzed, and compared, for this purpose. After trypsin digestion, almost 85% of the sequence can be covered by the produced peptides. Using the Biopharma Finder software (BPF; version 3.2) on the mass spectrometer (MS) the different peptides could be identified and chemical modifications on them elucidated. The results showed that there were different sites of Tryptophan oxidation on the selected formats, being the one on the TCE ISVD located in the peptide number 13, more precisely on W99 (A022600424) and W103 (A022600424 and A022600462). Results are shown in FIG. 8.

The peptide mapping analysis of the samples from the storage stability studies showed that the overall oxidation rate on A022600462 was much lower when compared to A0022600424, as the tryptophan W99 was removed from the former sequence. The oxidation rate on the second tryptophan seems to remain stable, while the tryptophan oxidation on other building blocks present in the format seems not to be impacted by the amino acid substitutions introduced. As expected, the removal of the D61 residue resulted in the removal of the isomerization liability in the final format.

In conclusion, the problems associated with tryptophan oxidation and isomerization in the ISVD have been overcome by substituting the amino acids in the relevant positions, while functionality of the ISVD has been maintained.

### 7 INDUSTRIAL APPLICABILITY

The ISVDs, polypeptides, nucleic acid molecules encoding the same, vectors comprising the nucleic acids and compositions described herein may be used for example in the treatment of subjects suffering from cancer.

TABLE A-6
Sequence listing

| | SEQ | |
|---|---|---|
| | ID | |
| Name | NO | Sequence |
| T017000700 | 1 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYWGQGTLVTVSS |
| T017000937 | 2 | EVQLVESGGGVVQPGGSLRLSCVASGYVHKINFYGWYRQAPGKERE |
| | | KVAHISIGDQTDYANSAKGRFTISRDESKNTVYLQMNSLRPEDTAAY |
| | | YCRALSRIWPYDYWGQGTLVTVSS |
| T017000938 | 3 | EVQLVESGGGVVQPGGSLRLSCVASGYVHKINFYGWYRQAPGKERE |
| | | KVAHISIGDQTDYAPSAKGRFTISRDESKNTVYLQMNSLRPEDTAAYY |
| | | CRALSRIWPYDYWGQGTLVTVSS |
| T017000939 | 4 | EVQLVESGGGVVQPGGSLRLSCVASGYVHKINFYGWYRQAPGKERE |
| | | KVAHISIGDQTDYAKSAKGRFTISRDESKNTVYLQMNSLRPEDTAAYY |
| | | CRALSRIWPYDYWGQGTLVTVSS |
| T017000940 | 5 | EVQLVESGGGVVQPGGSLRLSCVASGYVHKINFYGWYRQAPGKERE |
| | | KVAHISIGDQTDYARSAKGRFTISRDESKNTVYLQMNSLRPEDTAAYY |
| | | CRALSRIWPYDYWGQGTLVTVSS |
| T017000941 | 6 | EVQLVESGGGVVQPGGSLRLSCVASGYVHKINFYGWYRQAPGKERE |
| | | KVAHISIGDQTDYAISAKGRFTISRDESKNTVYLQMNSLRPEDTAAYY |
| | | CRALSRIWPYDYWGQGTLVTVSS |
| T017000942 | 7 | EVQLVESGGGVVQPGGSLRLSCVASGYVHKINFYGWYRQAPGKERE |
| | | KVAHISIGDQTDYATSAKGRFTISRDESKNTVYLQMNSLRPEDTAAYY |
| | | CRALSRIWPYDYWGQGTLVTVSS |
| T017000943 | 8 | EVQLVESGGGVVQPGGSLRLSCVASGYVHKINFYGWYRQAPGKERE |
| | | KVAHISIGDQTDYAHSAKGRFTISRDESKNTVYLQMNSLRPEDTAAYY |
| | | CRALSRIWPYDYWGQGTLVTVSS |
| T017000944 | 9 | EVQLVESGGGVVQPGGSLRLSCVASGYVHKINFYGWYRQAPGKERE |
| | | KVAHISIGDQTDYAVSAKGRFTISRDESKNTVYLQMNSLRPEDTAAYY |
| | | CRALSRIWPYDYWGQGTLVTVSS |
| T017000945 | 10 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIWPYDYWGQGTLVTVSS |
| T017000946 | 11 | EVQLVESGGGVVQPGGSLRLSCVASGYVHKINFYGWYRQAPGKERE |
| | | KVAHISIGDQTDYAASAKGRFTISRDESKNTVYLQMNSLRPEDTAAYY |
| | | CRALSRIWPYDYWGQGTLVTVSS |
| T017000947 | 12 | EVQLVESGGGVVQPGGSLRLSCVASGYVHKINFYGWYRQAPGKERE |
| | | KVAHISIGDQTDYAYSAKGRFTISRDESKNTVYLQMNSLRPEDTAAYY |
| | | CRALSRIWPYDYWGQGTLVTVSS |
| T017000948 | 13 | EVQLVESGGGVVQPGGSLRLSCVASGYVHKINFYGWYRQAPGKERE |
| | | KVAHISIGDQTDYALSAKGRFTISRDESKNTVYLQMNSLRPEDTAAYY |
| | | CRALSRIWPYDYWGQGTLVTVSS |
| T017000949 | 14 | EVQLVESGGGVVQPGGSLRLSCVASGYVHKINFYGWYRQAPGKERE |
| | | KVAHISIGDQTDYAQSAKGRFTISRDESKNTVYLQMNSLRPEDTAAY |
| | | YCRALSRIWPYDYWGQGTLVTVSS |
| T017000950 | 15 | EVQLVESGGGVVQPGGSLRLSCVASGYVHKINFYGWYRQAPGKERE |
| | | KVAHISIGDQTDYAFSAKGRFTISRDESKNTVYLQMNSLRPEDTAAYY |
| | | CRALSRIWPYDYWGQGTLVTVSS |
| T017000951 | 16 | EVQLVESGGGVVQPGGSLRLSCVASGYVHKINFYGWYRQAPGKERE |
| | | KVAHISIGDQTDYASSAKGRFTISRDESKNTVYLQMNSLRPEDTAAYY |
| | | CRALSRIWPYDYWGQGTLVTVSS |
| T017000952 | 17 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYRGQGTLVTVSS |
| T017000953 | 18 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYAGQGTLVTVSS |
| T017000954 | 19 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYEGQGTLVTVSS |
| T017000955 | 20 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYYGQGTLVTVSS |
| T017000956 | 21 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYLGQGTLVTVSS |
| T017000957 | 22 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYHGQGTLVTVSS |
| T017000958 | 23 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYIGQGTLVTVSS |
| T017000959 | 24 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYQGQGTLVTVSS |
| T017000960 | 25 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYVGQGTLVTVSS |
| T017000961 | 26 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYKGQGTLVTVSS |
| T017000962 | 27 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYSGQGTLVTVSS |
| T017000963 | 28 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYGGQGTLVTVSS |
| T017000964 | 29 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYPGQGTLVTVSS |
| T017000965 | 30 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYFGQGTLVTVSS |
| T017000966 | 31 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYTGQGTLVTVSS |
| T017000967 | 32 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIYPYDYWGQGTLVTVSS |
| T017000974 | 33 | DVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYWGQGTLVTVSS |
| T017000975 | 34 | EVQLVESGGGVVQPGGSLRLSCVASGYVHKINFYGWYRQAPGKERE |
| | | KVAHISIGDQTDYAPSAKGRFTISRDESKNTVYLQMNSLRPEDTAAYY |
| | | CRALSRIYPYDYWGQGTLVTVSS |
| T017000976 | 35 | EVQLVESGGGVVQPGGSLRLSCVASGYVHKINFYGWYRQAPGKERE |
| | | KVAHISIGDQTDYARSAKGRFTISRDESKNTVYLQMNSLRPEDTAAYY |
| | | CRALSRIYPYDYWGQGTLVTVSS |
| T017000977 | 36 | EVQLVESGGGVVQPGGSLRLSCVASGYVHKINFYGWYRQAPGKERE |
| | | KVAHISIGDQTDYAVSAKGRFTISRDESKNTVYLQMNSLRPEDTAAYY |
| | | CRALSRIYPYDYWGQGTLVTVSS |
| T017000978 | 37 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIYPYDYWGQGTLVTVSS |
| T017000979 | 38 | EVQLVESGGGVVQPGGSLRLSCVASGYVHKINFYGWYRQAPGKERE |
| | | KVAHISIGDQTDYAASAKGRFTISRDESKNTVYLQMNSLRPEDTAAYY |
| | | CRALSRIYPYDYWGQGTLVTVSS |
| T017000980 | 39 | EVQLVESGGGVVQPGGSLRLSCVASGYVHKINFYGWYRQAPGKERE |
| | | KVAHISIGDQTDYAQSAKGRFTISRDESKNTVYLQMNSLRPEDTAAY |
| | | YCRALSRIYPYDYWGQGTLVTVSS |
| T017000981 | 40 | EVQLVESGGGVVQPGGSLRLSCVASGYVHKINFYGWYRQAPGKERE |
| | | KVAHISIGDQTDYASSAKGRFTISRDESKNTVYLQMNSLRPEDTAAYY |
| | | CRALSRIYPYDYWGQGTLVTVSS |
| T017000982 | 41 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIYPYDYYGQGTLVTVSS |
| T017000991 | 42 | DVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIYPYDYWGQGTLVTVSS |
| T017000992 | 43 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIAPYDYWGQGTLVTVSS |
| T017000993 | 44 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIPPYDYWGQGTLVTVSS |
| T017000994 | 45 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIDPYDYWGQGTLVTVSS |
| T017000995 | 46 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIQPYDYWGQGTLVTVSS |
| T017000996 | 47 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIEPYDYWGQGTLVTVSS |
| T017000997 | 48 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIRPYDYWGQGTLVTVSS |
| T017000998 | 49 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIFPYDYWGQGTLVTVSS |
| T017000999 | 50 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRISPYDYWGQGTLVTVSS |
| T017001000 | 51 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIGPYDYWGQGTLVTVSS |
| T017001001 | 52 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRITPYDYWGQGTLVTVSS |
| T017001002 | 53 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIHPYDYWGQGTLVTVSS |
| T017001003 | 54 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIVPYDYWGQGTLVTVSS |
| T017001004 | 55 | EVQLVESGGGVVQPGGSLRLSCVASGYVHKINFYGWYRQAPGKERE |
| | | KVAHISIGDQTDYASAKGRFTISRDESKNTVYLQMNSLRPEDTAAYYC |
| | | RALSRIKPYDYWGQGTLVTVSS |
| T017001005 | 56 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRILPYDYWGQGTLVTVSS |
| T017001006 | 57 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIIPYDYWGQGTLVTVSS |
| T017001017 | 58 | EVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYWGQGTLVTVSSGGGGSGGGSEVQLVESGGGVVQ |
| | | PGGSLRLSCAASGTTLDYYVIGWFRQAPGKEREGVSCISSSDGYTYYA |
| | | DSVKGRFTISRDNAKNTVYLQMNSLRPEDTALYYCATDPYYSSCHPFF |
| | | ADYEYDYWGQGTLVTVSSGGGGSGGGSEVQLVESGGGVVQPGGSL |
| | | RLSCAASGFTFRSFGMSWVRQAPGKGPEWVSSISGSGSDTLYADSV |
| | | KGRFTISRDNSKNTLYLQMNSLRPEDTALYYCTIGGSLSRSSQGTLVT |
| | | VSS |
| T017001018 | 59 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIYPYDYWGQGTLVTVSSGGGGSGGGSEVQLVESGGGVVQP |
| | | GGSLRLSCAASGTTLDYYVIGWFRQAPGKEREGVSCISSSDGYTYYAD |
| | | SVKGRFTISRDNAKNTVYLQMNSLRPEDTALYYCATDPYYSSCHPFFA |
| | | DYEYDYWGQGTLVTVSSGGGGSGGGSEVQLVESGGGVVQPGGSLR |
| | | LSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVS |
| | | S |
| T017001019 | 60 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRITPYDYWGQGTLVTVSSGGGGSGGGSEVQLVESGGGVVQP |
| | | GGSLRLSCAASGTTLDYYVIGWFRQAPGKEREGVSCISSSDGYTYYAD |
| | | SVKGRFTISRDNAKNTVYLQMNSLRPEDTALYYCATDPYYSSCHPFFA |
| | | DYEYDYWGQGTLVTVSSGGGGSGGGSEVQLVESGGGVVQPGGSLR |
| | | LSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVS |
| | | S |
| T017001020 | 61 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRISPYDYWGQGTLVTVSSGGGGSGGGSEVQLVESGGGVVQP |
| | | GGSLRLSCAASGTTLDYYVIGWFRQAPGKEREGVSCISSSDGYTYYAD |
| | | SVKGRFTISRDNAKNTVYLQMNSLRPEDTALYYCATDPYYSSCHPFFA |
| | | DYEYDYWGQGTLVTVSSGGGGSGGGSEVQLVESGGGVVQPGGSLR |
| | | LSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVS |
| | | S |
| T017001021 | 62 | EVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIQPYDYWGQGTLVTVSSGGGGSGGGSEVQLVESGGGVVQ |
| | | PGGSLRLSCAASGTTLDYYVIGWFRQAPGKEREGVSCISSSDGYTYYA |
| | | DSVKGRFTISRDNAKNTVYLQMNSLRPEDTALYYCATDPYYSSCHPFF |
| | | ADYEYDYWGQGTLVTVSSGGGGSGGGSEVQLVESGGGVVQPGGSL |
| | | RLSCAASGFTFRSFGMSWVRQAPGKGPEWVSSISGSGSDTLYADSV |
| | | KGRFTISRDNSKNTLYLQMNSLRPEDTALYYCTIGGSLSRSSQGTLVT |
| | | VSS |
| T017001022 | 63 | DVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYWGQGTLVTVSSGGGGSGGGSEVQLVESGGGVVQ |
| | | PGGSLRLSCAASGTTLDYYVIGWFRQAPGKEREGVSCISSSDGYTYYA |
| | | DSVKGRFTISRDNAKNTVYLQMNSLRPEDTALYYCATDPYYSSCHPFF |
| | | ADYEYDYWGQGTLVTVSSGGGGSGGGSEVQLVESGGGVVQPGGSL |
| | | RLSCAASGFTFRSFGMSWVRQAPGKGPEWVSSISGSGSDTLYADSV |
| | | KGRFTISRDNSKNTLYLQMNSLRPEDTALYYCTIGGSLSRSSQGTLVT |
| | | VSS |
| T017001023 | 64 | DVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIYPYDYWGQGTLVTVSSGGGGSGGGSEVQLVESGGGVVQP |
| | | GGSLRLSCAASGTTLDYYVIGWFRQAPGKEREGVSCISSSDGYTYYAD |
| | | SVKGRFTISRDNAKNTVYLQMNSLRPEDTALYYCATDPYYSSCHPFFA |
| | | DYEYDYWGQGTLVTVSSGGGGSGGGSEVQLVESGGGVVQPGGSLR |
| | | LSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVS |
| | | S |
| T017001024 | 65 | DVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRITPYDYWGQGTLVTVSSGGGGSGGGSEVQLVESGGGVVQP |
| | | GGSLRLSCAASGTTLDYYVIGWFRQAPGKEREGVSCISSSDGYTYYAD |
| | | SVKGRFTISRDNAKNTVYLQMNSLRPEDTALYYCATDPYYSSCHPFFA |
| | | DYEYDYWGQGTLVTVSSGGGGSGGGSEVQLVESGGGVVQPGGSLR |
| | | LSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVS |
| | | S |
| T017001025 | 66 | DVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRISPYDYWGQGTLVTVSSGGGGSGGGSEVQLVESGGGVVQP |
| | | GGSLRLSCAASGTTLDYYVIGWFRQAPGKEREGVSCISSSDGYTYYAD |
| | | SVKGRFTISRDNAKNTVYLQMNSLRPEDTALYYCATDPYYSSCHPFFA |
| | | DYEYDYWGQGTLVTVSSGGGGSGGGSEVQLVESGGGVVQPGGSLR |
| | | LSCAASGFTFRSFGMSWVRQAPGKGPEWVSSISGSGSDTLYADSVK |
| | | GRFTISRDNSKNTLYLQMNSLRPEDTALYYCTIGGSLSRSSQGTLVTVS |
| | | S |
| T017001026 | 67 | DVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIQPYDYWGQGTLVTVSSGGGGSGGGSEVQLVESGGGVVQ |
| | | PGGSLRLSCAASGTTLDYYVIGWFRQAPGKEREGVSCISSSDGYTYYA |
| | | DSVKGRFTISRDNAKNTVYLQMNSLRPEDTALYYCATDPYYSSCHPFF |
| | | ADYEYDYWGQGTLVTVSSGGGGSGGGSEVQLVESGGGVVQPGGSL |
| | | RLSCAASGFTFRSFGMSWVRQAPGKGPEWVSSISGSGSDTLYADSV |
| | | KGRFTISRDNSKNTLYLQMNSLRPEDTALYYCTIGGSLSRSSQGTLVT |
| | | VSS |
| T017001027 | 68 | EVQLVESGGGVVQPGGSLRLSCAASGTTLDYYVIGWFRQAPGKERE |
| | | GVSCISSSDGYTYYADSVKGRFTISRDNAKNTVYLQMNSLRPEDTALY |
| | | YCATDPYYSSCHPFFADYEYDYWGQGTLVTVSSGGGGSGGGSEVQL |
| | | VESGGGVVQPGGSLRLSCVASGYVHKINFYGWYRQAPGKEREKVAH |
| | | ISIGDQTDYADSAKGRFTISRDESKNTVYLQMNSLRPEDTAAYYCRAL |
| | | SRIWPYDYWGQGTLVTVSSGGGGSGGGSEVQLVESGGGVVQPGG |
| | | SLRLSCAASGFTFRSFGMSWVRQAPGKGPEWVSSISGSGSDTLYADS |
| | | VKGRFTISRDNSKNTLYLQMNSLRPEDTALYYCTIGGSLSRSSQGTLV |
| | | TVSS |
| T017001028 | 69 | EVQLVESGGGVVQPGGSLRLSCAASGTTLDYYVIGWFRQAPGKERE |
| | | GVSCISSSDGYTYYADSVKGRFTISRDNAKNTVYLQMNSLRPEDTALY |
| | | YCATDPYYSSCHPFFADYEYDYWGQGTLVTVSSGGGGSGGGSEVQL |
| | | VESGGGVVQPGGSLRLSCVASGYVHKINFYGWYRQAPGKEREKVAH |
| | | ISIGDQTDYAESAKGRFTISRDESKNTVYLQMNSLRPEDTAAYYCRAL |
| | | SRIYPYDYWGQGTLVTVSSGGGGSGGGSEVQLVESGGGVVQPGGS |
| | | LRLSCAASGFTFRSFGMSWVRQAPGKGPEWVSSISGSGSDTLYADS |
| | | VKGRFTISRDNSKNTLYLQMNSLRPEDTALYYCTIGGSLSRSSQGTLV |
| | | TVSS |
| T017001029 | 70 | EVQLVESGGGVVQPGGSLRLSCAASGTTLDYYVIGWFRQAPGKERE |
| | | GVSCISSSDGYTYYADSVKGRFTISRDNAKNTVYLQMNSLRPEDTALY |
| | | YCATDPYYSSCHPFFADYEYDYWGQGTLVTVSSGGGGSGGGSEVQL |
| | | VESGGGVVQPGGSLRLSCVASGYVHKINFYGWYRQAPGKEREKVAH |
| | | ISIGDQTDYAESAKGRFTISRDESKNTVYLQMNSLRPEDTAAYYCRAL |
| | | SRITPYDYWGQGTLVTVSSGGGGSGGGSEVQLVESGGGVVQPGGS |
| | | LRLSCAASGFTFRSFGMSWVRQAPGKGPEWVSSISGSGSDTLYADS |
| | | VKGRFTISRDNSKNTLYLQMNSLRPEDTALYYCTIGGSLSRSSQGTLV |
| | | TVSS |
| T017001030 | 71 | EVQLVESGGGVVQPGGSLRLSCAASGTTLDYYVIGWFRQAPGKERE |
| | | GVSCISSSDGYTYYADSVKGRFTISRDNAKNTVYLQMNSLRPEDTALY |
| | | YCATDPYYSSCHPFFADYEYDYWGQGTLVTVSSGGGGSGGGSEVQL |
| | | VESGGGVVQPGGSLRLSCVASGYVHKINFYGWYRQAPGKEREKVAH |
| | | ISIGDQTDYAESAKGRFTISRDESKNTVYLQMNSLRPEDTAAYYCRAL |
| | | SRISPYDYWGQGTLVTVSSGGGGSGGGSEVQLVESGGGVVQPGGS |
| | | LRLSCAASGFTFRSFGMSWVRQAPGKGPEWVSSISGSGSDTLYADS |
| | | VKGRFTISRDNSKNTLYLQMNSLRPEDTALYYCTIGGSLSRSSQGTLV |
| | | TVSS |
| T017001031 | 72 | EVQLVESGGGVVQPGGSLRLSCAASGTTLDYYVIGWFRQAPGKERE |
| | | GVSCISSSDGYTYYADSVKGRFTISRDNAKNTVYLQMNSLRPEDTALY |
| | | YCATDPYYSSCHPFFADYEYDYWGQGTLVTVSSGGGGSGGGSEVQL |
| | | VESGGGVVQPGGSLRLSCVASGYVHKINFYGWYRQAPGKEREKVAH |
| | | ISIGDQTDYAESAKGRFTISRDESKNTVYLQMNSLRPEDTAAYYCRAL |
| | | SRIQPYDYWGQGTLVTVSSGGGGSGGGSEVQLVESGGGVVQPGGS |
| | | LRLSCAASGFTFRSFGMSWVRQAPGKGPEWVSSISGSGSDTLYADS |
| | | VKGRFTISRDNSKNTLYLQMNSLRPEDTALYYCTIGGSLSRSSQGTLV |
| | | TVSS |
| A022600427 | 73 | DVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYWGQGTLVTVSSGGGGSEVQLVESGGGVVQPGGS |
| | | LRLSCAASGFTFSSFAMTWVRRPPGKGLEWVATITNAGVTSYADSV |
| | | KGRFTISRDNAKNTLYLQMNSLRPEDTALYICANARRTGPRAPTDIGS |
| | | YRGQGTLVTVSSGGGGSGGGSEVQLVESGGGVVQPGGSLRLSCAAS |
| | | GSIFRSVFSSSTMEWYRQAPGKKRELVARIAPGEGTYYGALYADSVK |
| | | GRFTISRDNAKNTVYLQMNSLRPEDTALYYCASGVAWGQGTLVTVS |
| | | SGGGGSGGGSEVQLVESGGGVVQPGGSLRLSCAASGFTFRSFGMS |
| | | WVRQAPGKGPEWVSSISGSGSDTLYADSVKGRFTISRDNSKNTLYLQ |
| | | MNSLRPEDTALYYCTIGGSLSRSSQGTLVTVSSA |
| A022600462 | 74 | DVQLVESGGGVVQPGGSLRLSCVASGYVHKINFYGWYRQAPGKERE |
| | | KVAHISIGDQTDYAESAKGRFTISRDESKNTVYLQMNSLRPEDTAAYY |
| | | CRALSRIYPYDYWGQGTLVTVSSGGGGSEVQLVESGGGVVQPGGSL |
| | | RLSCAASGFTFSSFAMTWVRRPPGKGLEWVATITNKGVTSYADSVK |
| | | GRFTISRDNAKNTLYLQMNSLRPEDTALYICANARRTGPRAPTDIGSY |
| | | RGQGTLVTVSSGGGGSGGGSEVQLVESGGGVVQPGGSLRLSCAAS |
| | | GSIFRSVFSSSTMEWYRQAPGKKRELVARIAPGEGTYYGALYADSVK |
| | | GRFTISRDNAKNTVYLQMNSLRPEDTALYYCASGVAWGQGTLVTVS |
| | | SGGGGSGGGSEVQLVESGGGVVQPGGSLRLSCAASGFTFRSFGMS |
| | | WVRQAPGKGPEWVSSISGSGSDTLYADSVKGRFTISRDNSKNTLYLQ |
| | | MNSLRPEDTALYYCTIGGSLSRSSQGTLVTVSSA |
| T017000698 | 75 | DVQLVESGGGVVQPGGSLRLSCVASGYVHKINFYGWYRQAPGKERE |
| | | KVAHISIGDQTDYADSAKGRFTISRDESKNTVYLQMNSLRPEDTAAYY |
| | | CRALSRIWPYDYWGQGTLVTVSSGGGGSGGGSEVQLVESGGGVVQ |
| | | PGGSLRLSCAASGFTFRSFGMSWVRQAPGKGPEWVSSISGSGSDTL |
| | | YADSVKGRFTISRDNSKNTLYLQMNSLRPEDTALYYCTIGGSLSRSSQ |
| | | GTLVTVSSA |
| A022600424 | 76 | VQLVESGGGVVQPGGSLRLSCVASGYVHKINFYGWYRQAPGKEREK |
| | | VAHISIGDQTDYADSAKGRFTISRDESKNTVYLQMNSLRPEDTAAYYC |
| | | RALSRIWPYDYWGQGTLVTVSSGGGGSEVQLVESGGGVVQPGGSL |
| | | RLSCAASGFTFSSFAMTWVRRPPGKGLEWVATITNKGVTSYADSVK |
| | | GRFTISRDNAKNTLYLQMNSLRPEDTALYICANARRTGPRAPTDIGSY |
| | | RGQGTLVTVSSGGGGSGGGSEVQLVESGGGVVQPGGSLRLSCAAS |
| | | GSIFRSVFSSSTMEWYRQAPGKKRELVARIAPGEGTYYGALYADSVK |
| | | GRFTISRDNAKNTVYLQMNSLRPEDTALYYCASGVAWGQGTLVTVS |
| | | SGGGGSGGGSEVQLVESGGGVVQPGGSLRLSCAASGFTFRSFGMS |
| | | WVRQAPGKGPEWVSSISGSGSDTLYADSVKGRFTISRDNSKNTLYLQ |
| | | MNSLRPEDTALYYCTIGGSLSRSSQGTLVTVSSA |
| FLAG3-HIS6 tag | 77 | AAADYKDHDGDYKDHDIDYKDDDDKGAAHHHHHH |
| HIS6 tag | 78 | HHHHHH |
| CDR1 of TCE | 79 | INFYG |
| ISVD (Kabat) | | |
| CDR2 of TCE | 80 | HISIGDQTDYAX₁SAKG |
| ISVD (Kabat) | | |
| CD3 of TCE ISVD | 81 | LSRIX₂PYDY |
| (Kabat) | | |
| CDR1 of TCE | 82 | GYVHKINFYG |
| ISVD (Abm) | | |
| CDR2 of TCE | 83 | HISIGDQTD |
| ISVD (Abm) | | |
| CDR3 of TCE | 84 | LSRIX₂PYDY |
| ISVD (Abm) | | |
| TCE ISVD | 85 | X₀QVQLVESGGGVVQPGGSLRLSCVASGYVHKINFYGWYRQAPGKER |
| | | EKVAHISIGDQTDYAX₁SAKGRFTISRDESKNTVYLQMNSLRPEDTAA |
| | | YYCRALSRIX₂PYDYX₃GQGTLVTVSS |
| 5GS linker | 86 | GGGGS |
| 9GS linker | 87 | GGGGSGGGS |
| C-terminus ISVD | 88 | VTVSS |
| C-terminus ISVD | 89 | VKVSS |
| C-terminus ISVD | 90 | VQVSS |
| C-terminus ISVD | 91 | VTVKS |
| C-terminus ISVD | 92 | VTVQS |
| C-terminus ISVD | 93 | VKVKS |
| C-terminus ISVD | 94 | VKVQS |
| C-terminus ISVD | 95 | VQVKS |
| C-terminus ISVD | 96 | VQVQS |
| C-terminus ISVD | 97 | VTVSSA |
| C-terminus ISVD | 98 | VKVSSA |
| C-terminus ISVD | 99 | VQVSSA |
| C-terminus ISVD | 100 | VTVKSA |
| C-terminus ISVD | 101 | VTVQSA |
| C-terminus ISVD | 102 | VKVKSA |
| C-terminus ISVD | 103 | VKVQSA |
| C-terminus ISVD | 104 | VQVKSA |
| C-terminus ISVD | 105 | VQVQSA |
| Human TCR | 106 | PNIQNPDPAVYQLRDSKSSDKSVCLFTDFDSQTNVSQSKDSDVYITD |
| alpha constant | | KTVLDMRSMDFKSNSAVAWSNKSDFACANAFNNSIIPEDTFFPSPES |
| domain (derived | | SCDVKLVEKSFETDTNLNFQNLSVIGFRILLLKVAGFNLLMTLRLWSS |
| from P01848) | | |
| beta constant | 107 | EDLNKVFPPEVAVFEPSEAEISHTQKATLVCLATGFFPDHVELSWWV |
| domain (derived | | NGKEVHSGVSTDPQPLKEQPALNDSRYALSSRLRVSATFWQNPRNH |
| from P01850) | | FRCQVQFYGLSENDEWTQDRAKPVTQIVSAEAWGRADCGFTSVSY |
| | | QQGVLSATILYEILLGKATLYAVLVSALVLMAMVKRKDF |
| Rhesus TCR | 108 | MLLLLVLVLEVIFTLGGTRAQSVTQLDSQVSVSEGVPVLLRCNYSSSFS |
| alpha | | PYLFWYVQYPNQGLQLLLKYTSGTTLVKGINGFEAEFKKSETSFHLTK |
| (AEA41863) | | ASAHVSDAAEYFCALARGALVFGKGTRLSVIPNIQNPDPAVYQLRGS |
| | | KSNDTSVCLFTDFDSVMNVSQSKDSDVHITDKTVLDMRSMDFKSN |
| | | GAVAWSNKSDFACTSAFKDSVIPADTFFPGTESVCDANLVEKSFETD |
| | | MNLNFQNLSVIGFRILLLKVAGFNLLMTLRLWSS |
| Rhesus TCR beta | 109 | MGFWTLCCVSFCILVAKHTDAGVIQLPRHEVTEMGKEVTLRCEPISG |
| (AEA41864) | | HSSLFWYRQTMMRGLEFLIYFNNKSPIDDSGMPKDRFSATMPDASF |
| | | STLKIQPSEPRDSAVYFCASTPGQGREKLFFGSGTQLSVLEDLKKVFPP |
| | | KVAVFEPSEAEISHTQKATLVCLATGFYPDHVELSWWVNGKEVHSG |
| | | VSTDPQPLKEQPALEDSRYCLSSRLRVSATFWHNPRNHFRCQVQFY |
| | | GLSEDDEWTEDRDKPITQKISAEVWGRADCGFTSVSYQQGVLSATIL |
| | | YEILLGKATLYAVLVSALMLMAMVKRKDF |
| Human TCR- | 110 | MNMRPVTSSVLVLLLMLRRSNGQLLEQSPQFLSIQEGENLTVYCNSS |
| zipper: alpha | | SVFSSLQWYRQEPGEGPVLLVTVVTGGEVKKLKRLTFQFGDARKDSS |
| chain | | LHITAAQPGDTGLYLCAGAGSQGNLIFGKGTKLSVKPNIQNPDPAVY |
| | | QLRDSKSSDKSVCLFTDFDSQTNVSQSKDSDVYITDKSVLDMRSMDF |
| | | KSNSAVAWSNKSDFACANAFNNSIIPEDTFFPSPESSCSSADLVPRGS |
| | | TTAPSAQLKKKLQALKKKNAQLKWKLQALKKKLAQEQKLISEEDL |
| Human TCR- | 111 | MSNTVLADSAWGITLLSWVTVFLLGTSSADGGITQSPKYLFRKEGQN |
| zipper: beta | | VTLSCEQNLNHDAMYWYRQDPGQGLRLIYYSQIVNDFQKGDIAEGY |
| chain | | SVSREKKESFPLTVTSAQKNPTAFYLCASSSRSSYEQYFGPGTRLTVTE |
| | | DLKNVFPPEVAVFEPSEAEISHTQKATLVCLATGFYPDHVELSWWVN |
| | | GKEVHSGVSTDPQPLKEQPALNDSRYSLSSRLRVSATFWQNPRNHF |
| | | RCQVQFYGLSENDEWTQDRAKPVTQIVSAEAWGRADCSSADLVPR |
| | | GSTTAPSAQLEKELQALEKENAQLEWELQALEKELAQTGHHHHHHH |
| | | HHH |
| Cyno TCR- | 112 | MLLITSVLVLWMQLSQVNGQQIMQIPQYQHVQEGEDFTTYCNSST |
| zipper: alpha | | TLSNIQWYKQRPGGHPVFLIMLVKSGEVKKQKRLIFQFGEAKKNSSL |
| chain (derived | | HITATQTTDVGTYFCATTGVNNLFFGTGTRLTVLPYIQNPDPAVYQLR |
| from AEA41865) | | GSKSNDTSVCLFTDFDSVMNVSQSKDSDVHITDKTVLDMRSMDFKS |
| | | NGAVAWSNKSDFACTSAFKDSVIPADTFFPSPESSCSSADLVPRGSTT |
| | | APSAQLKKKLQALKKKNAQLKWKLQALKKKLAQEQKLISEEDL |
| Cyno TCR- | 113 | MSIGLLCCAALSLLWAGPVNAGVTQTPKFQVLKTGQSMTLQCAQD |
| zipper: beta | | MNHDYMYWYRQDPGMGLRLIHYSVGEGSTEKGEVPDGYNVTRSN |
| chain (derived | | TEDFPLRLESAAPSQTSVYFCASSYWTGRSYEQYFGPGTRLTVIEDLKK |
| from AEA41866 | | VFPPKVAVFEPSEAEISHTQKATLVCLATGFYPDHVELSWWVNGKEV |
| and AEA41868) | | HSGVSTDPQPLKEQPALEDSRYSLSSRLRVSATFWHNPRNHFRCQV |
| | | QFYGLSEDDEWTEDRDKPITQKISAEAWGRADCSSADLVPRGSTTAP |
| | | SAQLEKELQALEKENAQLEWELQALEKELAQTGHHHHHHHHHH |
| Human TCR | 114 | PNIQNPDPAVYQLRDSKSSDKSVCLFTDFDSQTNVSQSKDSDVYITD |
| alpha constant | | KTVLDMRSMDFKSNSAVAWSNKSDFACANAFNNSIIPEDTFFPSPES |
| domain (derived | | SC |
| from P01848) | | |
| Human TCR | 115 | EDLNKVFPPEVAVFEPSEAEISHTQKATLVCLATGFFPDHVELSWWV |
| beta constant | | NGKEVHSGVSTDPQPLKEQPALNDSRYALSSRLRVSATFWQNPRNH |
| domain (derived | | FRCQVQFYGLSENDEWTQDRAKPVTQIVSAEAWGRADC |
| from P01850) | | |
| cynomolgus | 116 | PYIQNPDPAVYQLRGSKSNDTSVCLFTDFDSVMNVSQSKDSDVHITD |
| TCR-alpha | | KTVLDMRSMDFKSNGAVAWSNKSDFACTSAFKDSVIPADTFFPSPE |
| constant | | SSC |
| domain (derived | | |
| from AEA41865) | | |
| cynomolgus | 117 | EDLKKVFPPKVAVFEPSEAEISHTQKATLVCLATGFYPDHVELSWWV |
| TCR-beta | | NGKEVHSGVSTDPQPLKEQPALEDSRYSLSSRLRVSATFWHNPRNHF |
| constant | | RCQVQFYGLSEDDEWTEDRDKPITQKISAEAWGRADC |
| domain (derived | | |
| from AEA41868) | | |
| Alb8 | 118 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVTVSS |
| Alb8-A | 119 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVTVSSA |
| Alb23 | 120 | EVQLLESGGGLVQPGGSLRLSCAASGFTFRSFGMSWVRQAPGKGPE |
| | | WVSSISGSGSDTLYADSVKGRFTISRDNSKNTLYLQMNSLRPEDTAVY |
| | | YCTIGGSLSRSSQGTLVTVSS |
| Alb23-A | 121 | EVQLLESGGGLVQPGGSLRLSCAASGFTFRSFGMSWVRQAPGKGPE |
| | | WVSSISGSGSDTLYADSVKGRFTISRDNSKNTLYLQMNSLRPEDTAVY |
| | | YCTIGGSLSRSSQGTLVTVSSA |
| Alb83 | 122 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTATY |
| | | YCTIGGSLSRSSQGTLVTVSS |
| Alb83-A | 123 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTATY |
| | | YCTIGGSLSRSSQGTLVTVSSA |
| Alb132 | 124 | EVQLVESGGGVVQPGGSLRLSCAASGFTFRSFGMSWVRQAPGKGP |
| | | EWVSSISGSGSDTLYADSVKGRFTISRDNSKNTLYLQMNSLRPEDTAT |
| | | YYCTIGGSLSRSSQGTLVTVSS |
| Alb132-A | 125 | EVQLVESGGGVVQPGGSLRLSCAASGFTFRSFGMSWVRQAPGKGP |
| | | EWVSSISGSGSDTLYADSVKGRFTISRDNSKNTLYLQMNSLRPEDTAT |
| | | YYCTIGGSLSRSSQGTLVTVSSA |
| Alb73 | 126 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVKVSS |
| Alb73-A | 127 | EVQLVESGGGLVQPGNSLRLSCAASGFTFSSFGMSWVRQAPGKGLE |
| | | WVSSISGSGSDTLYADSVKGRFTISRDNAKTTLYLQMNSLRPEDTAVY |
| | | YCTIGGSLSRSSQGTLVKVSSA |
| Alb82 | 128 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALY |
| | | YCTIGGSLSRSSQGTLVTVSS |
| Alb82-A | 129 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALY |
| | | YCTIGGSLSRSSQGTLVTVSSA |
| Alb199 | 130 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALY |
| | | YCTIGGSLSRSSQGTLVKVSS |
| Alb199-A | 131 | EVQLVESGGGVVQPGNSLRLSCAASGFTFSSFGMSWVRQAPGKGLE |
| | | WVSSISGSGSDTLYADSVKGRFTISRDNAKTTLYLQMNSLRPEDTALY |
| | | YCTIGGSLSRSSQGTLVKVSSA |
| Alb23002 | 132 | EVQLVESGGGVVQPGGSLRLSCAASGFTFRSFGMSWVRQAPGKGP |
| | | EWVSSISGSGSDTLYADSVKGRFTISRDNSKNTLYLQMNSLRPEDTAL |
| | | YYCTIGGSLSRSSQGTLVTVSS |
| Alb223 | 133 | EVQLVESGGGVVQPGGSLRLSCAASGFTFRSFGMSWVRQAPGKGP |
| | | EWVSSISGSGSDTLYADSVKGRFTISRDNSKNTLYLQMNSLRPEDTAL |
| | | YYCTIGGSLSRSSQGTLVTVSSA |
| Alb216 | 134 | EVQLVESGGGVVQPGGSLRLSCAASGFTFRSFGMSWVRQAPGKGP |
| | | EWVSSISGSGSDTLYADSVKGRFTISRDNSKNTLYLQMNSLRPEDTAL |
| | | YYCTIGGSLSRSSQGTLVKVSS |
| Alb216-A | 135 | EVQLVESGGGVVQPGGSLRLSCAASGFTFRSFGMSWVRQAPGKGP |
| | | EWVSSISGSGSDTLYADSVKGRFTISRDNSKNTLYLQMNSLRPEDTAL |
| | | YYCTIGGSLSRSSQGTLVKVSSA |
| 3A linker | 136 | AAA |
| 7GS linker | 137 | SGGSGGS |
| 8GS linker | 138 | GGGGSGGS |
| 10GS linker | 139 | GGGGSGGGGS |
| 15GS linker | 140 | GGGGSGGGGSGGGGS |
| 18GS linker | 141 | GGGGSGGGGSGGGGSGGS |
| 20GS linker | 142 | GGGGSGGGGSGGGGSGGGGS |
| 25GS linker | 143 | GGGGSGGGGSGGGGSGGGGSGGGGS |
| 30GS linker | 144 | GGGGSGGGGSGGGGSGGGGSGGGGSGGGGS |
| 35GS linker | 145 | GGGGSGGGGSGGGGSGGGGSGGGGSGGGGSGGGGS |
| 40GS linker | 146 | GGGGSGGGGSGGGGSGGGGSGGGGSGGGGSGGGGSGGGGS |
| G1 hinge | 147 | EPKSCDKTHTCPPCP |
| 9GS-G1 hinge | 148 | GGGGSGGGSEPKSCDKTHTCPPCP |
| Llama upper | 149 | EPKTPKPQPAAA |
| long hinge | | |
| region | | |
| G3 hinge | 150 | ELKTPLGDTTHTCPRCPEPKSCDTPPPCPRCPEPKSCDTPPPCPRCPE |
| | | PKSCDTPPPCPRCP |
| CDR2 of TCE | 151 | HISIGDQTDYADSAKG |
| ISVD (Kabat) | | |
| CDR2 of TCE | 152 | HISIGDQTDYANSAKG |
| ISVD (Kabat) | | |
| CDR2 of TCE | 153 | HISIGDQTDYAPSAKG |
| ISVD (Kabat) | | |
| CDR2 of TCE | 154 | HISIGDQTDYAKSAKG |
| ISVD (Kabat) | | |
| CDR2 of TCE | 155 | HISIGDQTDYARSAKG |
| ISVD (Kabat) | | |
| CDR2 of TCE | 156 | HISIGDQTDYAISAKG |
| ISVD (Kabat) | | |
| CDR2 of TCE | 157 | HISIGDQTDYATSAKG |
| ISVD (Kabat) | | |
| CDR2 of TCE | 158 | HISIGDQTDYAHSAKG |
| ISVD (Kabat) | | |
| CDR2 of TCE | 159 | HISIGDQTDYAVSAKG |
| ISVD (Kabat) | | |
| CDR2 of TCE | 160 | HISIGDQTDYAESAKG |
| ISVD (Kabat) | | |
| CDR2 of TCE | 161 | HISIGDQTDYAASAKG |
| ISVD (Kabat) | | |
| CDR2 of TCE | 162 | HISIGDQTDYAYSAKG |
| ISVD (Kabat) | | |
| CDR2 of TCE | 163 | HISIGDQTDYALSAKG |
| ISVD (Kabat) | | |
| CDR2 of TCE | 164 | HISIGDQTDYAQSAKG |
| ISVD (Kabat) | | |
| CDR2 of TCE | 165 | HISIGDQTDYAFSAKG |
| ISVD (Kabat) | | |
| CDR2 of TCE | 166 | HISIGDQTDYASSAKG |
| ISVD (Kabat) | | |
| CDR3 of TCE | 167 | LSRIWPYDY |
| ISVD (Kabat) | | |
| CDR3 of TCE | 168 | LSRIYPYDY |
| ISVD (Kabat) | | |
| CDR3 of TCE | 169 | LSRIAPYDY |
| ISVD (Kabat) | | |
| CDR3 of TCE | 170 | LSRIPPYDY |
| ISVD (Kabat) | | |
| CDR3 of TCE | 171 | LSRIDPYDY |
| ISVD (Kabat) | | |
| CDR3 of TCE | 172 | LSRIQPYDY |
| ISVD (Kabat) | | |
| CDR3 of TCE | 173 | LSRIEPYDY |
| ISVD (Kabat) | | |
| CDR3 of TCE | 174 | LSRIRPYDY |
| ISVD (Kabat) | | |
| CDR3 of TCE | 175 | LSRIFPYDY |
| ISVD (Kabat) | | |
| CDR3 of TCE | 176 | LSRISPYDY |
| ISVD (Kabat) | | |
| CDR3 of TCE | 177 | LSRIGPYDY |
| ISVD (Kabat) | | |
| CDR3 of TCE | 178 | LSRITPYDY |
| ISVD (Kabat) | | |
| CDR3 of TCE | 179 | LSRIHPYDY |
| ISVD (Kabat) | | |
| CDR3 of TCE | 180 | LSRIVPYDY |
| ISVD (Kabat) | | |
| CDR3 of TCE | 181 | LSRIKPYDY |
| ISVD (Kabat) | | |
| Peptide | 182 | VAHISIGDQTDYADSAK |
| fragment T7 | | |
| Peptide | 183 | NTVYLQMNSLRPEDTAAYYCR |
| fragment T11 | | |
| Peptide | 184 | IWPYDYWGQGTLVTVSSAAAHHHHHHH |
| fragment T13 | | |
| Peptide | 185 | LSCAASGFTFSSFAMTWVR |
| fragment T14 | | |
| Peptide | 186 | GLEWVATITNK |
| fragment T16 | | |
| Peptide | 187 | SVFSSSTMEWYR |
| fragment T27 | | |
| Peptide | 188 | NTVYLQMNSLRPEDTALYYCASGVAWGQGTLVTVSSGGGGSGGGS |
| fragment T36 | | EVQLVESGGGWQPGGS |
| Peptide | 189 | GPEWVSSISGSGSDTLYLADSVK |
| fragment T40 | | |

1. An immunoglobulin single variable domain (ISVD) which specifically binds to a constant domain of a human and/or non-human primate T cell receptor (TCR) present on a T cell, wherein said ISVD comprises 4 framework regions (FR1 to FR4 respectively) and 3 complementarity determining regions (CDR1 to CDR3 respectively), wherein:
a. the amino acid sequence of CDR1 (according to Kabat) is INFYG (SEQ ID NO: 79);
b. the amino acid sequence of CDR2 (according to Kabat) is HISIGDQTDYAX₁SAKG (SEQ ID NO: 80); and
c. the amino acid sequence of CDR3 (according to Kabat) is LSRIX₂PYDY (SEQ ID NO: 81);
wherein
the amino acid residue X₁ is selected from E, D, N, P, K, R, I, T, H, V, A, Y, L, Q, F, and S; and/or
the amino acid residue X₂ is selected from Y, A, P, D, Q, E, R, F, S, G, T, H, V, K, L and I.

2. An immunoglobulin single variable domain (ISVD) which specifically binds to a constant domain of a human and/or non-human primate T cell receptor (TCR) present on a T cell, wherein said ISVD comprises 4 framework regions (FR1 to FR4 respectively) and 3 complementarity determining regions (CDR1 to CDR3 respectively), wherein:
a. the amino acid sequence of CDR1 (according to AbM) is GYVHKINFYG (SEQ ID NO: 82);
b. the amino acid sequence of CDR2 (according to AbM) is HISIGDQTD (SEQ ID NO: 83); and
c. the amino acid sequence of CDR3 (according to AbM) is LSRIX₂PYDY (SEQ ID NO: 84);
and wherein
the amino acid residue at position 61 (according to Kabat) is selected from E, D, N, P, K, R, I, T, H, V, A, Y, L, Q, F, and S; and/or
the amino acid residue X₂ is selected from Y, A, P, D, Q, E, R, F, S, G, T, H, V, K, L and I.

3. The ISVD according to claim 1, wherein X₁ is E or D, optionally wherein X₁ is E.

4.-6. (canceled)

7. The ISVD according to claim 1, wherein X₂ is Y, A, Q, F, S, T or H, optionally wherein X₂ is Y, Q, S or T.

8. (canceled)

9. The ISVD according to claim 1, wherein X₂ is Y.

10. The ISVD according claim 1, wherein the amino acid residue in the ISVD at position 103 (Kabat numbering) is selected from the group consisting of W, R, A, E, Y, L, H, I, Q, V, K, S, G, P, F, and T, optionally wherein the amino acid residue in the ISVD at position 103 (Kabat numbering) is W.

11. (canceled)

12. The ISVD according to claim 1, wherein X₁ is E, X₂ is Y and the amino acid residue at position 103 (Kabat numbering) is W.

13. (canceled)

14. The ISVD according to claim 1, wherein the amino acid residue at position 1 (Kabat numbering) is selected from E and D.

15. The ISVD according to claim 1, wherein the ISVD is a heavy-chain ISVD, optionally wherein the heavy-chain ISVD is selected from a V_{HH}, a humanized V_{HH}, a camelized V_{H}, a domain antibody, a single domain antibody and a dAb.

16. (canceled)

17. The ISVD according to claim 1, that has a degree of sequence identity with the sequence of any of SEQ ID NOs: 2-57 of at least 85%, in which for the purposes of determining the degree of sequence identity, the amino acid residues that form the CDR sequences are disregarded, optionally wherein the sequence is SEQ ID NO: 37, SEQ ID NO: 42, SEQ ID NO: 46, SEQ ID NO: 50 or SEQ ID NO: 52.

18. The ISVD according to claim 1, that has a degree of sequence identity with the sequence of SEQ ID NOs: 32, 33 and/or 35-57 of at least 85%, in which for the purposes of determining the degree of sequence identity, the amino acid residues that form the CDR sequences are disregarded, optionally wherein the sequence comprises SEQ ID NO: 37, SEQ ID NO: 42, SEQ ID NO: 46, SEQ ID NO: 50 or SEQ ID NO: 52.

19.-20. (canceled)

21. An immunoglobulin single variable domain (ISVD), comprising or consisting of the following sequence:
(SEQ ID NO: 85)
X₀VQLVESGGGVVQPGGSLRLSCVASGYVHKINFYGWYRQAPGKE
REKVAHISIGDQTDYAX₁SAKGRFTISRDESKNTVYLQMNSLRPE
DTAAYYCRALSRIX₂PYDYX₃GQGTLVTVSS,
wherein
a. X₀ is selected from E and D.
b. X₁ is selected from the group consisting of E, D, N, P, K, R, I, T, H, V, A, Y, L, Q, F, and S;
c. X₂ is selected from the group consisting of Y, A, P, D, Q, E, R, F, S, G, T, H, V, K, L and I; and
d. X₃ is selected from the group consisting of W, R, A, E, Y, L, H, I, Q, V, K, S, G, P, F and T, optionally wherein:
X₀ is D;
X₁ is selected from the group consisting of D or E, optionally E;
X₂ is selected from the group consisting of Y, T, S and Q;
X₃ is W; or
any combination thereof.

22.-27. (canceled)

28. A polypeptide comprising a first ISVD capable of specifically binding to a constant domain of a human and/or non-human primate T cell receptor (TCR) present on a T cell and a second ISVD capable of specifically binding to a first antigen on a target cell, wherein said first antigen is different from said TCR, and wherein said target cell is different from said T cell, wherein said first and second ISVD essentially consist of 4 framework regions (FR1 to FR4 respectively) and 3 complementarity determining regions (CDR1 to CDR3 respectively), and wherein the first ISVD is an ISVD according to claim 1.

29. The polypeptide according to claim 28, wherein the amino acid sequence of the first ISVD has at least 80% sequence identity with at least one of the amino acid sequence of any of SEQ ID NOs: 2-57, in which for the purposes of determining the degree of sequence identity, the amino acid residues that form the CDR sequences are disregarded.

30.-32. (canceled)

33. The polypeptide according to claim 28, further comprising:
a third ISVD, which specifically binds to a second antigen on a target cell;
one or more other groups, residues, moieties or binding units, optionally linked via one or more peptidic linkers, in which said one or more other groups, residues, moieties or binding units provide the polypeptide with increased half-life, compared to the corresponding polypeptide without said one or more other groups, residues, moieties or binding units, optionally wherein said one or more other groups, residues, moieties or binding units is an ISVD that can bind to human serum albumin; or
a combination thereof.

34.-35. (canceled)

36. A method of producing an ISVD according to claim 1 or a polypeptide comprising said ISVD, wherein the method comprises:
a. expressing, in a suitable host cell or host organism or in another suitable expression system, a nucleic acid sequence encoding the ISVD or polypeptide; optionally followed by:
b. isolating and/or purifying the ISVD or polypeptide.

37. A nucleic acid comprising a sequence encoding the ISVD according to claim 1.

38. (canceled)

39. A non-human host or host cell transformed or transfected with the nucleic acid according to claim 37.

40. A composition comprising the ISVD according to claim 1 or a polypeptide comprising the same, optionally wherein the composition is a pharmaceutical composition.

41.-42. (canceled)

43. A method for the treatment of a disease, comprising administering to a subject in need thereof a pharmaceutically active amount of an ISVD according to claim 1 or a polypeptide comprising the same, wherein the disease is selected from the group consisting of a proliferative disease, an inflammatory disease, an infectious disease and an autoimmune disease, optionally wherein the disease is cancer.

44. (canceled)


SEQUENCE LISTING (237 sequences)

SEQ ID NO: 1 - protein, 117 aa, synthetic construct
  REGION 1..117: note = T017000700
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 2 - protein, 117 aa, synthetic construct
  REGION 1..117: note = T017000937
EVQLVESGGGVVQPGGSLRLSCVASGYVHKINFYGWYRQAPGKEREKVAHISIGDQTDYANSAKGRFTISRDESKNTVYLQMNSLRPEDTAAYYCRALSRIWPYDYWGQGTLVTVSS

SEQ ID NO: 3 - protein, 117 aa, synthetic construct
  REGION 1..117: note = T017000938
EVQLVESGGGVVQPGGSLRLSCVASGYVHKINFYGWYRQAPGKEREKVAHISIGDQTDYAPSAKGRFTISRDESKNTVYLQMNSLRPEDTAAYYCRALSRIWPYDYWGQGTLVTVSS

SEQ ID NO: 4 - protein, 117 aa, synthetic construct
  REGION 1..117: note = T017000939
EVQLVESGGGVVQPGGSLRLSCVASGYVHKINFYGWYRQAPGKEREKVAHISIGDQTDYAKSAKGRFTISRDESKNTVYLQMNSLRPEDTAAYYCRALSRIWPYDYWGQGTLVTVSS

SEQ ID NO: 5 - protein, 117 aa, synthetic construct
  REGION 1..117: note = T017000940
EVQLVESGGGVVQPGGSLRLSCVASGYVHKINFYGWYRQAPGKEREKVAHISIGDQTDYARSAKGRFTISRDESKNTVYLQMNSLRPEDTAAYYCRALSRIWPYDYWGQGTLVTVSS

SEQ ID NO: 6 - protein, 117 aa, synthetic construct
  REGION 1..117: note = T017000941
EVQLVESGGGVVQPGGSLRLSCVASGYVHKINFYGWYRQAPGKEREKVAHISIGDQTDYAISAKGRFTISRDESKNTVYLQMNSLRPEDTAAYYCRALSRIWPYDYWGQGTLVTVSS

SEQ ID NO: 7 - protein, 117 aa, synthetic construct
  REGION 1..117: note = T017000942
EVQLVESGGGVVQPGGSLRLSCVASGYVHKINFYGWYRQAPGKEREKVAHISIGDQTDYATSAKGRFTISRDESKNTVYLQMNSLRPEDTAAYYCRALSRIWPYDYWGQGTLVTVSS

SEQ ID NO: 8 - protein, 117 aa, synthetic construct
  REGION 1..117: note = T017000943
EVQLVESGGGVVQPGGSLRLSCVASGYVHKINFYGWYRQAPGKEREKVAHISIGDQTDYAHSAKGRFTISRDESKNTVYLQMNSLRPEDTAAYYCRALSRIWPYDYWGQGTLVTVSS

SEQ ID NO: 9 - protein, 117 aa, synthetic construct
  REGION 1..117: note = T017000944
EVQLVESGGGVVQPGGSLRLSCVASGYVHKINFYGWYRQAPGKEREKVAHISIGDQTDYAVSAKGRFTISRDESKNTVYLQMNSLRPEDTAAYYCRALSRIWPYDYWGQGTLVTVSS

SEQ ID NO: 10 - protein, 117 aa, synthetic construct
  REGION 1..117: note = T017000945
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIWPYDYWGQGTLVTVSS

SEQ ID NO: 11 - protein, 117 aa, synthetic construct
  REGION 1..117: note = T017000946
EVQLVESGGGVVQPGGSLRLSCVASGYVHKINFYGWYRQAPGKEREKVAHISIGDQTDYAASAKGRFTISRDESKNTVYLQMNSLRPEDTAAYYCRALSRIWPYDYWGQGTLVTVSS

SEQ ID NO: 12 - protein, 117 aa, synthetic construct
  REGION 1..117: note = T017000947
EVQLVESGGGVVQPGGSLRLSCVASGYVHKINFYGWYRQAPGKEREKVAHISIGDQTDYAYSAKGRFTISRDESKNTVYLQMNSLRPEDTAAYYCRALSRIWPYDYWGQGTLVTVSS

SEQ ID NO: 13 - protein, 117 aa, synthetic construct
  REGION 1..117: note = T017000948
EVQLVESGGGVVQPGGSLRLSCVASGYVHKINFYGWYRQAPGKEREKVAHISIGDQTDYALSAKGRFTISRDESKNTVYLQMNSLRPEDTAAYYCRALSRIWPYDYWGQGTLVTVSS

SEQ ID NO: 14 - protein, 117 aa, synthetic construct
  REGION 1..117: note = T017000949
EVQLVESGGGVVQPGGSLRLSCVASGYVHKINFYGWYRQAPGKEREKVAHISIGDQTDYAQSAKGRFTISRDESKNTVYLQMNSLRPEDTAAYYCRALSRIWPYDYWGQGTLVTVSS

SEQ ID NO: 15 - protein, 117 aa, synthetic construct
  REGION 1..117: note = T017000950
EVQLVESGGGVVQPGGSLRLSCVASGYVHKINFYGWYRQAPGKEREKVAHISIGDQTDYAFSAKGRFTISRDESKNTVYLQMNSLRPEDTAAYYCRALSRIWPYDYWGQGTLVTVSS

SEQ ID NO: 16 - protein, 117 aa, synthetic construct
  REGION 1..117: note = T017000951
EVQLVESGGGVVQPGGSLRLSCVASGYVHKINFYGWYRQAPGKEREKVAHISIGDQTDYASSAKGRFTISRDESKNTVYLQMNSLRPEDTAAYYCRALSRIWPYDYWGQGTLVTVSS

SEQ ID NO: 17 - protein, 117 aa, synthetic construct
  REGION 1..117: note = T017000952
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYRGQGTLVTVSS

SEQ ID NO: 18 - protein, 117 aa, synthetic construct
  REGION 1..117: note = T017000953
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYAGQGTLVTVSS

SEQ ID NO: 19 - protein, 117 aa, synthetic construct
  REGION 1..117: note = T017000954
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYEGQGTLVTVSS

SEQ ID NO: 20 - protein, 117 aa, synthetic construct
  REGION 1..117: note = T017000955
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYYGQGTLVTVSS

SEQ ID NO: 21 - protein, 117 aa, synthetic construct
  REGION 1..117: note = T017000956
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYLGQGTLVTVSS

SEQ ID NO: 22 - protein, 117 aa, synthetic construct
  REGION 1..117: note = T017000957
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYHGQGTLVTVSS

SEQ ID NO: 23 - protein, 117 aa, synthetic construct
  REGION 1..117: note = T017000958
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYIGQGTLVTVSS

SEQ ID NO: 24 - protein, 117 aa, synthetic construct
  REGION 1..117: note = T017000959
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYQGQGTLVTVSS

SEQ ID NO: 25 - protein, 117 aa, synthetic construct
  REGION 1..117: note = T017000960
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYVGQGTLVTVSS

SEQ ID NO: 26 - protein, 117 aa, synthetic construct
  REGION 1..117: note = T017000961
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYKGQGTLVTVSS

SEQ ID NO: 27 - protein, 117 aa, synthetic construct
  REGION 1..117: note = T017000962
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYSGQGTLVTVSS

SEQ ID NO: 28 - protein, 117 aa, synthetic construct
  REGION 1..117: note = T017000963
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYGGQGTLVTVSS

SEQ ID NO: 29 - protein, 117 aa, synthetic construct
  REGION 1..117: note = T017000964
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYPGQGTLVTVSS

SEQ ID NO: 30 - protein, 117 aa, synthetic construct
  REGION 1..117: note = T017000965
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYFGQGTLVTVSS

SEQ ID NO: 31 - protein, 117 aa, synthetic construct
  REGION 1..117: note = T017000966
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYTGQGTLVTVSS

SEQ ID NO: 32 - protein, 117 aa, synthetic construct
  REGION 1..117: note = T017000967
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIYPYDYWGQGTLVTVSS

SEQ ID NO: 33 - protein, 117 aa, synthetic construct
  REGION 1..117: note = T017000974
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSS

SEQ ID NO: 34 - protein, 117 aa, synthetic construct
  REGION 1..117: note = T017000975
EVQLVESGGGVVQPGGSLRLSCVASGYVHKINFYGWYRQAPGKEREKVAHISIGDQTDYAPSAKGRFTISRDESKNTVYLQMNSLRPEDTAAYYCRALSRIYPYDYWGQGTLVTVSS

SEQ ID NO: 35 - protein, 117 aa, synthetic construct
  REGION 1..117: note = T017000976
EVQLVESGGGVVQPGGSLRLSCVASGYVHKINFYGWYRQAPGKEREKVAHISIGDQTDYARSAKGRFTISRDESKNTVYLQMNSLRPEDTAAYYCRALSRIYPYDYWGQGTLVTVSS

SEQ ID NO: 36 - protein, 117 aa, synthetic construct
  REGION 1..117: note = T017000977
EVQLVESGGGVVQPGGSLRLSCVASGYVHKINFYGWYRQAPGKEREKVAHISIGDQTDYAVSAKGRFTISRDESKNTVYLQMNSLRPEDTAAYYCRALSRIYPYDYWGQGTLVTVSS

SEQ ID NO: 37 - protein, 117 aa, synthetic construct
  REGION 1..117: note = T017000978
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYWGQGTLVTVSS

SEQ ID NO: 38 - protein, 117 aa, synthetic construct
  REGION 1..117: note = T017000979
EVQLVESGGGVVQPGGSLRLSCVASGYVHKINFYGWYRQAPGKEREKVAHISIGDQTDYAASAKGRFTISRDESKNTVYLQMNSLRPEDTAAYYCRALSRIYPYDYWGQGTLVTVSS

SEQ ID NO: 39 - protein, 117 aa, synthetic construct
  REGION 1..117: note = T017000980
EVQLVESGGGVVQPGGSLRLSCVASGYVHKINFYGWYRQAPGKEREKVAHISIGDQTDYAQSAKGRFTISRDESKNTVYLQMNSLRPEDTAAYYCRALSRIYPYDYWGQGTLVTVSS

SEQ ID NO: 40 - protein, 117 aa, synthetic construct
  REGION 1..117: note = T017000981
EVQLVESGGGVVQPGGSLRLSCVASGYVHKINFYGWYRQAPGKEREKVAHISIGDQTDYASSAKGRFTISRDESKNTVYLQMNSLRPEDTAAYYCRALSRIYPYDYWGQGTLVTVSS

SEQ ID NO: 41 - protein, 117 aa, synthetic construct
  REGION 1..117: note = T017000982
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYYGQGTLVTVSS

SEQ ID NO: 42 - protein, 117 aa, synthetic construct
  REGION 1..117: note = T017000991
DVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYWGQGTLVTVSS

SEQ ID NO: 43 - protein, 117 aa, synthetic construct
  REGION 1..117: note = T017000992
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIAPYDYWGQGTLVTVSS

SEQ ID NO: 44 - protein, 117 aa, synthetic construct
  REGION 1..117: note = T017000993
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIPPYDYWGQGTLVTVSS

SEQ ID NO: 45 - protein, 117 aa, synthetic construct
  REGION 1..117: note = T017000994
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIDPYDYWGQGTLVTVSS

SEQ ID NO: 46 - protein, 117 aa, synthetic construct
  REGION 1..117: note = T017000995
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIQPYDYWGQGTLVTVSS

SEQ ID NO: 47 - protein, 117 aa, synthetic construct
  REGION 1..117: note = T017000996
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIEPYDYWGQGTLVTVSS

SEQ ID NO: 48 - protein, 117 aa, synthetic construct
  REGION 1..117: note = T017000997
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIRPYDYWGQGTLVTVSS

SEQ ID NO: 49 - protein, 117 aa, synthetic construct
  REGION 1..117: note = T017000998
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIFPYDYWGQGTLVTVSS

SEQ ID NO: 50 - protein, 117 aa, synthetic construct
  REGION 1..117: note = T017000999
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRISPYDYWGQGTLVTVSS

SEQ ID NO: 51 - protein, 117 aa, synthetic construct
  REGION 1..117: note = T017001000
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIGPYDYWGQGTLVTVSS

SEQ ID NO: 52 - protein, 117 aa, synthetic construct
  REGION 1..117: note = T017001001
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRITPYDYWGQGTLVTVSS

SEQ ID NO: 53 - protein, 117 aa, synthetic construct
  REGION 1..117: note = T017001002
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIHPYDYWGQGTLVTVSS

SEQ ID NO: 54 - protein, 117 aa, synthetic construct
  REGION 1..117: note = T017001003
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIVPYDYWGQGTLVTVSS

SEQ ID NO: 55 - protein, 116 aa, synthetic construct
  REGION 1..116: note = T017001004
EVQLVESGGGVVQPGGSLRLSCVASGYVHKINFYGWYRQAPGKEREKVAHISIGDQTDYASAKGRFTISRDESKNTVYLQMNSLRPEDTAAYYCRALSRIKPYDYWGQGTLVTVSS

SEQ ID NO: 56 - protein, 117 aa, synthetic construct
  REGION 1..117: note = T017001005
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRILPYDYWGQGTLVTVSS

SEQ ID NO: 57 - protein, 117 aa, synthetic construct
  REGION 1..117: note = T017001006
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIIPYDYWGQGTLVTVSS

SEQ ID NO: 58 - protein, 377 aa, synthetic construct
  REGION 1..377: note = T017001017
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 59 - protein, 377 aa, synthetic construct
  REGION 1..377: note = T017001018
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 60 - protein, 377 aa, synthetic construct
  REGION 1..377: note = T017001019
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRITPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 61 - protein, 377 aa, synthetic construct
  REGION 1..377: note = T017001020
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRISPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 62 - protein, 377 aa, synthetic construct
  REGION 1..377: note = T017001021
EVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIQPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 63 - protein, 377 aa, synthetic construct
  REGION 1..377: note = T017001022
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 64 - protein, 377 aa, synthetic construct
  REGION 1..377: note = T017001023
DVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 65 - protein, 377 aa, synthetic construct
  REGION 1..377: note = T017001024
DVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRITPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 66 - protein, 377 aa, synthetic construct
  REGION 1..377: note = T017001025
DVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRISPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 67 - protein, 377 aa, synthetic construct
  REGION 1..377: note = T017001026
DVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIQPYDYWGQGTLVTVSSGGGGSGGGSEVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 68 - protein, 377 aa, synthetic construct
  REGION 1..377: note = T017001027
EVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 69 - protein, 377 aa, synthetic construct
  REGION 1..377: note = T017001028
EVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 70 - protein, 377 aa, synthetic construct
  REGION 1..377: note = T017001029
EVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRITPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 71 - protein, 377 aa, synthetic construct
  REGION 1..377: note = T017001030
EVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRISPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 72 - protein, 377 aa, synthetic construct
  REGION 1..377: note = T017001031
EVQLVESGGGVVQPGGSLRLSCAASGTTLDYYVIGWFRQAPGKEREGVSCISSSDGYTYYADSVKGRFTISRDNAKNTVYLQMNSLRPEDTALYYCATDPYYSSCHPFFADYEYDYWGQGTLVTVSSGGGGSGGGSEVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIQPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 73 - protein, 498 aa, synthetic construct
  REGION 1..498: note = A022600427
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGFTFSSFAMTWVRRPPGKGLEWVATITNAGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 74 - protein, 498 aa, synthetic construct
  REGION 1..498: note = A022600462
DVQLVESGGGVVQPGGSLRLSCVASGYVHKINFYGWYRQAPGKEREKVAHISIGDQTDYAESAKGRFTISRDESKNTVYLQMNSLRPEDTAAYYCRALSRIYPYDYWGQGTLVTVSSGGGGSEVQLVESGGGVVQPGGSLRLSCAASGFTFSSFAMTWVRRPPGKGLEWVATITNKGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 75 - protein, 242 aa, synthetic construct
  REGION 1..242: note = T017000698
DVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 76 - protein, 497 aa, synthetic construct
  REGION 1..497: note = A022600424
VQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGGGSEVQLVESGGGVVQPGGSLRLSCAASGFTFSSFAMTWVRRPPGKGLEWVATITNKGVTSYADSVKGRFTISRDNAKNTLYLQMNSLRPEDTALYICANARRTGPRAPTDIGSYRGQGTLVTVSSGGGGSGGGSEVQLVESGGGVVQPGGSLRLSCAASGSIFRSVFSSSTMEWYRQAPGKKRELVARIAPGEGTYYGALYADSVKGRFTISRDNAKNTVYLQMNSLRPEDTALYYCASGVAWGQGTLVTVSSGGGGSGGGSEVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 77 - protein, 34 aa, synthetic construct
  REGION 1..34: note = FLAG3-HIS6 tag
AAADYKDHDGDYKDHDIDYKDDDDKGAAHHHHHH

SEQ ID NO: 78 - protein, 6 aa, synthetic construct
  REGION 1..6: note = HIS6 tag
HHHHHH

SEQ ID NO: 79 - protein, 5 aa, synthetic construct
  REGION 1..5: note = CDR1 of TCE ISVD (Kabat)
INFYG

SEQ ID NO: 80 - protein, 16 aa, synthetic construct
  REGION 1..16: note = CDR2 of TCE ISVD (Kabat)
  SITE 12: note = X is selected from E, D, N, P, K, R, I, T, H, V, A, Y, L, Q, F, and S
HISIGDQTDYAXSAKG

SEQ ID NO: 81 - protein, 9 aa, synthetic construct
  REGION 1..9: note = CD3 of TCE ISVD (Kabat)
  SITE 5: note = X is selected from Y, A, P, D, Q, E, R, F, S G, T, H, V, K, L, and I
LSRIXPYDY

SEQ ID NO: 82 - protein, 10 aa, synthetic construct
  REGION 1..10: note = CDR1 of TCE ISVD (Abm)
GYVHKINFYG

SEQ ID NO: 83 - protein, 9 aa, synthetic construct
  REGION 1..9: note = CDR2 of TCE ISVD (Abm)
HISIGDQTD

SEQ ID NO: 84 - protein, 9 aa, synthetic construct
  REGION 1..9: note = CDR3 of TCE ISVD (Abm)
  SITE 5: note = X is selected from Y, A, P, D, Q, E, R, F, S G, T, H, V, K, L, and I.
LSRIXPYDY

SEQ ID NO: 85 - protein, 117 aa, synthetic construct
  SITE 1: note = X is selected from E and D
  REGION 1..117: note = TCE ISVD
  SITE 61: note = X is selected from the group consisting of E, D, N, P, K, R, I, T, H, V, A, Y, L, Q, F, and S
  SITE 102: note = X is selected from the group consisting of Y, A, P, D, Q, E, R, F, S, G, T, H, V, K, L and I
  SITE 107: note = X is selected from the group consisting of W, R, A, E, Y, L, H, I, Q, V, K, S, G, P, F and T.
XVQLVESGGGVVQPGGSLRLSCVASGYVHKINFYGWYRQAPGKEREKVAHISIGDQTDYAXSAKGRFTISRDESKNTVYLQMNSLRPEDTAAYYCRALSRIXPYDYXGQGTLVTVSS

SEQ ID NO: 86 - protein, 5 aa, synthetic construct
  REGION 1..5: note = 5GS linker
GGGGS

SEQ ID NO: 87 - protein, 9 aa, synthetic construct
  REGION 1..9: note = 9GS linker
GGGGSGGGS

SEQ ID NO: 88 - protein, 5 aa, synthetic construct
  REGION 1..5: note = C-terminus ISVD
VTVSS

SEQ ID NO: 89 - protein, 5 aa, synthetic construct
  REGION 1..5: note = C-terminus ISVD
VKVSS

SEQ ID NO: 90 - protein, 5 aa, synthetic construct
  REGION 1..5: note = C-terminus ISVD
VQVSS

SEQ ID NO: 91 - protein, 5 aa, synthetic construct
  REGION 1..5: note = C-terminus ISVD
VTVKS

SEQ ID NO: 92 - protein, 5 aa, synthetic construct
  REGION 1..5: note = C-terminus ISVD
VTVQS

SEQ ID NO: 93 - protein, 5 aa, synthetic construct
  REGION 1..5: note = C-terminus ISVD
VKVKS

SEQ ID NO: 94 - protein, 5 aa, synthetic construct
  REGION 1..5: note = C-terminus ISVD
VKVQS

SEQ ID NO: 95 - protein, 5 aa, synthetic construct
  REGION 1..5: note = C-terminus ISVD
VQVKS

SEQ ID NO: 96 - protein, 5 aa, synthetic construct
  REGION 1..5: note = C-terminus ISVD
VQVQS

SEQ ID NO: 97 - protein, 6 aa, synthetic construct
  REGION 1..6: note = C-terminus ISVD
VTVSSA

SEQ ID NO: 98 - protein, 6 aa, synthetic construct
  REGION 1..6: note = C-terminus ISVD
VKVSSA

SEQ ID NO: 99 - protein, 6 aa, synthetic construct
  REGION 1..6: note = C-terminus ISVD
VQVSSA

SEQ ID NO: 100 - protein, 6 aa, synthetic construct
  REGION 1..6: note = C-terminus ISVD
VTVKSA

SEQ ID NO: 101 - protein, 6 aa, synthetic construct
  REGION 1..6: note = C-terminus ISVD
VTVQSA

SEQ ID NO: 102 - protein, 6 aa, synthetic construct
  REGION 1..6: note = C-terminus ISVD
VKVKSA

SEQ ID NO: 103 - protein, 6 aa, synthetic construct
  REGION 1..6: note = C-terminus ISVD
VKVQSA

SEQ ID NO: 104 - protein, 6 aa, synthetic construct
  REGION 1..6: note = C-terminus ISVD
VQVKSA

SEQ ID NO: 105 - protein, 6 aa, synthetic construct
  REGION 1..6: note = C-terminus ISVD
VQVQSA

SEQ ID NO: 106 - protein, 142 aa, synthetic construct
  REGION 1..142: note = Human TCR alpha constant domain (derived from P01848)
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 107 - protein, 177 aa, synthetic construct
  REGION 1..177: note = Human TCR beta constant domain (derived from P01850)
EDLNKVFPPEVAVFEPSEAEISHTQKATLVCLATGFFPDHVELSWWVNGKEVHSGVSTDPQPLKEQPALNDSRYALSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 108 - protein, 270 aa, synthetic construct
  REGION 1..270: note = Rhesus TCR alpha (AEA41863)
MLLLLVLVLEVIFTLGGTRAQSVTQLDSQVSVSEGVPVLLRCNYSSSFSPYLFWYVQYPNQGLQLLLKYTSGTTLVKGINGFEAEFKKSETSFHLTKASAHVSDAAEYFCALARGALVFGKGTRLSVIPNIQNPDPAVYQLRGSKSNDTSVCLFTDFDSVMNVSQSKDSDVHITDKTVLDMRSMDFKSNGAVAWSNKSDFACTSAFKDSVIPADTFFPGTESVCDANLVEKSFETDMNLNFQNLSVIGFRILLLKVAGFNLLMTLRLWSS

SEQ ID NO: 109 - protein, 310 aa, synthetic construct
  REGION 1..310: note = Rhesus TCR beta (AEA41864)
MGFWTLCCVSFCILVAKHTDAGVIQLPRHEVTEMGKEVTLRCEPISGHSSLFWYRQTMMRGLEFLIYFNNKSPIDDSGMPKDRFSATMPDASFSTLKIQPSEPRDSAVYFCASTPGQGREKLFFGSGTQLSVLEDLKKVFPPKVAVFEPSEAEISHTQKATLVCLATGFYPDHVELSWWVNGKEVHSGVSTDPQPLKEQPALEDSRYCLSSRLRVSATFWHNPRNHFRCQVQFYGLSEDDEWTEDRDKPITQKISAEVWGRADCGFTSVSYQQGVLSATILYEILLGKATLYAVLVSALMLMAMVKRKDF

SEQ ID NO: 110 - protein, 281 aa, synthetic construct
  REGION 1..281: note = Human TCR-zipper: alpha chain
MNMRPVTSSVLVLLLMLRRSNGQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKSVLDMRSMDFKSNSAVAWSNKSDFACANAFNNSIIPEDTFFPSPESSCSSADLVPRGSTTAPSAQLKKKLQALKKKNAQLKWKLQALKKKLAQEQKLISEEDL

SEQ ID NO: 111 - protein, 329 aa, synthetic construct
  REGION 1..329: note = Human TCR-zipper: beta chain
MSNTVLADSAWGITLLSWVTVFLLGTSSADGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYSLSSRLRVSATFWQNPRNHFRCQVQFYGLSENDEWTQDRAKPVTQIVSAEAWGRADCSSADLVPRGSTTAPSAQLEKELQALEKENAQLEWELQALEKELAQTGHHHHHHHHHH

SEQ ID NO: 112 - protein, 277 aa, synthetic construct
  REGION 1..277: note = Cyno TCR-zipper: alpha chain (derived from AEA41865)
MLLITSVLVLWMQLSQVNGQQIMQIPQYQHVQEGEDFTTYCNSSTTLSNIQWYKQRPGGHPVFLIMLVKSGEVKKQKRLIFQFGEAKKNSSLHITATQTTDVGTYFCATTGVNNLFFGTGTRLTVLPYIQNPDPAVYQLRGSKSNDTSVCLFTDFDSVMNVSQSKDSDVHITDKTVLDMRSMDFKSNGAVAWSNKSDFACTSAFKDSVIPADTFFPSPESSCSSADLVPRGSTTAPSAQLKKKLQALKKKNAQLKWKLQALKKKLAQEQKLISEEDL

SEQ ID NO: 113 - protein, 321 aa, synthetic construct
  REGION 1..321: note = Cyno TCR-zipper: beta chain (derived from AEA41866 and AEA41868)
MSIGLLCCAALSLLWAGPVNAGVTQTPKFQVLKTGQSMTLQCAQDMNHDYMYWYRQDPGMGLRLIHYSVGEGSTEKGEVPDGYNVTRSNTEDFPLRLESAAPSQTSVYFCASSYWTGRSYEQYFGPGTRLTVIEDLKKVFPPKVAVFEPSEAEISHTQKATLVCLATGFYPDHVELSWWVNGKEVHSGVSTDPQPLKEQPALEDSRYSLSSRLRVSATFWHNPRNHFRCQVQFYGLSEDDEWTEDRDKPITQKISAEAWGRADCSSADLVPRGSTTAPSAQLEKELQALEKENAQLEWELQALEKELAQTGHHHHHHHHHH

SEQ ID NO: 114 - protein, 96 aa, synthetic construct
  REGION 1..96: note = Human TCR alpha constant domain (derived from P01848)
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSC

SEQ ID NO: 115 - protein, 131 aa, synthetic construct
  REGION 1..131: note = Human TCR beta constant domain (derived from P01850)
EDLNKVFPPEVAVFEPSEAEISHTQKATLVCLATGFFPDHVELSWWVNGKEVHSGVSTDPQPLKEQPALNDSRYALSSRLRVSATFWQNPRNHFRCQVQFYGLSENDEWTQDRAKPVTQIVSAEAWGRADC

SEQ ID NO: 116 - protein, 96 aa, synthetic construct
  REGION 1..96: note = cynomolgus TCR-alpha constant domain (derived from AEA41865)
PYIQNPDPAVYQLRGSKSNDTSVCLFTDFDSVMNVSQSKDSDVHITDKTVLDMRSMDFKSNGAVAWSNKSDFACTSAFKDSVIPADTFFPSPESSC

SEQ ID NO: 117 - protein, 131 aa, synthetic construct
  REGION 1..131: note = cynomolgus TCR-beta constant domain (derived from AEA41868)
EDLKKVFPPKVAVFEPSEAEISHTQKATLVCLATGFYPDHVELSWWVNGKEVHSGVSTDPQPLKEQPALEDSRYSLSSRLRVSATFWHNPRNHFRCQVQFYGLSEDDEWTEDRDKPITQKISAEAWGRADC

SEQ ID NO: 118 - protein, 115 aa, synthetic construct [sequence omitted]

SEQ ID NO: 119 - protein, 116 aa, synthetic construct
  REGION 1..116: note = Alb8-A
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSA

SEQ ID NO: 120 - protein, 115 aa, synthetic construct [sequence omitted]

SEQ ID NO: 121 - protein, 116 aa, synthetic construct
  REGION 1..116: note = Alb23-A
EVQLLESGGGLVQPGGSLRLSCAASGFTFRSFGMSWVRQAPGKGPEWVSSISGSGSDTLYADSVKGRFTISRDNSKNTLYLQMNSLRPEDTAVYYCTIGGSLSRSSQGTLVTVSSA

SEQ ID NO: 122 - protein, 115 aa, synthetic construct
  REGION 1..115: note = Alb83
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSS

SEQ ID NO: 123 - protein, 116 aa, synthetic construct
  REGION 1..116: note = Alb83-A
EVQLVESGGGVVQPGNSLRLSCAASGFTFSSFGMSWVRQAPGKGLEWVSSISGSGSDTLYADSVKGRFTISRDNAKTTLYLQMNSLRPEDTATYYCTIGGSLSRSSQGTLVTVSSA

SEQ ID NO: 124 - protein, 115 aa, synthetic construct
  REGION 1..115: note = Alb132
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSS

SEQ ID NO: 125 - protein, 116 aa, synthetic construct
  REGION 1..116: note = Alb132-A
EVQLVESGGGVVQPGGSLRLSCAASGFTFRSFGMSWVRQAPGKGPEWVSSISGSGSDTLYADSVKGRFTISRDNSKNTLYLQMNSLRPEDTATYYCTIGGSLSRSSQGTLVTVSSA

SEQ ID NO: 126 - protein, 115 aa, synthetic construct
  REGION 1..115: note = Alb73
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSS

SEQ ID NO: 127 - protein, 116 aa, synthetic construct
  REGION 1..116: note = Alb73-A
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVKVSSA

SEQ ID NO: 128 - protein, 115 aa, synthetic construct
  REGION 1..115: note = Alb82
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSS

SEQ ID NO: 129 - protein, 116 aa, synthetic construct
  REGION 1..116: note = Alb82-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVTVSSA

SEQ ID NO: 130 - protein, 115 aa, synthetic construct
  REGION 1..115: note = Alb199
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVKVSS

SEQ ID NO: 131 - protein, 116 aa, synthetic construct
  REGION 1..116: note = Alb199-A
EVQLVESGGGVVQPGNSLRLSCAASGFTFSSFGMSWVRQAPGKGLEWVSSISGSGSDTLYADSVKGRFTISRDNAKTTLYLQMNSLRPEDTALYYCTIGGSLSRSSQGTLVKVSSA

SEQ ID NO: 132 - protein, 115 aa, synthetic construct
  REGION 1..115: note = Alb23002
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSS

SEQ ID NO: 133 - protein, 116 aa, synthetic construct
  REGION 1..116: note = Alb223
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVTVSSA

SEQ ID NO: 134 - protein, 115 aa, synthetic construct
  REGION 1..115: note = Alb216
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVKVSS

SEQ ID NO: 135 - protein, 116 aa, synthetic construct
  REGION 1..116: note = Alb216-A
EVQLVESGGGVVQPGGSLRLSCAASGFTFRSFGMSWVRQAPGKGPEWVSSISGSGSDTLYADSVKGRFTISRDNSKNTLYLQMNSLRPEDTALYYCTIGGSLSRSSQGTLVKVSSA

SEQ ID NO: 136 - skipped

SEQ ID NO: 137 - protein, 7 aa, synthetic construct
  REGION 1..7: note = 7GS linker
SGGSGGS

SEQ ID NO: 138 - protein, 8 aa, synthetic construct
  REGION 1..8: note = 8GS linker
GGGGSGGS

SEQ ID NO: 139 - protein, 10 aa, synthetic construct
  REGION 1..10: note = 10GS linker
GGGGSGGGGS

SEQ ID NO: 140 - protein, 15 aa, synthetic construct
  REGION 1..15: note = 15GS linker
GGGGSGGGGSGGGGS

SEQ ID NO: 141 - protein, 18 aa, synthetic construct
  REGION 1..18: note = 18GS linker
GGGGSGGGGSGGGGSGGS

SEQ ID NO: 142 - protein, 20 aa, synthetic construct
  REGION 1..20: note = 20GS linker
GGGGSGGGGSGGGGSGGGGS

SEQ ID NO: 143 - protein, 25 aa, synthetic construct
  REGION 1..25: note = 25GS linker
GGGGSGGGGSGGGGSGGGGSGGGGS

SEQ ID NO: 144 - protein, 30 aa, synthetic construct
  REGION 1..30: note = 30GS linker
GGGGSGGGGSGGGGSGGGGSGGGGSGGGGS

SEQ ID NO: 145 - protein, 35 aa, synthetic construct
  REGION 1..35: note = 35GS linker
GGGGSGGGGSGGGGSGGGGSGGGGSGGGGSGGGGS

SEQ ID NO: 146 - protein, 40 aa, synthetic construct
  REGION 1..40: note = 40GS linker
GGGGSGGGGSGGGGSGGGGSGGGGSGGGGSGGGGSGGGGS

SEQ ID NO: 147 - protein, 15 aa, synthetic construct
  REGION 1..15: note = G1 hinge
EPKSCDKTHTCPPCP

SEQ ID NO: 148 - protein, 24 aa, synthetic construct
  REGION 1..24: note = 9GS-G1 hinge
GGGGSGGGSEPKSCDKTHTCPPCP

SEQ ID NO: 149 - protein, 12 aa, synthetic construct
  REGION 1..12: note = Llama upper long hinge region
EPKTPKPQPAAA

SEQ ID NO: 150 - protein, 62 aa, synthetic construct
  REGION 1..62: note = G3 hinge
ELKTPLGDTTHTCPRCPEPKSCDTPPPCPRCPEPKSCDTPPPCPRCPEPKSCDTPPPCPRCP

SEQ ID NO: 151 - protein, 16 aa, synthetic construct
  REGION 1..16: note = CDR2 of TCE ISVD (Kabat)
HISIGDQTDYADSAKG

SEQ ID NO: 152 - protein, 16 aa, synthetic construct
  REGION 1..16: note = CDR2 of TCE ISVD (Kabat)
HISIGDQTDYANSAKG

SEQ ID NO: 153 - protein, 16 aa, synthetic construct
  REGION 1..16: note = CDR2 of TCE ISVD (Kabat)
HISIGDQTDYAPSAKG

SEQ ID NO: 154 - protein, 16 aa, synthetic construct
  REGION 1..16: note = CDR2 of TCE ISVD (Kabat)
HISIGDQTDYAKSAKG

SEQ ID NO: 155 - protein, 16 aa, synthetic construct
  REGION 1..16: note = CDR2 of TCE ISVD (Kabat)
HISIGDQTDYARSAKG

SEQ ID NO: 156 - protein, 16 aa, synthetic construct
  REGION 1..16: note = CDR2 of TCE ISVD (Kabat)
HISIGDQTDYAISAKG

SEQ ID NO: 157 - protein, 16 aa, synthetic construct
  REGION 1..16: note = CDR2 of TCE ISVD (Kabat)
HISIGDQTDYATSAKG

SEQ ID NO: 158 - protein, 16 aa, synthetic construct
  REGION 1..16: note = CDR2 of TCE ISVD (Kabat)
HISIGDQTDYAHSAKG

SEQ ID NO: 159 - protein, 16 aa, synthetic construct
  REGION 1..16: note = CDR2 of TCE ISVD (Kabat)
HISIGDQTDYAVSAKG

SEQ ID NO: 160 - protein, 16 aa, synthetic construct
  REGION 1..16: note = CDR2 of TCE ISVD (Kabat)
HISIGDQTDYAESAKG

SEQ ID NO: 161 - protein, 16 aa, synthetic construct
  REGION 1..16: note = CDR2 of TCE ISVD (Kabat)
HISIGDQTDYAASAKG

SEQ ID NO: 162 - protein, 16 aa, synthetic construct
  REGION 1..16: note = CDR2 of TCE ISVD (Kabat)
HISIGDQTDYAYSAKG

SEQ ID NO: 163 - protein, 16 aa, synthetic construct
  REGION 1..16: note = CDR2 of TCE ISVD (Kabat)
HISIGDQTDYALSAKG

SEQ ID NO: 164 - protein, 16 aa, synthetic construct
  REGION 1..16: note = CDR2 of TCE ISVD (Kabat)
HISIGDQTDYAQSAKG

SEQ ID NO: 165 - protein, 16 aa, synthetic construct
  REGION 1..16: note = CDR2 of TCE ISVD (Kabat)
HISIGDQTDYAFSAKG

SEQ ID NO: 166 - protein, 16 aa, synthetic construct
  REGION 1..16: note = CDR2 of TCE ISVD (Kabat)
HISIGDQTDYASSAKG

SEQ ID NO: 167 - protein, 9 aa, synthetic construct
  REGION 1..9: note = CDR3 of TCE ISVD (Kabat)
LSRIWPYDY

SEQ ID NO: 168 - protein, 9 aa, synthetic construct
  REGION 1..9: note = CDR3 of TCE ISVD (Kabat)
LSRIYPYDY

SEQ ID NO: 169 - protein, 9 aa, synthetic construct
  REGION 1..9: note = CDR3 of TCE ISVD (Kabat)
LSRIAPYDY

SEQ ID NO: 170 - protein, 9 aa, synthetic construct
  REGION 1..9: note = CDR3 of TCE ISVD (Kabat)
LSRIPPYDY

SEQ ID NO: 171 - protein, 9 aa, synthetic construct
  REGION 1..9: note = CDR3 of TCE ISVD (Kabat)
LSRIDPYDY

SEQ ID NO: 172 - protein, 9 aa, synthetic construct
  REGION 1..9: note = CDR3 of TCE ISVD (Kabat)
LSRIQPYDY

SEQ ID NO: 173 - protein, 9 aa, synthetic construct
  REGION 1..9: note = CDR3 of TCE ISVD (Kabat)
LSRIEPYDY

SEQ ID NO: 174 - protein, 9 aa, synthetic construct
  REGION 1..9: note = CDR3 of TCE ISVD (Kabat)
LSRIRPYDY

SEQ ID NO: 175 - protein, 9 aa, synthetic construct
  REGION 1..9: note = CDR3 of TCE ISVD (Kabat)
LSRIFPYDY

SEQ ID NO: 176 - protein, 9 aa, synthetic construct
  REGION 1..9: note = CDR3 of TCE ISVD (Kabat)
LSRISPYDY

SEQ ID NO: 177 - protein, 9 aa, synthetic construct
  REGION 1..9: note = CDR3 of TCE ISVD (Kabat)
LSRIGPYDY

SEQ ID NO: 178 - protein, 9 aa, synthetic construct
  REGION 1..9: note = CDR3 of TCE ISVD (Kabat)
LSRITPYDY

SEQ ID NO: 179 - protein, 9 aa, synthetic construct
  REGION 1..9: note = CDR3 of TCE ISVD (Kabat)
LSRIHPYDY

SEQ ID NO: 180 - protein, 9 aa, synthetic construct
  REGION 1..9: note = CDR3 of TCE ISVD (Kabat)
LSRIVPYDY

SEQ ID NO: 181 - protein, 9 aa, synthetic construct
  REGION 1..9: note = CDR3 of TCE ISVD (Kabat)
LSRIKPYDY

SEQ ID NO: 182 - protein, 17 aa, synthetic construct
  REGION 1..17: note = Peptide fragment
VAHISIGDQTDYADSAK

SEQ ID NO: 183 - protein, 21 aa, synthetic construct
  REGION 1..21: note = Peptide fragment
NTVYLQMNSLRPEDTAAYYCR

SEQ ID NO: 184 - protein, 27 aa, synthetic construct
  REGION 1..27: note = Peptide fragment
IWPYDYWGQGTLVTVSSAAAHHHHHHH

SEQ ID NO: 185 - protein, 19 aa, synthetic construct
  REGION 1..19: note = Peptide fragment T14
LSCAASGFTFSSFAMTWVR

SEQ ID NO: 186 - protein, 11 aa, synthetic construct
  REGION 1..11: note = Peptide fragment T16
GLEWVATITNK

SEQ ID NO: 187 - protein, 12 aa, synthetic construct
  REGION 1..12: note = Peptide fragment T27
SVFSSSTMEWYR

SEQ ID NO: 188 - protein, 61 aa, synthetic construct
  REGION 1..61: note = Peptide fragment T36
NTVYLQMNSLRPEDTALYYCASGVAWGQGTLVTVSSGGGGSGGGSEVQLVESGGGWQPGGS

SEQ ID NO: 189 - protein, 23 aa, synthetic construct
  REGION 1..23: note = Peptide fragment T40
GPEWVSSISGSGSDTLYLADSVK

SEQ ID NO: 190 - protein, 4 aa, synthetic construct
KERE

SEQ ID NO: 191 - protein, 4 aa, synthetic construct
KQRE

SEQ ID NO: 192 - protein, 4 aa, synthetic construct
GLEW

SEQ ID NO: 193 - protein, 5 aa, synthetic construct
KEREL

SEQ ID NO: 194 - protein, 5 aa, synthetic construct
KEREF

SEQ ID NO: 195 - protein, 5 aa, synthetic construct
KQREL

SEQ ID NO: 196 - protein, 5 aa, synthetic construct
KQREF

SEQ ID NO: 197 - protein, 5 aa, synthetic construct
KEREG

SEQ ID NO: 198 - protein, 5 aa, synthetic construct
KQREW

SEQ ID NO: 199 - protein, 5 aa, synthetic construct
KQREG

SEQ ID NO: 200 - protein, 4 aa, synthetic construct
TERE

SEQ ID NO: 201 - protein, 5 aa, synthetic construct
TEREL

SEQ ID NO: 202 - protein, 4 aa, synthetic construct
TQRE

SEQ ID NO: 203 - protein, 5 aa, synthetic construct
TQREL

SEQ ID NO: 204 - protein, 4 aa, synthetic construct
KECE

SEQ ID NO: 205 - protein, 5 aa, synthetic construct
KECEL

SEQ ID NO: 206 - protein, 5 aa, synthetic construct
KECER

SEQ ID NO: 207 - protein, 4 aa, synthetic construct
KQCE

SEQ ID NO: 208 - protein, 5 aa, synthetic construct
KQCEL

SEQ ID NO: 209 - protein, 4 aa, synthetic construct
RERE

SEQ ID NO: 210 - protein, 5 aa, synthetic construct
REREG

SEQ ID NO: 211 - protein, 4 aa, synthetic construct
RQRE

SEQ ID NO: 212 - protein, 5 aa, synthetic construct
RQREL

SEQ ID NO: 213 - protein, 5 aa, synthetic construct
RQREF

SEQ ID NO: 214 - protein, 5 aa, synthetic construct
RQREW

SEQ ID NO: 215 - protein, 4 aa, synthetic construct
QERE

SEQ ID NO: 216 - protein, 5 aa, synthetic construct
QEREG

SEQ ID NO: 217 - protein, 4 aa, synthetic construct
QQRE

SEQ ID NO: 218 - protein, 5 aa, synthetic construct
QQREW

SEQ ID NO: 219 - protein, 5 aa, synthetic construct
QQREL

SEQ ID NO: 220 - protein, 5 aa, synthetic construct
QQREF

SEQ ID NO: 221 - protein, 4 aa, synthetic construct
KGRE

SEQ ID NO: 222 - protein, 5 aa, synthetic construct
KGREG

SEQ ID NO: 223 - protein, 4 aa, synthetic construct
KDRE

SEQ ID NO: 224 - protein, 5 aa, synthetic construct
KDREV

SEQ ID NO: 225 - protein, 5 aa, synthetic construct
DECKL

SEQ ID NO: 226 - protein, 5 aa, synthetic construct
NVCEL

SEQ ID NO: 227 - protein, 4 aa, synthetic construct
GVEW

SEQ ID NO: 228 - protein, 4 aa, synthetic construct
EPEW

SEQ ID NO: 229 - protein, 4 aa, synthetic construct
GLER

SEQ ID NO: 230 - protein, 4 aa, synthetic construct
DQEW

SEQ ID NO: 231 - protein, 4 aa, synthetic construct
DLEW

SEQ ID NO: 232 - protein, 4 aa, synthetic construct
GIEW

SEQ ID NO: 233 - protein, 4 aa, synthetic construct
ELEW

SEQ ID NO: 234 - protein, 4 aa, synthetic construct
GPEW

SEQ ID NO: 235 - protein, 4 aa, synthetic construct
EWLP

SEQ ID NO: 236 - protein, 4 aa, synthetic construct
GPER

SEQ ID NO: 237 - protein, 4 aa, synthetic construct
GLER